(19) United States
(12) Patent Application Publication
(10) Pub. No.: US 2022/0089612 A1
(43) Pub. Date: Mar. 24, 2022

(54) HETEROCYCLIC COMPOUNDS AS PRMT5 INHIBITORS

(71) Applicant: ANGEX PHARMACEUTICAL, INC., North Brunswick, NJ (US)

(72) Inventors: Wen-Lian Wu, Green Brook, NJ (US); Zhiqiang Yang, Westfield, NJ (US); Francis Lee, Yardley, PA (US); John Qiang Tan, North Brunswick, NJ (US)

(21) Appl. No.: 17/532,964

(22) Filed: Nov. 22, 2021

Related U.S. Application Data
(63) Continuation of application No. PCT/US2020/034711, filed on May 27, 2020.
Application No. PCT/US2020/034711 is a continuation-in-part of application No. PCT/US2018/058721, filed on Nov. 1, 2018.
(60) Provisional application No. 62/854,435, filed on May 30, 2019.
(60) Provisional application No. 62/966,337, filed on Jan. 27, 2020.
(60) Provisional application No. 62/594,898, filed on Dec. 5, 2017.

(51) Int. Cl. C07D 519/00; C07D 487/04; C07H 19/23

(52) U.S. Cl. CPC C07D 519/00; C07H 19/23; C07D 487/04

(57) ABSTRACT

The present disclosure describes novel heterocyclic PRMT5 inhibitors and methods for preparing them. The pharmaceutical compositions comprising such PRMT5 inhibitors and methods of using them for treating cancer, infectious diseases, and other PRMT5 associated disorders are also described.

### CROSS-REFERENCE TO RELATED APPLICATIONS

This application is a continuation of the International Patent Application No. PCT/US2020/034711, filed May 27, 2020; which claims the benefit of U.S. Provisional Application Nos. 62/854,435, filed May 30, 2019, and 62/966,337, filed Jan. 27, 2020; International Patent Application No. PCT/US2020/034711 is also a continuation-in-part of the International Patent Application No. PCT/US2018/058721, filed Nov. 1, 2018, which claims the benefit of U.S. Provisional Application No. 62/594,898, filed Dec. 5, 2017; all of which are incorporated by reference by their entirety.

### FIELD

The present disclosure relates to heterocyclic compounds, such as (1R,2S,3R,5S)-3-(4-amino-7H-pyrrolo[2,3-d]pyrimidin-7-yl)-5-(2-(2,3-dihydro-1H-pyrrolo[2,3-b]quinolin-7-yl)ethyl)-cyclopentane-1,2-diol (1-7), as PRMT5 inhibitors, and pharmaceutical compositions comprising such compounds. The present disclosure also relates to the use of the compounds and compositions to treat cancer, infectious diseases, and other disorders.

### BACKGROUND

Protein arginine N-methyltransferase 5 (PRMT5), the human homolog of Skb1 (Schizosaccharomyces pombe) and Hsl7 (Saccharomyces cerevisiae), was discovered in a yeast two-hybrid screen as a Janus kinase 2 (JAK2) binding protein. PRMT5 catalyzes the transfer of methyl group from the essential co-factor S-adenosylmethionine to methylate the arginine N-guanidine group of various proteins. Substrate proteins for PRMT5 include histones, transcriptional elongation factors, kinases, and tumor suppressors, for example, histone H4, histone H3, and non-histone proteins such as FGF-216, NF-kB17, HOXA918, and p53. PRMT5 is involved in the transcriptional repression of a number of tumor suppressor genes including suppressor of tumorigenicity 7 (ST7), nonmetastatic 23 (NM23), retinoblastoma (Rb) family, and programmed cell death 4 (PDCD4).

PRMT5 has recently emerged as a promising drug target due to its frequent overexpression in a variety of malignancies including glioma, lung cancer, melanoma, mantle cell lymphoma, multiple endocrine neoplasia, prostate and gastric cancer, as well as its synthetic lethal relationship with methylthioadenosine phosphorylase (MTAP). Importantly, in addition to overexpression, PRMT5 localization differs between normal and tumor tissues and between tumor subtypes. This is indicative that its compartment-specific functions likely regulate distinct molecular programs and are therefore associated with diverse phenotypic outcomes. Thus, the identification and development of small-molecules that inhibit PRMT5 activity will serve as therapeutic approach for the treatment of a variety of PRMT5-related diseases or disorders, such as cancer.

### SUMMARY

This disclosure relates to heterocyclic compounds comprising at least three ring systems, such as a compound of Formula 1, certain optionally substituted (1S,2R,3S,5R)-3-(2-(2,3-dihydro-1H-pyrrolo[2,3-b]quinolin-7-yl)ethyl)-5-(7H-pyrrolo[2,3-d]pyrimidin-7-yl)cyclopentane-1,2-diol, optionally substituted (1S,2R,3S,5R)-3-(2-(2,3-dihydroimidazo[1,2-c]quinazolin-8-yl)ethyl)-5-(7H-pyrrolo[2,3-d]pyrimidin-7-yl)cyclopentane-1,2-diol, optionally substituted (1S,2R,3S,5R)-3-(2-(imidazo[1,2-c]quinazolin-8-yl)ethyl)-5-(7H-pyrrolo[2,3-d]pyrimidin-7-yl)cyclopentane-1,2-diol, optionally substituted (1S,2R,3S,5R)-3-(2-((1aS,7bR)-1a,7b-dihydro-1H-cyclopropa[c]quinolin-5-yl)ethyl)-5-(7H-pyrrolo[2,3-d]pyrimidin-7-yl)cyclopentane-1,2-diol, optionally substituted (1S,2R,3S,5R)-3-(2-((1aR,7bS)-1a,7b-dihydro-1H-cyclopropa[c]quinolin-5-yl)ethyl)-5-(7H-pyrrolo[2,3-d]pyrimidin-7-yl)cyclopentane-1,2-diol, optionally substituted (1S,2R,3S,5R)-3-(2-(1H-pyrazolo[3,4-b]quinolin-7-yl)ethyl)-5-(7H-pyrrolo[2,3-d]pyrimidin-7-yl)cyclopentane-1,2-diol, optionally substituted (2R,3S,4R,5R)-2-(2-(2,3-dihydro-1H-pyrrolo[2,3-b]quinolin-7-yl)ethyl)-5-(7H-pyrrolo[2,3-d]pyrimidin-7-yl)tetrahydrofuran-3,4-diol, optionally substituted (2R,3S,4R,5R)-2-(2-(2,3-dihydroimidazo[1,2-c]quinazolin-8-yl)ethyl)-5-(7H-pyrrolo[2,3-d]pyrimidin-7-yl)tetrahydrofuran-3,4-diol, optionally substituted (2R,3S,4R,5R)-2-(2-(imidazo[1,2-c]quinazolin-8-yl)ethyl)-5-(7H-pyrrolo[2,3-d]pyrimidin-7-yl)tetrahydrofuran-3,4-diol, optionally substituted (1S,2R,3S,5R)-3-(2-(1H-pyrrolo[2,3-b]quinolin-7-yl)ethyl)-5-(7H-pyrrolo[2,3-d]pyrimidin-7-yl)cyclopentane-1,2-diol, optionally substituted (1S,2R,3S,5R)-3-(2-(3,4-dihydro-1H-[1,2]oxazino[3,4-b]quinolin-8-yl)ethyl)-5-(7H-pyrrolo[2,3-d]pyrimidin-7-yl)cyclopentane-1,2-diol, optionally substituted (1S,2R,3S,5R)-3-(2-(1,3-dihydroisoxazolo[3,4-b]quinolin-7-yl)ethyl)-5-(7H-pyrrolo[2,3-d]pyrimidin-7-yl)cyclopentane-1,2-diol, optionally substituted (1R,2S,3R,5S)-3-(7H-pyrrolo[2,3-d]pyrimidin-7-yl)-5-(2-(1,3,4,5-tetrahydro-[1,2]oxazepino[3,4-b]quinolin-9-yl)ethyl)cyclopentane-1,2-diol, optionally substituted (1S,2R,3S,5R)-3-(2-(3H-imidazo[4,5-b]quinolin-6-yl)ethyl)-5-(7H-pyrrolo[2,3-d]pyrimidin-7-yl)cyclopentane-1,2-diol, optionally substituted (1S,2R,3S,5R)-3-(2-(3H-[1,2,3]triazolo[4,5-b]quinolin-6-yl)ethyl)-5-(7H-pyrrolo[2,3-d]pyrimidin-7-yl)cyclopentane-1,2-diol, optionally substituted (1S,2R,3S,5R)-3-(2-(2,3-dihydro-1H-pyrazolo[3,4-b]quinolin-7-yl)ethyl)-5-(7H-pyrrolo[2,3-d]pyrimidin-7-yl)cyclopentane-1,2-diol, optionally substituted (1R,2S,3R,5S)-3-(7H-pyrrolo[2,3-d]pyrimidin-7-yl)-5-(2-(1,2,3,4-tetrahydrobenzo[b][1,8]naphthyridin-8-yl)ethyl)cyclopentane-1,2-diol, optionally substituted (1R,2S,3R,5S)-3-(7H-pyrrolo[2,3-d]pyrimidin-7-yl)-5-(2-(2,3,4,5-tetrahydro-1H-azepino[2,3-b]quinolin-9-yl)ethyl)cyclopentane-1,2-diol, optionally substituted (2R,3S,4R,5R)-2-(2-(1H-pyrazolo[3,4-b]quinolin-7-yl)ethyl)-5-(7H-pyrrolo[2,3-d]pyrimidin-7-yl)tetrahydrofuran-3,4-diol, optionally substituted (2R,3R,4S,5R)-2-(7H-pyrrolo[2,3-d]pyrimidin-7-yl)-5-(2-(1,2,3,4-tetrahydrobenzo[b][1,8]naphthyridin-8-yl)ethyl)tetrahydrofuran-3,4-diol, or optionally substituted (1R,2S,3R,5S)-3-(7H-pyrrolo[2,3-d]pyrimidin-7-yl)-5-(2-(1,2,3,4-tetrahydropyridazino[3,4-b]quinolin-8-yl)ethyl)cyclopentane-1,2-diol, or any one of other novel compounds described herein, or a pharmaceutically acceptable salt thereof, or a combination thereof (referred to collectively herein as a “subject compound”).

Some embodiments include a compound represented by Formula 1:

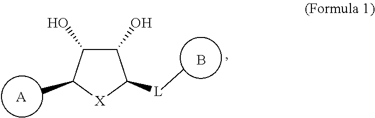

or a pharmaceutically acceptable salt thereof; wherein

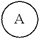

(Ring A) is an optionally substituted 4-amino-7H-pyrrolo[2,3-d]pyrimidin-7-yl;

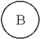

(Ring B) is an optionally substituted fused tricyclic heterocyclic ring system containing 1, 2, 3, 4, or 5 ring N atoms, 0 or 1 ring O atom, and a fused benzene ring, wherein the fused benzene ring is directly attached to L; X is —O—, —CH₂—, or —CF₂—; L is optionally substituted C₁₋₃ hydrocarbylene, optionally substituted —O—C₁₋₂ hydrocarbylene-, optionally substituted —S—C₁₋₂ hydrocarbylene-, or optionally substituted —NR^{A}—C₁₋₂ hydrocarbylene; and R^{A} is H, C₁₋₆ hydrocarbyl, C₁₋₆ heteroaryl, C₁₋₆ heterocycloalkyl, —C(O)—C₁₋₆ alkyl, —C(O)NH—C₁₋₆ alkyl, or —C(O)OC₁₋₆ alkyl.

Some embodiments include use of a compound described herein, such as a subject compound in the manufacture of a medicament for the treatment of cancer, infectious diseases, and other PRMT5 related disorders.

Some embodiments include a pharmaceutical composition comprising a therapeutically effective amount of a subject compound in combination with at least one pharmaceutically acceptable carrier.

Some embodiments include a process for making a pharmaceutical composition comprising combining a subject compound and at least one pharmaceutically acceptable carrier.

Some embodiments include a method of treating cancer, infectious diseases, and other PRMT5 related disorders comprising administering a subject compound to a patient in need thereof.

Some embodiments include use of a subject compound in the manufacture of a medicament for the treatment of cancer, infectious diseases, and other PRMT5 related disorders.

Some embodiments include a kit containing a subject compound and a label with instructions to use the subject compound, or a composition or dosage form containing the subject compound, for the treatment of cancer, infectious diseases, and other PRMT5 related disorders.

### DETAILED DESCRIPTION

Unless otherwise indicated, any reference to a compound herein by structure, name, or any other means, includes pharmaceutically acceptable salts, such as sodium, potassium, and ammonium salts; prodrugs, such as ester prodrugs; alternate solid forms, such as polymorphs, solvates, hydrates, etc.; tautomers; or any other chemical species that may rapidly convert to a compound described herein under conditions in which the compounds are used as described herein.

If stereochemistry is not indicated, a name or structural depiction includes any stereoisomer or any mixture of stereoisomers.

In some embodiments, a compound of Formula 1 is a single enantiomer.

In some embodiments, a subject compound described herein contains one or more deuterium.

Unless otherwise indicated, when a compound or chemical structural feature such as aryl is referred to as being “optionally substituted”, it includes a feature that has no substituents (i.e. unsubstituted), or a feature that is “substituted”, meaning that the feature has one or more substituents. The term “substituent” is broad, and includes a moiety that occupies a position normally occupied by one or more hydrogen atoms attached to a parent compound or structural feature. In some embodiments, a substituent may be an ordinary organic moiety known in the art, which may have a molecular weight (e.g. the sum of the atomic masses of the atoms of the substituent) of 15 g/mol to 50 g/mol, 15 g/mol to 60 g/mol, 15 g/mol to 70 g/mol, 15 g/mol to 80 g/mol, 15 g/mol to 90 g/mol, 50 g/mol to 60 g/mol, 60 g/mol to 70 g/mol, 70 g/mol to 80 g/mol, 80 g/mol to 90 g/mol, 90 g/mol to 100 g/mol, 15 g/mol to 100 g/mol, 15 g/mol to 150 g/mol, 15 g/mol to 200 g/mol, 15 g/mol to 300 g/mol, or 15 g/mol to 500 g/mol. In some embodiments, a substituent comprises, or consists of: 0-30, 0-20, 0-10, or 0-5 carbon atoms; and 0-30, 0-20, 0-10, or 0-5 heteroatoms, wherein each heteroatom may independently be: N, O, S, P, Si, F, Cl, Br, or I; provided that the substituent includes one C, N, O, S, P, Si, F, Cl, Br, or I atom and N, S and P can be optionally oxidized. Examples of substituents include, but are not limited to, deuterium, tritium, alkyl, alkenyl, alkynyl, heteroalkyl, heteroalkenyl, heteroalkynyl, aryl, heteroaryl, hydroxy, alkoxy, aryloxy, acyl, acyloxy, alkylcarboxylate, thiol, alkylthio, cyano, halo, thiocarbonyl, O-carbamyl, N-carbamyl, O-thiocarbamyl, N-thiocarbamyl, C-amido, N-amido, S-sulfonamido, N-sulfonamido, isocyanato, thiocyanato, isothiocyanato, nitro, silyl, sulfenyl, sulfinyl, sulfonyl, haloalkyl, haloalkoxyl, trihalomethanesulfonyl, trihalomethanesulfonamido, amino, phosphonic acid, etc.

For convenience, the term “molecular weight” is used with respect to a moiety or part of a molecule to indicate the sum of the atomic masses of the atoms in the moiety or part of a molecule, even though it may not be a complete molecule.

The structures associated with some of the chemical names referred to herein, such as Ring A and Ring B, are depicted below. These structures may be unsubstituted, as shown below, or substituted with a substituent that may independently be in any position normally occupied by a hydrogen atom when the structure is unsubstituted. Unless a point of attachment is indicated by

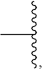

attachment may occur at any position normally occupied by a hydrogen atom.

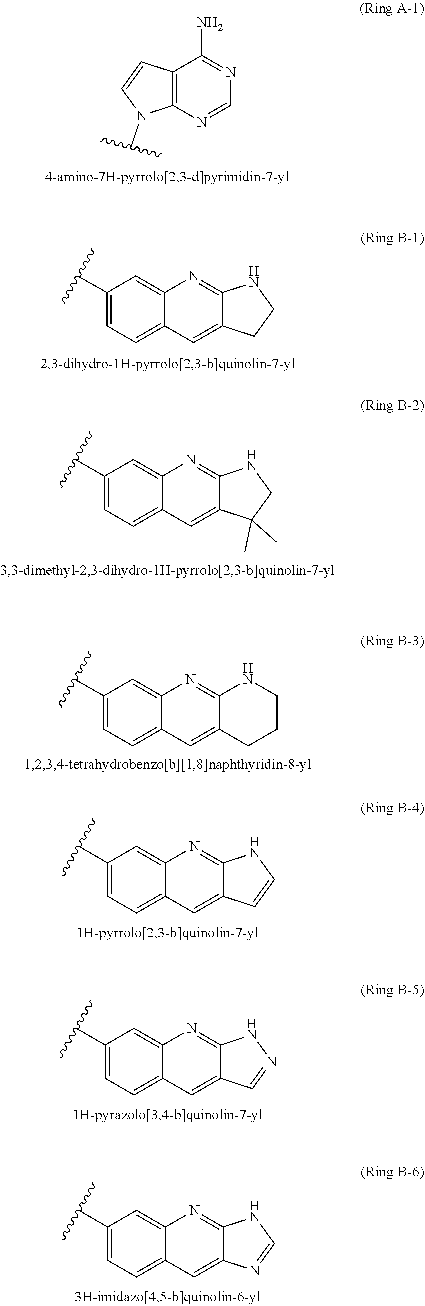

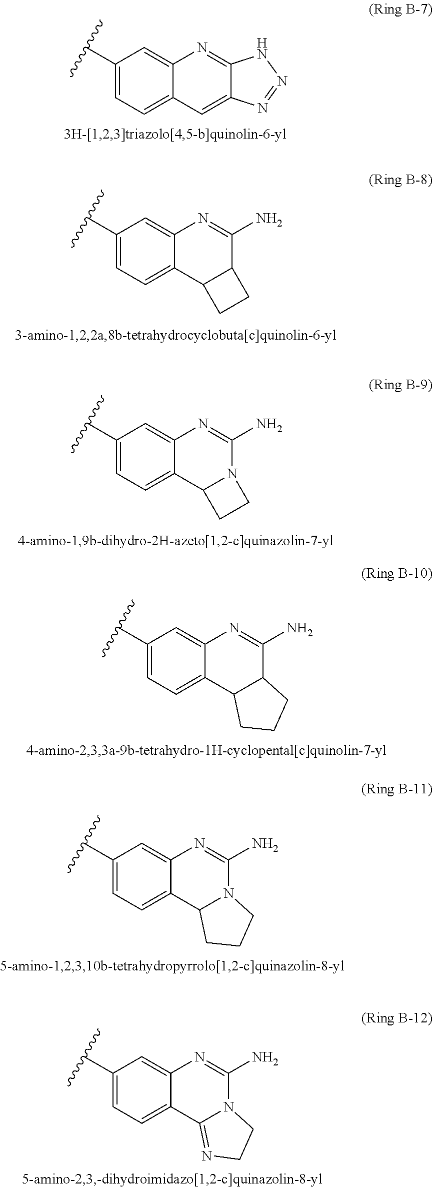 [SMILES: *c1ccc2cc3nn[nH]c3nc2c1 ; *c1ccc2c(c1)N=C(N)C1CCC21 ; *c1ccc2c(c1)N=C(N)N1CCC21 ; *c1ccc2c(c1)N=C(N)C1CCCC21 ; *c1ccc2c(c1)N=C(N)N1CCCC21 ; *c1ccc2c(c1)N=C(N)N1CCN=C21]

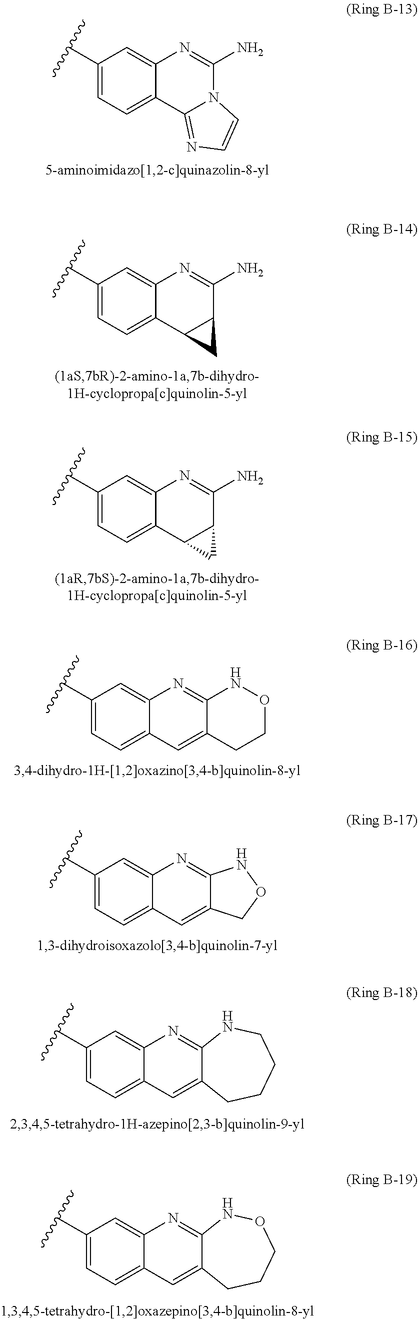

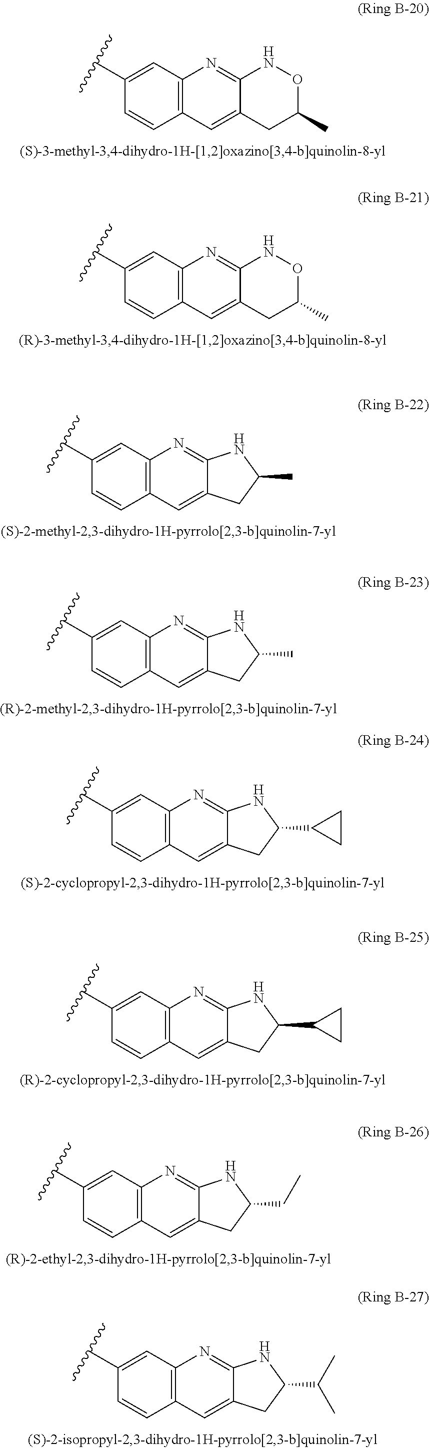

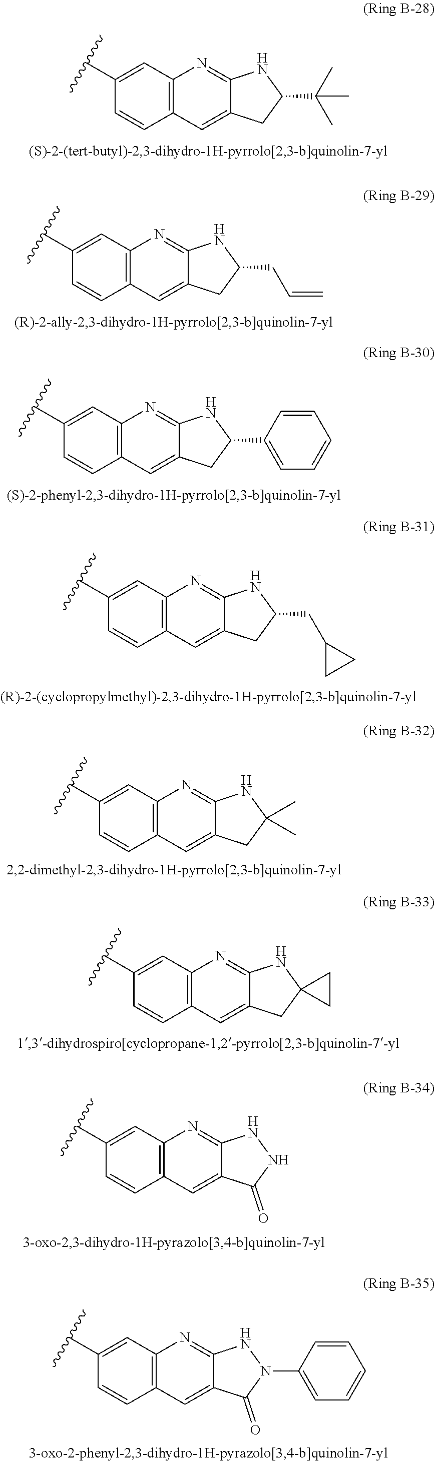

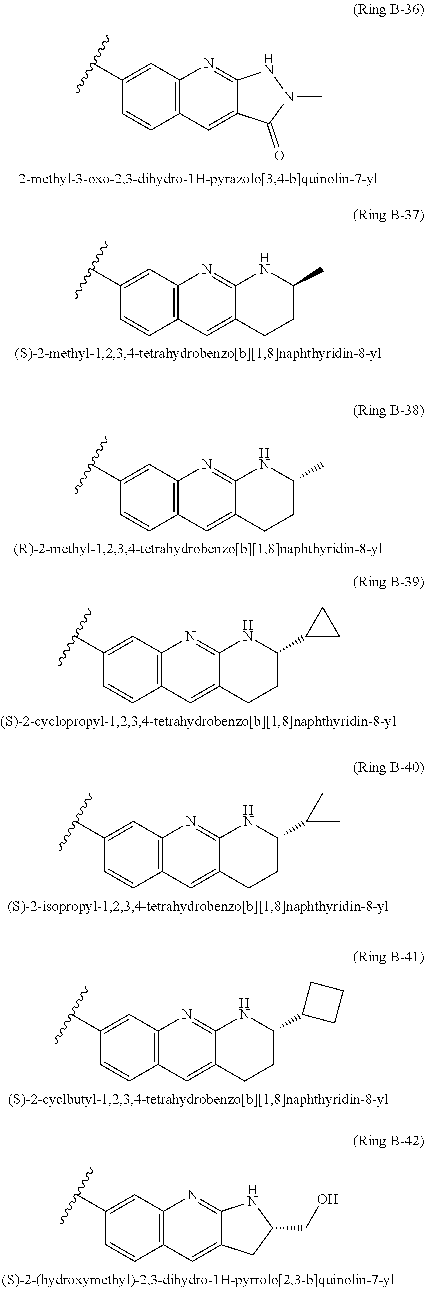

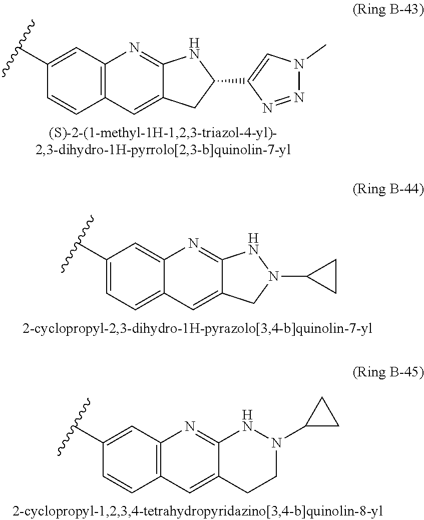 [SMILES: *c1ccc2cc3c(nc2c1)N[C@H](c1cn(C)nn1)C3 ; *c1ccc2cc3c(nc2c1)NN(C1CC1)C3 ; *c1ccc2cc3c(nc2c1)NN(C1CC1)CC3]

In some embodiments, Ring A of Formula 1 comprises:

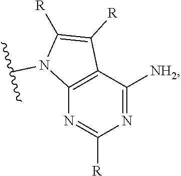

wherein each R is independently H, F, Cl, Br, I, —NR^{A}R^{B}, C₁₋₆ hydrocarbyl, —OH, —CN, or —O—C₁₋₆ alkyl; and wherein each R^{A} and each R^{B} are independently H, C₁₋₆ hydrocarbyl, C₁₋₆ heteroaryl, C₁₋₆ heterocycloalkyl, —C(O)—C₁₋₆ alkyl, —C(O)NH—C₁₋₆ alkyl, or —C(O)OC₁₋₆ alkyl.

With respect to any relevant structural representation, such as Formula 1, Ring A is an optionally substituted 4-amino-7H-pyrrolo[2,3-d]pyrimidin-7-yl. In some embodiments, any or each of the substituents of Ring A may have a molecular weight of at least 15 g/mol, and up to: 50 g/mol, 60 g/mol, 70 g/mol, 80 g/mol, 90 g/mol, 100 g/mol, or 300 g/mol. Potential substituents of Ring A may include —OH; —CN; halo, such as F, Cl, Br, I; hydrocarbyl, such as methyl, C₂ alkyl, C₂ alkenyl, C₂ alkynyl, C₃ alkyl, C₃ cycloalkyl, C₃ alkenyl, C₃ alkynyl, C₄ alkyl, C₄ cycloalkyl, C₄ alkenyl, C₄ alkynyl, C₅ alkyl, C₅ cycloalkyl, C₅ alkenyl, C₅ alkynyl, C₆ alkyl, C₆ cycloalkyl, C₆ alkenyl, C₆ alkynyl, phenyl, etc.; CN₀₋₁O₀₋₂F₀₋₃H₀₋₄; C₂N₀₋₁O₀₋₃F₀₋₅H₀₋₆; C₃N₀₋₁O₀₋₃F₀₋₇H₀₋₈; C₄N₀₋₁O₀₋₃F₀₋₉H₀₋₁₀; C₅N₀₋₁O₀₋₃F₀₋₁₁ H₀₋₁₂; C₆N₀₋₁O₀₋₃F₀₋₁₃H₀₋₁₄; etc. In some embodiments, Ring A is optionally substituted 4-amino-7H-pyrrolo[2,3-d]pyrimidin-7-yl having 1, 2, or 3 substituents, such as 4-amino-7H-pyrrolo[2,3-d]pyrimidin-7-yl substituted with F, Cl, Br, C₁₋₆ alkyl, —CO₂H, —CN, —CO—C₁₋₆-alkyl, —C(O)O—C₁₋₆-alkyl, C₁₋₆ alkyl-OH, OH, NH₂, etc. In some embodiments, Ring A is unsubstituted 4-amino-7H-pyrrolo[2,3-d]pyrimidin-7-yl.

With respect to Formula 1, in some embodiments, Ring A is represented by Formula A1:

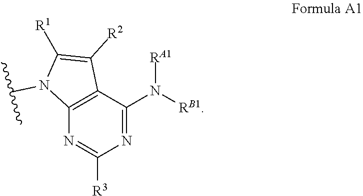

With respect to any relevant structural representation, such as Formula A1, R¹ is H or any substituent, such as R^{A}, F, Cl, —CN, —OR^{A}, CF₃, —NO₂, —NR^{A}R^{B}, —COR^{A}, CO₂R^{A}, —OCOR^{A}, —NR^{A}COR^{B}, or —CONR^{A}R^{B}, etc., wherein R^{A} and R^{B} are each H, C₁₋₆hydrocarbyl, C₁₋₆ heteroaryl, C₁₋₆ heterocycloalkyl, —C(O)—C₁₋₆ alkyl, —C(O)NH—C₁₋₆ alkyl, or —C(O)OC₁₋₆ alkyl. Some of the structures with attachment points are shown below. In some embodiments, R¹ may be H; F; Cl; —CN; CF₃; OH; NH₂; C₁₋₆ alkyl, such as methyl, ethyl, any one of the propyl isomers (e.g. n-propyl and isopropyl), cyclopropyl, any one of the butyl isomers, any one of the cyclobutyl isomers (e.g. cyclobutyl and methylcyclopropyl), any one of the pentyl isomers, any one of the cyclopentyl isomers, any one of the hexyl isomers, and any one of the cyclohexyl isomers, etc.; or C₁₋₆ alkoxy, such as —O-methyl, —O-ethyl, any one of the isomers of —O-propyl, —O-cyclopropyl, any one of the isomers of —O-butyl, any one of the isomers of —O-cyclobutyl, any one of the isomers of —O-pentyl, any one of the isomers of —O-cyclopentyl, any one of the isomers of —O-hexyl, any one of the isomers of —O-cyclohexyl, etc. In some embodiments, R¹ may be H, F, C, or NH₂. In some embodiments, R¹ may be H.

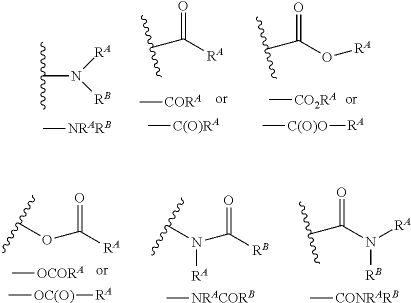

With respect to any relevant structural representation, each R^{A1} may independently be H, or C₁₋₁₂ hydrocarbyl, such as C₁₋₁₂ alkyl, C₁₋₁₂ alkenyl, C₁₋₁₂ alkynyl, phenyl, etc., including: linear or branched alkyl having a formula CₐH₂ₐ₊₁, or cycloalkyl having a formula CₐH₂ₐ₋₁, wherein a is 1, 2, 3, 4, 5, 6, 7, 8, 9, 10, 11, or 12, such as linear or branched alkyl with a formula: CH₃, C₂H₅, C₃H₇, C₄H₉, C₅H₁₁, C₆H₁₃, C₇H₁₅, C₈H₁₇, C₉H₁₉, C₁₀H₂₁, etc., or cycloalkyl with a formula: C₃H₅, C₄H₇, C₅H₉, C₆H₁₁, C₇H₁₃, CH₁₅, C₉H₁₇, C₁₀H₁₉, etc. In some embodiments, R^{A1} may be H or C₁₋₆ alkyl. In some embodiments, R^{A1} may be H or C₁₋₃ alkyl. In some embodiments, R^{A1} may be H or CH₃. In some embodiments, R^{A1} may be H.

With respect to any relevant structural representation, each R^{B1} may independently be H, or C₁₋₁₂ hydrocarbyl, such as C₁₋₁₂ alkyl, C₁₋₁₂ alkenyl, C₁₋₁₂ alkynyl, phenyl, etc., including: linear or branched alkyl having a formula CₐH₂ₐ₊₁, or cycloalkyl having a formula CₐH₂ₐ₋₁, wherein a is 1, 2, 3, 4, 5, 6, 7, 8, 9, 10, 11, or 12, such as linear or branched alkyl with a formula: CH₃, C₂H₅, C₃H₇, C₄H₉, C₅H₁₁, C₆H₁₃, C₇H₁₅, C₈H₁₇, C₉H₁₉, C₁₀H₂₁, etc., or cycloalkyl with a formula: C₃H₅, C₄H₇, C₅H₉, C₆H₁₁, C₇H₁₃, CH₁₅, C₉H₁₇, C₁₀H₁₉, etc. In some embodiments, R^{B1} may be H or C₁₋₃ alkyl. In some embodiments, R^{B1} may be H or CH₃. In some embodiments, R^{B1} may be H.

With respect to any relevant structural representation, such as Formula A1, R² is H or any substituent, such as R^{A}, F, Cl, —CN, —OR^{A}, CF₃, —NO₂, —NR^{A}R^{B}, —COR^{A}, —CO₂R^{A}, —OCOR^{A}, —NR^{A}COR^{B}, or —CONR^{A}R^{B}, etc. In some embodiments, R² may be H, F, C, CN, CF₃, OH, NH₂, C₁₋₆ alkyl, or C₁₋₆ alkoxy. In some embodiments, R² may be H, F, Cl, or NH₂. In some embodiments, R² may be H.

With respect to any relevant structural representation, such as Formula A1, R³ is H or any substituent, such as R^{A}, F, Cl, —CN, —OR^{A}, CF₃, —NO₂, —NR^{A}R^{B}, —COR^{A}, —CO₂R^{A}, —OCOR^{A}, —NR^{A}COR^{B}, or —CONR^{A}R^{B}, etc. In some embodiments, R³ may be H, F, Cl, —CN, CF₃, OH, NH₂, C₁₋₆ alkyl, or C₁₋₆ alkoxy. In some embodiments, R³ may be H, F, Cl, or NH₂. In some embodiments, R³ may be H.

With respect to any relevant structural representation, such as Formula A1, in some embodiments, both R^{A} and R^{B} are H. In some embodiments, R¹, R², and R³ are all H. In some embodiments, R^{A}, R^{B}, R¹, R², and R³ are all H.

With respect to any relevant structural representation, such as Formula 1, Ring B is an optionally substituted fused tricyclic heterocyclic ring system containing 1, 2, 3, 4, or 5 ring N atoms, 0 or 1 ring 0 atom, and a fused benzene ring, wherein the fused benzene ring is directly attached to L. In some embodiments, any or each of the substituents of Ring B may have a molecular weight of 15 g/mol to 50 g/mol, 50 g/mol to 100 g/mol, 50 g/mol to 75 g/mol, 75 g/mol to 100 g/mol, or 100 g/mol to 300 g/mol. Potential substituents of Ring B may include halo, such as F, Cl, Br, or I; hydrocarbyl, such as methyl, C₂ alkyl, C₂ alkenyl, C₂ alkynyl, C₃ alkyl, C₃ cycloalkyl, C₃ alkenyl, C₃ alkynyl, C₄ alkyl, C₄ cycloalkyl, C₄ alkenyl, C₄ alkynyl, C₅ alkyl, C₅ cycloalkyl, C₅ alkenyl, C₅ alkynyl, C₆ alkyl, C₆ cycloalkyl, C₆ alkenyl, C₆ alkynyl, or phenyl, etc.; CN₀₋₁O₀₋₂F₀₋₃H₀₋₄; C₂N₀₋₁O₀₋₃F₀₋₅H₀₋₆; C₃N₀₋₁O₀₋₃F₀₋₇H₀₋₈; C₄N₀₋₁O₀₋₃F₀₋₉H₀₋₁₀; C₅N₀₋₁O₀₋₃F₀₋₁₁H₀₋₁₂; or C₆N₀₋₁O₀₋₃F₀₋₁₃H₀₋₁₄; etc. In some embodiments, Ring B is optionally substituted 2,3-dihydro-1H-pyrrolo[2,3-b]quinolin-7-yl, optionally substituted 1,2,3,4-tetrahydrobenzo[b][1,8]naphthyridin-8-yl, optionally substituted 1H-pyrrolo[2,3-b]quinolin-7-yl, optionally substituted 1H-pyrazolo[3,4-b]quinolin-7-yl, optionally substituted 3H-imidazo[4,5-b]quinolin-6-yl, optionally substituted 3H-[1,2,3]triazolo[4,5-b]quinolin-6-yl, optionally substituted 2,3-dihydroimidazo[1,2-c]quinazolin-8-yl, optionally substituted imidazo[1,2-c]quinazolin-8-yl, optionally substituted (1aS,7bR)-1a,7b-dihydro-1H-cyclopropa[c]quinolin-5-yl, optionally substituted (1aR,7bS)-1a,7b-dihydro-1H-cyclopropa[c]quinolin-5-yl, optionally substituted 3,4-dihydro-1H-[1,2]oxazino[3,4-b]quinolin-8-yl, optionally substituted 1,3-dihydroisoxazolo[3,4-b]quinolin-7-yl, optionally substituted 2,3,4,5-tetrahydro-1H-azepino[2,3-b]quinolin-9-yl, optionally substituted 1,3,4,5-tetrahydro-[1,2]oxazepino[3,4-b]quinolin-9-yl, optionally substituted 3-oxo-2,3-dihydro-1H-pyrazolo[3,4-b]quinolin-7-yl, or optionally substituted 1,2,3,4-tetrahydropyridazino[3,4-b]quinolin-8-yl. In some embodiments, Ring B is optionally substituted 2,3-dihydro-1H-pyrrolo[2,3-b]quinolin-7-yl having 0, 1, 2, or 3, 4, 5, 6, 7, 8, or 9 substituents, such as 2,3-dihydro-1H-pyrrolo[2,3-b]quinolin-7-yl substituted with F, Cl, Br, C₁₋₆ alkyl, —CO₂H, —CN, —CO—C₁₋₆-alkyl, —C(O)O—C₁₋₆-alkyl, —C₁₋₆ alkyl-OH, OH, NH₂, etc. In some embodiments, Ring B is 2,3-dihydro-1H-pyrrolo[2,3-b]quinolin-7-yl having 2 substituents. In some embodiments, Ring B is 2,3-dihydro-1H-pyrrolo[2,3-b]quinolin-7-yl having 1 substituent. In some embodiments, Ring B is unsubstituted 2,3-dihydro-1H-pyrrolo[2,3-b]quinolin-7-yl. In some embodiments, Ring B is 2,3-dihydro-1H-pyrrolo[2,3-b]quinolin-7-yl having 2 substituents that are both methyl groups. In some embodiments, Ring B is 2,3-dihydro-1H-pyrrolo[2,3-b]quinolin-7-yl having 2 substituents at a same ring carbon atom that are two methylene groups linked together and together with the ring carbon atom to which they are attached to form a spiro cyclopropyl. In some embodiments, Ring B is 2,3-dihydro-1H-pyrrolo[2,3-b]quinolin-7-yl having 1 substituent that is methyl, ethyl, isopropyl, tert-butyl, —CH₂CH═CH2, or cyclopropyl. In some embodiments, Ring B is 2,3-dihydro-1H-pyrrolo[2,3-b]quinolin-7-yl having 1 substituent that is methyl. In some embodiments, Ring B is optionally substituted 1,2,3,4-tetrahydrobenzo[b][1,8]naphthyridin-8-yl having 0, 1, 2, or 3, 4, 5, 6, 7, 8, 9, 10, or 11 substituents, such as 1,2,3,4-tetrahydrobenzo[b][1,8]naphthyridin-8-yl substituted with F, Cl, Br, C₁₋₆ alkyl, —CO₂H, —CN, —CO—C₁₋₆-alkyl, —C(O)O—C₁₋₆-alkyl, —C₁₋₆ alkyl-OH, OH, NH₂, etc. In some embodiments, Ring B is 1,2,3,4-tetrahydrobenzo[b][1,8]naphthyridin-8-yl having 2 substituents. In some embodiments, Ring B is 1,2,3,4-tetrahydrobenzo[b][1,8]naphthyridin-8-yl having 1 substituent. In some embodiments, Ring B is unsubstituted 1,2,3,4-tetrahydrobenzo[b][1,8]naphthyridin-8-yl. In some embodiments, Ring B is 1,2,3,4-tetrahydrobenzo[b][1,8]naphthyridin-8-yl having 1 substituent that is methyl or cyclopropyl. In some embodiments, Ring B is 1,2,3,4-tetrahydrobenzo[b][1,8]naphthyridin-8-yl having 1 substituent that is methyl. In some embodiments, Ring B is 1,2,3,4-tetrahydrobenzo[b][1,8]naphthyridin-8-yl having 1 substituent that is cyclopropyl. In some embodiments, Ring B is 3,4-dihydro-1H-[1,2]oxazino[3,4-b]quinolin-8-yl. In some embodiments, Ring B is 1,3-dihydroisoxazolo[3,4-b]quinolin-7-yl. In some embodiments, Ring B is 3H-imidazo[4,5-b]quinolin-6-yl. In some embodiments, Ring B is 2,3,4,5-tetrahydro-1H-azepino[2,3-b]quinolin-9-yl.

With respect to any relevant structural representation, such as Formula 1, in some embodiments, Ring B is:

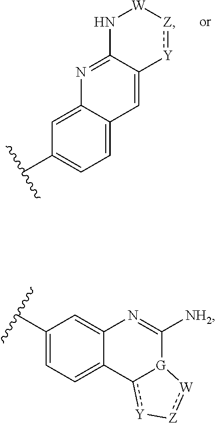

wherein each structure is optionally substituted; G is N or CR; the dashed line represents optionally with or without a bond; Y is —N(R^{A})—, N, C(R^{C}), —C(R^{C}R^{D})—, or —C(R^{C}R^{D})—C(R^{C}R^{D})—; Z is a bond, —N(R^{A})—, N, C(R^{C}), or —C(R^{C}R^{D})—; W is a bond, —N(R^{A})—, —O—, C(R^{C}), or —C(R^{C}R^{D})—, wherein each R^{C} and each R^{D} are independently H, F, Cl, Br, I, —NR^{A}R^{B}, C₁₋₆ hydrocarbyl, —OH, —CN, ═O, or —O—C₁₋₆ alkyl, and each R^{A} and each R^{B} are independently H, C₁₋₆ hydrocarbyl, C₁₋₆ heteroaryl, C₁₋₆ heterocycloalkyl, —C(O)—C₁₋₆ alkyl, —C(O)NH—C₁₋₆ alkyl, or —C(O)OC₁₋₆ alkyl; and wherein each R, each R^{A}, each R^{B}, each R^{C}, and each R^{D} are independently optionally halogenated.

In some embodiments, Ring B is represented by Formula 2 or 3:

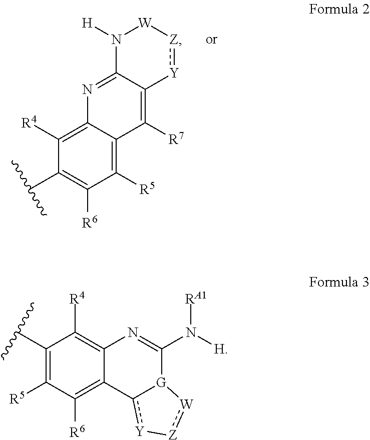

With respect to any relevant structural representation, such as Formula 2 or 3, the dashed line represents optionally with or without a bond.

With respect to any relevant structural representation, such as Formula 2, in some embodiments, Y and Z are linked by a single bond. In some embodiments, Y and Z are linked by a double bond. In some embodiments, YZ is —CH₂—CH₂—. In some embodiments, YZ is —C(CH₃)₂—CH₂—. In some embodiments, YZ is —CH₂—CH₂—CH₂—. In some embodiments, YZ is —CH═CH—. In some embodiments, YZ is —CH═CN—. In some embodiments, YZ is —CH═CN—, wherein Y is CH, and Z is CN.

With respect to any relevant structural representation, such as Formula 3, in some embodiments, W and Z are linked by a single bond. In some embodiments, Y and Z are linked by a double bond. In some embodiments, WZ is —CH₂—CH₂—. In some embodiments, WZ is —OCH₂—, In some embodiments, WZ is —CH═CH—. In some embodiments, WZ is —N═CH—.

With respect to any relevant structural representation, such as Formula 2 or 3, R⁴ is H or any substituent, such as R^{A}, F, Cl, CN, —OR^{A}, CF₃, —NO₂, —NR^{A}R^{B}, —COR^{A}, —CO₂R^{A}—OCOR^{A}, —NR^{A}COR^{B}, or —CONR^{A}R^{B}, etc. In some embodiments, R⁴ may be H, F, Cl, CN, CF₃, OH, NH₂, C₁₋₆ alkyl, or C₁₋₆ alkoxy. In some embodiments, R⁴ may be H, F, or Cl. In some embodiments, R⁴ may be H.

With respect to any relevant structural representation, such as Formula 2 or 3, R⁵ is H or any substituent, such as R^{A}, F, Cl, CN, —OR^{A}, CF₃, —NO₂, —NR^{A}R^{B}, —COR^{A}, CO₂R^{A}—OCOR^{A}, —NR^{A}COR^{B}, or —CONR^{A}R^{B}, etc. In some embodiments, R⁵ may be H, F, Cl, CN, CF₃, OH, NH₂, C₁₋₆ alkyl, or C₁₋₆ alkoxy. In some embodiments, R⁵ may be H, F, or C. In some embodiments, R⁵ may be H.

With respect to any relevant structural representation, such as Formula 2 or 3, R⁶ is H or any substituent, such as R^{A}, F, C, CN, —OR^{A}, CF₃, —NO₂, —NR^{A}R^{B}, —COR^{A}, —CO₂R^{A}, —OCOR^{A}, —NR^{A}COR^{B}, or —CONR^{A}R^{B}, etc. In some embodiments, R⁶ may be H, F, C, CN, CF₃, OH, NH₂, C₁₋₆ alkyl, or C₁₋₆ alkoxy. In some embodiments, R⁶ may be H, F, or C. In some embodiments, R⁶ may be H.

With respect to any relevant structural representation, such as Formula 2, R⁷ is H or any substituent, such as R^{A}, F, C, CN, —OR^{A}, CF₃, —NO₂, —NR^{A}R^{B}, —COR^{A}, —CO₂R^{A}, —OCOR^{A}, —NR^{A}COR^{B}, or —CONR^{A}R^{B}, etc. In some embodiments, R⁷ may be H, F, Cl, CN, CF₃, OH, NH₂, C₁₋₆ alkyl, or C₁₋₆ alkoxy. In some embodiments, R⁷ may be H, F, or Cl. In some embodiments, R⁷ may be H.

With respect to any relevant structural representation, such as Formula 2 or 3, R^{A1} is H or C₁₋₁₂ hydrocarbyl. In some embodiments, R^{A1} may be H or C₁₋₆ alkyl. In some embodiments, R^{A1} may be H or C₁₋₃ alkyl. In some embodiments, R^{A1} may be H or CH₃. In some embodiments, R^{A1} may be H.

With respect to any relevant structural representation, such as Formula 2 or 3, Y is —N(R^{A})— or —C(R^{C}R^{D})— In some embodiments, Y is —C(R^{C}R^{D})-. In some embodiments, Y is —CH—. In some embodiments, Y is —CH₂—. In some embodiments, Y is —C(CH₃)₂—. In some embodiments, Y is —C═O. In some embodiments, Y is —N(R^{A})—. In some embodiments, Y is —N-.

With respect to any relevant structural representation, such as Formula 2 or 3, Z is a bond, —N(R^{A})—, or —C(R^{C}R^{D})—. In some embodiments, Z is —C(R^{C}R^{D})—. In some embodiments, Z is —N(R^{A})—. In some embodiments, Z is —N-. In some embodiments, Z is —CH₂—. In some embodiments, Z is CH. In some embodiments, Z is a bond.

With respect to any relevant structural representation, such as Formula 2 or 3, W is a bond, —N(R^{A})—, N, —O—, C(R^{C}), or —C(R^{C}R^{D})—. In some embodiments, W is —C(R^{C}R^{D})—. In some embodiments, W is —N(R^{A})—. In some embodiments, W is N. In some embodiments, W is C(R^{C}).

In some embodiments, W is —CH—. In some embodiments, W is CH₂. In some embodiments, W is a bond. In some embodiments, W is —O-.

With respect to any relevant structural representation, such as Formula 3, G is N or CR. In some embodiments, G is N. In some embodiments, G is CR. In some embodiments, G is CH.

With respect to any relevant structural representation, such as Formula 2 or 3, in some embodiments, both W and Z are a bond, and G is CH.

With respect to any relevant structural representation, such as Formula 1, X is —O—, —CH₂—, or —CF₂—. In some embodiments, X is —CF₂—. In some embodiments, X is —O—. In some embodiments, X is —CH₂—.

With respect to any relevant structural representation, such as Formula 1, L is optionally substituted C₁₋₃ hydrocarbylene (e.g. —CH₂—, —C₂H₄, —CH═CH—, —C₃H₆-), optionally substituted —O—C₁₋₂ hydrocarbylene- (e.g. —O—CH₂—, —O-CH═CH—, —O—C₂H₄-, etc.), optionally substituted —S—C₁₋₂ hydrocarbylene-, or optionally substituted —NR^{A}—C₁₋₂ hydrocarbylene-. In some embodiments, L is C₁₋₃ hydrocarbylene. In some embodiments, L is —O—C₁₋₂ hydrocarbylene-. In some embodiments, L is —S—C₁₋₂ hydrocarbylene-. In some embodiments, L is —NR^{A}—C₁₋₂ hydrocarbylene-. In some embodiments, L is —CH₂—CH₂—CH₂—. In some embodiments, L is —CH₂—CH₂—.

Some embodiments include a compound represented by Formula 4 or 5 below:

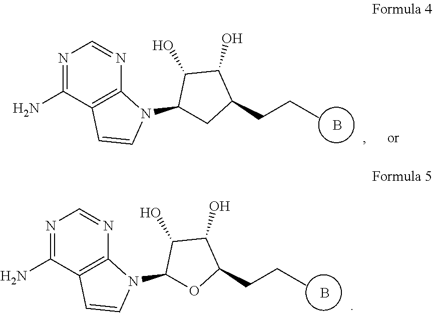

Formula 4 and 5 may be unsubstituted as shown, or the 4-amino-7H-pyrrolo[2,3-d]pyrimidin-7-yl may have 1, 2, or 3 substituents, such as those described elsewhere herein, or may be substituted at any position, such as those described elsewhere herein.

Some embodiments include optionally substituted (1S,2R,3S,5R)-3-(2-(2,3-dihydro-1H-pyrrolo[2,3-b]quinolin-7-yl)ethyl)-5-(7H-pyrrolo[2,3-d]pyrimidin-7-yl)cyclopentane-1,2-diol.

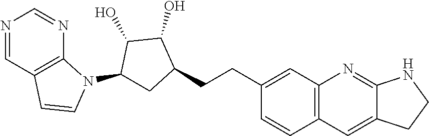 [SMILES: O[C@@H]1[C@@H](CCc2ccc3cc4c(nc3c2)NCC4)C[C@@H](n2ccc3cncnc32)[C@@H]1O]

Some embodiments include optionally substituted (1S,2R,3S,5R)-3-(2-(2,3-dihydroimidazo[1,2-c]quinazolin-8-yl)ethyl)-5-(7H-pyrrolo[2,3-d]pyrimidin-7-yl)cyclopentane-1,2-diol.

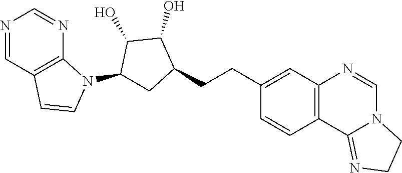 [SMILES: O[C@@H]1[C@@H](CCc2ccc3c(c2)N=CN2CCN=C32)C[C@@H](n2ccc3cncnc32)[C@@H]1O]

Some embodiments include optionally substituted (1S,2R,3S,5R)-3-(2-(imidazo[1,2-c]quinazolin-8-yl)ethyl)-5-(7H-pyrrolo[2,3-d]pyrimidin-7-yl)cyclopentane-1,2-diol.

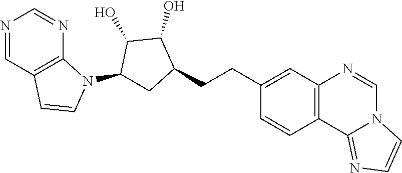 [SMILES: O[C@@H]1[C@@H](CCc2ccc3c(c2)ncn2ccnc32)C[C@@H](n2ccc3cncnc32)[C@@H]1O]

Some embodiments include optionally substituted (1S,2R,3S,5R)-3-(2-((1aS,7bR)-1a,7b-dihydro-1H-cyclopropa[c]quinolin-5-yl)ethyl)-5-(7H-pyrrolo[2,3-d]pyrimidin-7-yl)cyclopentane-1,2-diol.

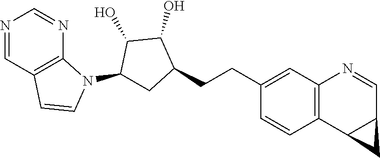 [SMILES: O[C@@H]1[C@@H](CCc2ccc3c(c2)N=C[C@H]2C[C@@H]32)C[C@@H](n2ccc3cncnc32)[C@@H]1O]

Some embodiments include optionally substituted (1S,2R,3S,5R)-3-(2-((1aR,7bS)-1a,7b-dihydro-1H-cyclopropa[c]quinolin-5-yl)ethyl)-5-(7H-pyrrolo[2,3-d]pyrimidin-7-yl)cyclopentane-1,2-diol.

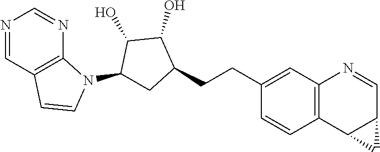 [SMILES: O[C@@H]1[C@@H](CCc2ccc3c(c2)N=C[C@@H]2C[C@H]32)C[C@@H](n2ccc3cncnc32)[C@@H]1O]

Some embodiments include optionally substituted (1S,2R,3S,5R)-3-(2-(1H-pyrazolo[3,4-b]quinolin-7-yl)ethyl)-5-(7H-pyrrolo[2,3-d]pyrimidin-7-yl)cyclopentane-1,2-diol.

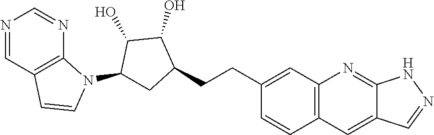 [SMILES: O[C@@H]1[C@@H](CCc2ccc3cc4cn[nH]c4nc3c2)C[C@@H](n2ccc3cncnc32)[C@@H]1O]

Some embodiments include optionally substituted (2R,3S,4R,5R)-2-(2-(2,3-dihydro-1H-pyrrolo[2,3-b]quinolin-7-yl)ethyl)-5-(7H-pyrrolo[2,3-d]pyrimidin-7-yl)tetrahydrofuran-3,4-diol.

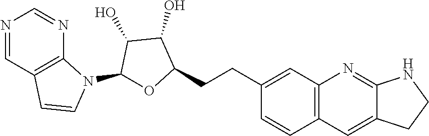 [SMILES: O[C@@H]1[C@H](O)[C@@H](CCc2ccc3cc4c(nc3c2)NCC4)O[C@H]1n1ccc2cncnc21]

Some embodiments include optionally substituted (2R,3S,4R,5R)-2-(2-(2,3-dihydroimidazo[1,2-c]quinazolin-8-yl)ethyl)-5-(7H-pyrrolo[2,3-d]pyrimidin-7-yl)tetrahydrofuran-3,4-diol.

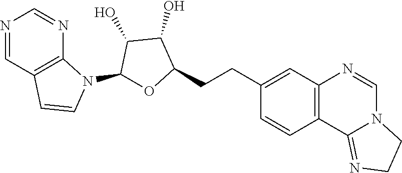 [SMILES: O[C@@H]1[C@H](O)[C@@H](CCc2ccc3c(c2)N=CN2CCN=C32)O[C@H]1n1ccc2cncnc21]

Some embodiments include optionally substituted (2R,3S,4R,5R)-2-(2-(imidazo[1,2-c]quinazolin-8-yl)ethyl)-5-(7H-pyrrolo[2,3-d]pyrimidin-7-yl)tetrahydrofuran-3,4-diol.

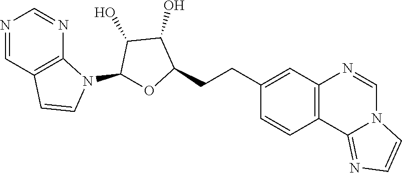 [SMILES: O[C@@H]1[C@H](O)[C@@H](CCc2ccc3c(c2)ncn2ccnc32)O[C@H]1n1ccc2cncnc21]

Some embodiments include optionally substituted (1S,2R,3S,5R)-3-(2-(1H-pyrrolo[2,3-b]quinolin-7-yl)ethyl)-5-(7H-pyrrolo[2,3-d]pyrimidin-7-yl)cyclopentane-1,2-diol.

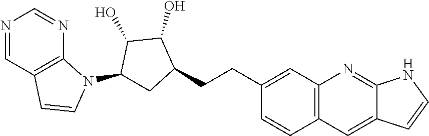 [SMILES: O[C@@H]1[C@@H](CCc2ccc3cc4cc[nH]c4nc3c2)C[C@@H](n2ccc3cncnc32)[C@@H]1O]

Some embodiments include optionally substituted (1S,2R,3S,5R)-3-(2-(3,4-dihydro-1H-[1,2]oxazino[3,4-b]quinolin-8-yl)ethyl)-5-(7H-pyrrolo[2,3-d]pyrimidin-7-yl)cyclopentane-1,2-diol.

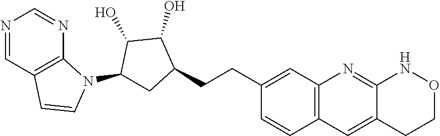 [SMILES: O[C@@H]1[C@@H](CCc2ccc3cc4c(nc3c2)NOCC4)C[C@@H](n2ccc3cncnc32)[C@@H]1O]

Some embodiments include optionally substituted (1S,2R,3S,5R)-3-(2-(1,3-dihydroisoxazolo[3,4-b]quinolin-7-yl)ethyl)-5-(7H-pyrrolo[2,3-d]pyrimidin-7-yl)cyclopentane-1,2-diol.

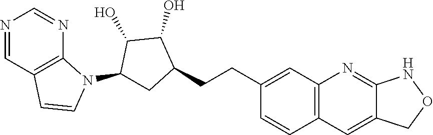 [SMILES: O[C@@H]1[C@@H](CCc2ccc3cc4c(nc3c2)NOC4)C[C@@H](n2ccc3cncnc32)[C@@H]1O]

Some embodiments include optionally substituted (1R,2S,3R,5S)-3-(7H-pyrrolo[2,3-d]pyrimidin-7-yl)-5-(2-(1,3,4,5-tetrahydro-[1,2]oxazepino[3,4-b]quinolin-9-yl)ethyl)cyclopentane-1,2-diol.

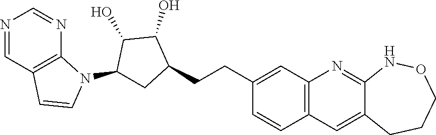 [SMILES: O[C@@H]1[C@@H](CCc2ccc3cc4c(nc3c2)NOCCC4)C[C@@H](n2ccc3cncnc32)[C@@H]1O]

Some embodiments include optionally substituted (1S,2R,3S,5R)-3-(2-(3H-imidazo[4,5-b]quinolin-6-yl)ethyl)-5-(7H-pyrrolo[2,3-d]pyrimidin-7-yl)cyclopentane-1,2-diol.

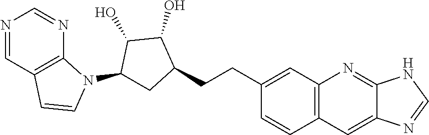 [SMILES: O[C@@H]1[C@@H](CCc2ccc3cc4nc[nH]c4nc3c2)C[C@@H](n2ccc3cncnc32)[C@@H]1O]

Some embodiments include optionally substituted (1S,2R,3S,5R)-3-(2-(3H-[1,2,3]triazolo[4,5-b]quinolin-6-yl)ethyl)-5-(7H-pyrrolo[2,3-d]pyrimidin-7-yl)cyclopentane-1,2-diol.

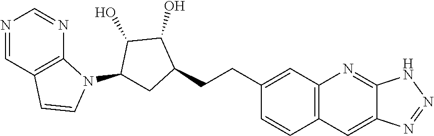 [SMILES: O[C@@H]1[C@@H](CCc2ccc3cc4nn[nH]c4nc3c2)C[C@@H](n2ccc3cncnc32)[C@@H]1O]

Some embodiments include optionally substituted (1S,2R,3S,5R)-3-(2-(2,3-dihydro-1H-pyrazolo[3,4-b]quinolin-7-yl)ethyl)-5-(7H-pyrrolo[2,3-d]pyrimidin-7-yl)cyclopentane-1,2-diol.

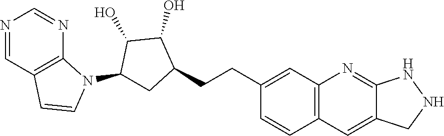 [SMILES: O[C@@H]1[C@@H](CCc2ccc3cc4c(nc3c2)NNC4)C[C@@H](n2ccc3cncnc32)[C@@H]1O]

Some embodiments include optionally substituted (1R,2S,3R,5S)-3-(7H-pyrrolo[2,3-d]pyrimidin-7-yl)-5-(2-(1,2,3,4-tetrahydrobenzo[b][1,8]naphthyridin-8-yl)ethyl)cyclopentane-1,2-diol.

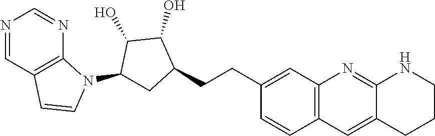 [SMILES: O[C@@H]1[C@@H](CCc2ccc3cc4c(nc3c2)NCCC4)C[C@@H](n2ccc3cncnc32)[C@@H]1O]

Some embodiments include optionally substituted (1R,2S,3R,5S)-3-(7H-pyrrolo[2,3-d]pyrimidin-7-yl)-5-(2-(2,3,4,5-tetrahydro-1H-azepino[2,3-b]quinolin-9-yl)ethyl)cyclopentane-1,2-diol.

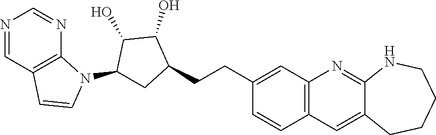 [SMILES: O[C@@H]1[C@@H](CCc2ccc3cc4c(nc3c2)NCCCC4)C[C@@H](n2ccc3cncnc32)[C@@H]1O]

Some embodiments include optionally substituted (2R,3S,4R,5R)-2-(2-(1H-pyrazolo[3,4-b]quinolin-7-yl)ethyl)-5-(7H-pyrrolo[2,3-d]pyrimidin-7-yl)tetrahydrofuran-3,4-diol.

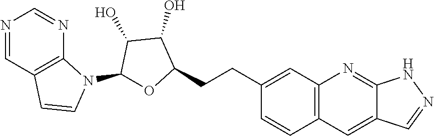 [SMILES: O[C@@H]1[C@H](O)[C@@H](CCc2ccc3cc4cn[nH]c4nc3c2)O[C@H]1n1ccc2cncnc21]

Some embodiments include optionally substituted (2R,3R,4S,5R)-2-(7H-pyrrolo[2,3-d]pyrimidin-7-yl)-5-(2-(1,2,3,4-tetrahydrobenzo[b][1,8]naphthyridin-8-yl)ethyl)tetrahydrofuran-3,4-diol.

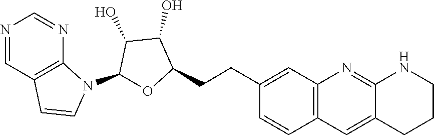 [SMILES: O[C@@H]1[C@H](O)[C@@H](CCc2ccc3cc4c(nc3c2)NCCC4)O[C@H]1n1ccc2cncnc21]

Some embodiments include optionally substituted (1R,2S,3R,5S)-3-(7H-pyrrolo[2,3-d]pyrimidin-7-yl)-5-(2-(1,2,3,4-tetrahydropyridazino[3,4-b]quinolin-8-yl)ethyl)cyclopentane-1,2-diol.

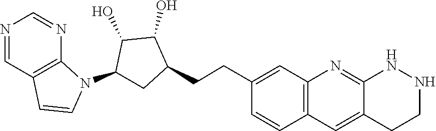 [SMILES: O[C@@H]1[C@@H](CCc2ccc3cc4c(nc3c2)NNCC4)C[C@@H](n2ccc3cncnc32)[C@@H]1O]

Some embodiments include one of the compounds listed below:

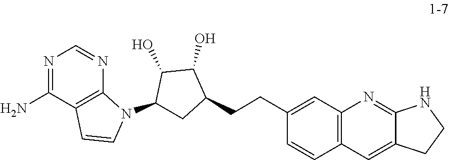 [SMILES: Nc1ncnc2c1ccn2[C@@H]1C[C@H](CCc2ccc3cc4c(nc3c2)NCC4)[C@@H](O)[C@H]1O]

(1R,2S,3R,5S)-3-(4-amino-7H-pyrrolo[2,3-d]pyrimidin-7-yl)-5-(2-(2,3-dihydro-1H-pyrrolo[2,3-b]quinolin-7-yl)ethyl)cyclopentane-1,2-diol,

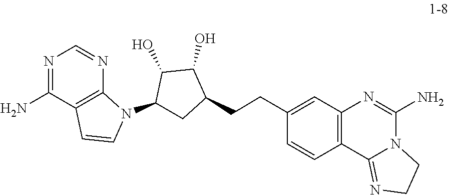 [SMILES: NC1=Nc2cc(CC[C@H]3C[C@@H](n4ccc5c(N)ncnc54)[C@H](O)[C@@H]3O)ccc2C2=NCCN12]

(1S,2R,3S,5R)-3-(2-(5-amino-2,3-dihydroimidazo[1,2-c]quinazolin-8-yl)ethyl)-5-(4-amino-7H-pyrrolo[2,3-d]pyrimidin-7-yl)cyclopentane-1,2-diol,

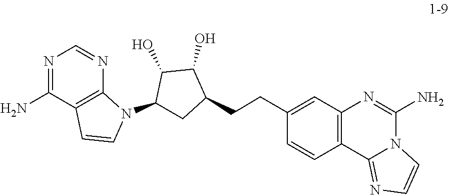 [SMILES: Nc1ncnc2c1ccn2[C@@H]1C[C@H](CCc2ccc3c(c2)nc(N)n2ccnc32)[C@@H](O)[C@H]1O]

(1R,2S,3R,5S)-3-(4-amino-7H-pyrrolo[2,3-d]pyrimidin-7-yl)-5-(2-(5-aminoimidazo-[1,2-c]quinazolin-8-yl)ethyl)cyclopentane-1,2-diol,

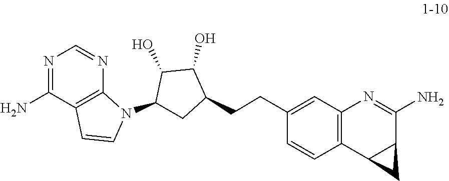 [SMILES: NC1=Nc2cc(CC[C@H]3C[C@@H](n4ccc5c(N)ncnc54)[C@H](O)[C@@H]3O)ccc2[C@@H]2C[C@H]12]

(1S,2R,3S,5R)-3-(2-((1aS,7bR)-2-amino-1a,7b-dihydro-1H-cyclopropa[c]quinolin-5-yl)-ethyl)-5-(4-amino-7H-pyrrolo[2,3-d]pyrimidin-7-yl)cyclopentane-1,2-diol

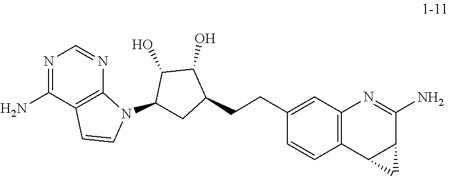 [SMILES: NC1=Nc2cc(CC[C@H]3C[C@@H](n4ccc5c(N)ncnc54)[C@H](O)[C@@H]3O)ccc2[C@H]2C[C@@H]12]

(1S,2R,3S,5R)-3-(2-((1aR,7bS)-2-amino-1a,7b-dihydro-1H-cyclopropa[c]quinolin-5-yl)-ethyl)-5-(4-amino-7H-pyrrolo[2,3-d]pyrimidin-7-yl)cyclopentane-1,2-diol,

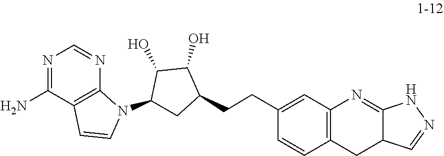 [SMILES: Nc1ncnc2c1ccn2[C@@H]1C[C@H](CCc2ccc3c(c2)N=C2NN=CC2C3)[C@@H](O)[C@H]1O]

(1S,2R,3S,5R)-3-(2-(1H-pyrazolo[3,4-b]quinolin-7-yl)ethyl)-5-(4-amino-7H-pyrrolo-[2,3-d]pyrimidin-7-yl)cyclopentane-1,2-diol,

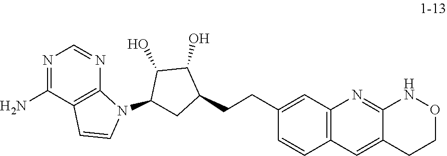 [SMILES: Nc1ncnc2c1ccn2[C@@H]1C[C@H](CCc2ccc3cc4c(nc3c2)NOCC4)[C@@H](O)[C@H]1O]

(1R,2S,3R,5S)-3-(4-amino-7H-pyrrolo[2,3-d]pyrimidin-7-yl)-5-(2-(3,4-dihydro-1H-[1,2]oxazino[3,4-b]quinolin-8-yl)ethyl)cyclopentane-1,2-diol,

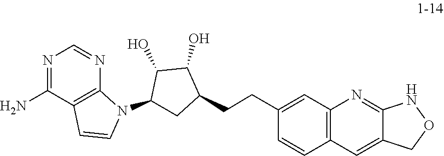 [SMILES: Nc1ncnc2c1ccn2[C@@H]1C[C@H](CCc2ccc3cc4c(nc3c2)NOC4)[C@@H](O)[C@H]1O]

(1R,2S,3R,5S)-3-(4-amino-7H-pyrrolo[2,3-d]pyrimidin-7-yl)-5-(2-(1,3-dihydroisoxazolo[3,4-b]quinolin-7-yl)ethyl)cyclopentane-1,2-diol,

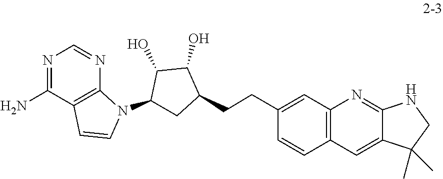 [SMILES: CC1(C)CNc2nc3cc(CC[C@H]4C[C@@H](n5ccc6c(N)ncnc65)[C@H](O)[C@@H]4O)ccc3cc21]

(1R,2S,3R,5S)-3-(4-amino-7H-pyrrolo[2,3-d]pyrimidin-7-yl)-5-(2-(3,3-dimethyl-2,3-dihydro-1H-pyrrolo[2,3-b]quinolin-7-yl)ethyl)cyclopentane-1,2-diol

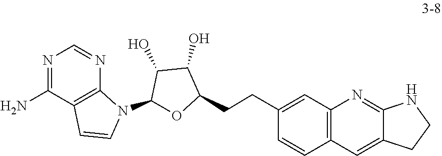 [SMILES: Nc1ncnc2c1ccn2[C@@H]1O[C@H](CCc2ccc3cc4c(nc3c2)NCC4)[C@@H](O)[C@H]1O]

(2R,3R,4S,5R)-2-(4-amino-7H-pyrrolo[2,3-d]pyrimidin-7-yl)-5-(2-(2,3-dihydro-1H-pyrrolo[2,3-b]quinolin-7-yl)ethyl)tetrahydrofuran-3,4-diol,

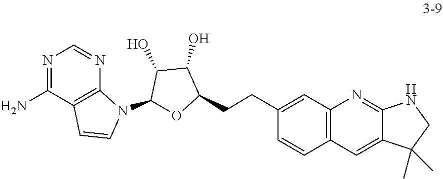 [SMILES: CC1(C)CNc2nc3cc(CC[C@H]4O[C@@H](n5ccc6c(N)ncnc65)[C@H](O)[C@@H]4O)ccc3cc21]

(2R,3R,4S,5R)-2-(4-amino-7H-pyrrolo[2,3-d]pyrimidin-7-yl)-5-(2-(3,3-dimethyl-2,3-dihydro-1H-pyrrolo[2,3-b]quinolin-7-yl)ethyl)tetrahydrofuran-3,4-diol,

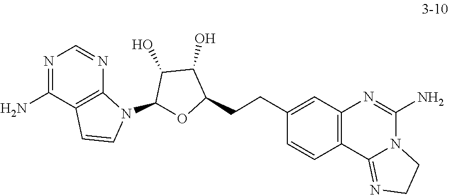 [SMILES: NC1=Nc2cc(CC[C@H]3O[C@@H](n4ccc5c(N)ncnc54)[C@H](O)[C@@H]3O)ccc2C2=NCCN12]

(2R,3S,4R,5R)-2-(2-(5-amino-2,3-dihydroimidazo[1,2-c]quinazolin-8-yl)ethyl)-5-(4-amino-7H-pyrrolo[2,3-d]pyrimidin-7-yl)tetrahydrofuran-3,4-diol,

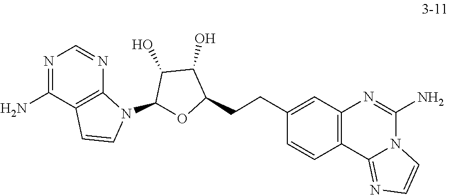 [SMILES: Nc1ncnc2c1ccn2[C@@H]1O[C@H](CCc2ccc3c(c2)nc(N)n2ccnc32)[C@@H](O)[C@H]1O]

(2R,3R,4S,5R)-2-(4-amino-7H-pyrrolo[2,3-d]pyrimidin-7-yl)-5-(2-(5-aminoimidazo-[1,2-c]quinazolin-8-yl)ethyl)tetrahydrofuran-3,4-diol,

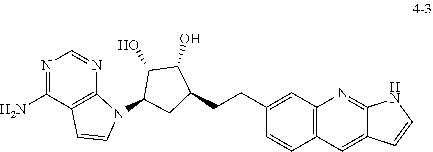 [SMILES: Nc1ncnc2c1ccn2[C@@H]1C[C@H](CCc2ccc3cc4cc[nH]c4nc3c2)[C@@H](O)[C@H]1O]

(1S,2R,3S,5R)-3-(2-(1H-pyrrolo[2,3-b]quinolin-7-yl)ethyl)-5-(4-amino-7H-pyrrolo[2,3-d]pyrimidin-7-yl)cyclopentane-1,2-diol

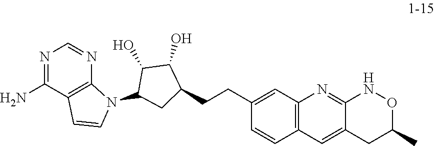

(1R,2S,3R,5S)-3-(4-amino-7H-pyrrolo[2,3-d]pyrimidin-7-yl)-5-(2-((S)-3-methyl-3,4-dihydro-1H-[1,2]oxazino[3,4-b]quinolin-8-yl)ethyl)cyclopentane-1,2-diol,

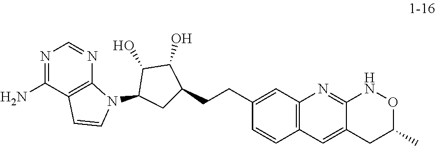 [SMILES: C[C@@H]1Cc2cc3ccc(CC[C@H]4C[C@@H](n5ccc6c(N)ncnc65)[C@H](O)[C@@H]4O)cc3nc2NO1]

(1R,2S,3R,5S)-3-(4-amino-7H-pyrrolo[2,3-d]pyrimidin-7-yl)-5-(2-((R)-3-methyl-3,4-dihydro-1H-[1,2]oxazino[3,4-b]quinolin-8-yl)ethyl)cyclopentane-1,2-diol,

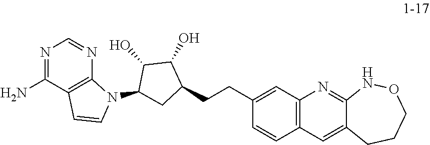 [SMILES: Nc1ncnc2c1ccn2[C@@H]1C[C@H](CCc2ccc3cc4c(nc3c2)NOCCC4)[C@@H](O)[C@H]1O]

(1R,2S,3R,5S)-3-(4-amino-7H-pyrrolo[2,3-d]pyrimidin-7-yl)-5-(2-(1,3,4,5-tetrahydro-[1,2]oxazepino[3,4-b]quinolin-9-yl)ethyl)cyclopentane-1,2-diol,

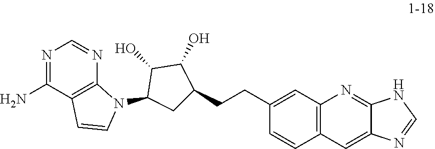 [SMILES: Nc1ncnc2c1ccn2[C@@H]1C[C@H](CCc2ccc3cc4nc[nH]c4nc3c2)[C@@H](O)[C@H]1O]

(1S,2R,3S,5R)-3-(2-(3H-imidazo[4,5-b]quinolin-6-yl)ethyl)-5-(4-amino-7H-pyrrolo[2,3-d]pyrimidin-7-yl)cyclopentane-1,2-diol,

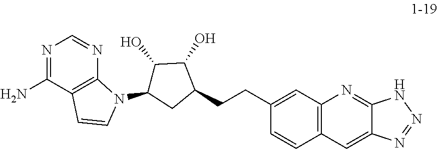 [SMILES: Nc1ncnc2c1ccn2[C@@H]1C[C@H](CCc2ccc3cc4nn[nH]c4nc3c2)[C@@H](O)[C@H]1O]

(1S,2R,3S,5R)-3-(2-(3H-[1,2,3]triazolo[4,5-b]quinolin-6-yl)ethyl)-5-(4-amino-7H-pyrrolo[2,3-d]pyrimidin-7-yl)cyclopentane-1,2-diol

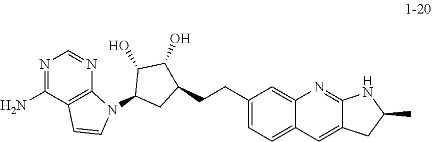 [SMILES: C[C@H]1Cc2cc3ccc(CC[C@H]4C[C@@H](n5ccc6c(N)ncnc65)[C@H](O)[C@@H]4O)cc3nc2N1]

(1R,2S,3R,5S)-3-(4-amino-7H-pyrrolo[2,3-d]pyrimidin-7-yl)-5-(2-((S)-2-methyl-2,3-dihydro-1H-pyrrolo[2,3-b]quinolin-7-yl)ethyl)cyclopentane-1,2-diol,

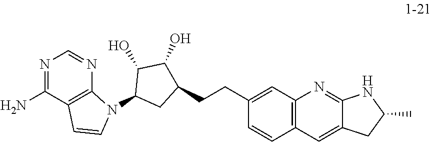 [SMILES: C[C@@H]1Cc2cc3ccc(CC[C@H]4C[C@@H](n5ccc6c(N)ncnc65)[C@H](O)[C@@H]4O)cc3nc2N1]

(1R,2S,3R,5S)-3-(4-amino-7H-pyrrolo[2,3-d]pyrimidin-7-yl)-5-(2-((R)-2-methyl-2,3-dihydro-1H-pyrrolo[2,3-b]quinolin-7-yl)ethyl)cyclopentane-1,2-diol,

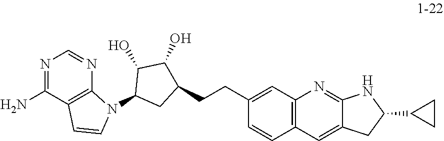 [SMILES: Nc1ncnc2c1ccn2[C@@H]1C[C@H](CCc2ccc3cc4c(nc3c2)N[C@H](C2CC2)C4)[C@@H](O)[C@H]1O]

(1R,2S,3R,5S)-3-(4-amino-7H-pyrrolo[2,3-d]pyrimidin-7-yl)-5-(2-((S)-2-cyclopropyl-2,3-dihydro-1H-pyrrolo[2,3-b]quinolin-7-yl)ethyl)cyclopentane-1,2-diol,

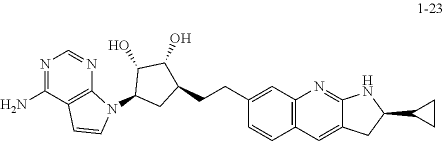 [SMILES: Nc1ncnc2c1ccn2[C@@H]1C[C@H](CCc2ccc3cc4c(nc3c2)N[C@@H](C2CC2)C4)[C@@H](O)[C@H]1O]

(1R,2S,3R,5S)-3-(4-amino-7H-pyrrolo[2,3-d]pyrimidin-7-yl)-5-(2-((R)-2-cyclopropyl-2,3-dihydro-1H-pyrrolo[2,3-b]quinolin-7-yl)ethyl)cyclopentane-1,2-diol,

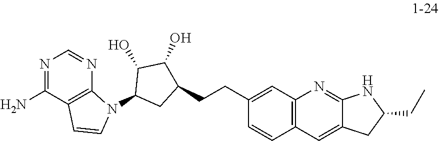 [SMILES: CC[C@@H]1Cc2cc3ccc(CC[C@H]4C[C@@H](n5ccc6c(N)ncnc65)[C@H](O)[C@@H]4O)cc3nc2N1]

(1R,2S,3R,5S)-3-(4-amino-7H-pyrrolo[2,3-d]pyrimidin-7-yl)-5-(2-((R)-2-ethyl-2,3-dihydro-1H-pyrrolo[2,3-b]quinolin-7-yl)ethyl)cyclopentane-1,2-diol,

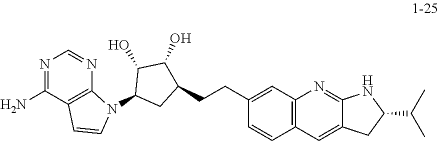 [SMILES: CC(C)[C@@H]1Cc2cc3ccc(CC[C@H]4C[C@@H](n5ccc6c(N)ncnc65)[C@H](O)[C@@H]4O)cc3nc2N1]

(1R,2S,3R,5S)-3-(4-amino-7H-pyrrolo[2,3-d]pyrimidin-7-yl)-5-(2-((S)-2-isopropyl-2,3-dihydro-1H-pyrrolo[2,3-b]quinolin-7-yl)ethyl)cyclopentane-1,2-diol,

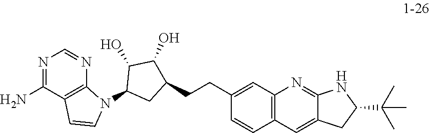 [SMILES: CC(C)(C)[C@@H]1Cc2cc3ccc(CC[C@H]4C[C@@H](n5ccc6c(N)ncnc65)[C@H](O)[C@@H]4O)cc3nc2N1]

(1R,2S,3R,5S)-3-(4-amino-7H-pyrrolo[2,3-d]pyrimidin-7-yl)-5-(2-((S)-2-(tert-butyl)-2,3-dihydro-1H-pyrrolo[2,3-b]quinolin-7-yl)ethyl)cyclopentane-1,2-diol,

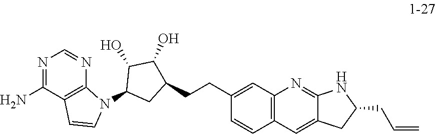 [SMILES: C=CC[C@@H]1Cc2cc3ccc(CC[C@H]4C[C@@H](n5ccc6c(N)ncnc65)[C@H](O)[C@@H]4O)cc3nc2N1]

(1S,2R,3S,5R)-3-(2-((R)-2-allyl-2,3-dihydro-1H-pyrrolo[2,3-b]quinolin-7-yl)ethyl)-5-(4-amino-7H-pyrrolo[2,3-d]pyrimidin-7-yl)cyclopentane-1,2-diol,

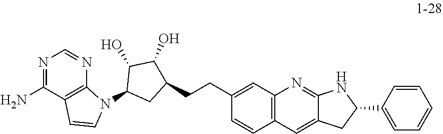 [SMILES: Nc1ncnc2c1ccn2[C@@H]1C[C@H](CCc2ccc3cc4c(nc3c2)N[C@H](c2ccccc2)C4)[C@@H](O)[C@H]1O]

(1R,2S,3R,5S)-3-(4-amino-7H-pyrrolo[2,3-d]pyrimidin-7-yl)-5-(2-((S)-2-phenyl-2,3-dihydro-1H-pyrrolo[2,3-b]quinolin-7-yl)ethyl)cyclopentane-1,2-diol,

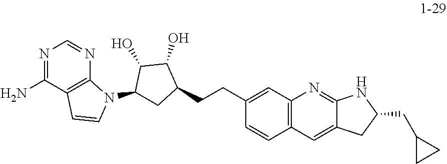 [SMILES: Nc1ncnc2c1ccn2[C@@H]1C[C@H](CCc2ccc3cc4c(nc3c2)N[C@H](CC2CC2)C4)[C@@H](O)[C@H]1O]

(1R,2S,3R,5S)-3-(4-amino-7H-pyrrolo[2,3-d]pyrimidin-7-yl)-5-(2-((R)-2-(cyclopropylmethyl)-2,3-dihydro-1H-pyrrolo[2,3-b]quinolin-7-yl)ethyl)cyclopentane-1,2-diol,

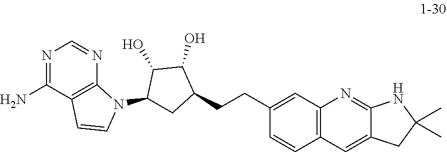 [SMILES: CC1(C)Cc2cc3ccc(CC[C@H]4C[C@@H](n5ccc6c(N)ncnc65)[C@H](O)[C@@H]4O)cc3nc2N1]

(1R,2S,3R,5S)-3-(4-amino-7H-pyrrolo[2,3-d]pyrimidin-7-yl)-5-(2-(2,2-dimethyl-2,3-dihydro-1H-pyrrolo[2,3-b]quinolin-7-yl)ethyl)cyclopentane-1,2-diol,

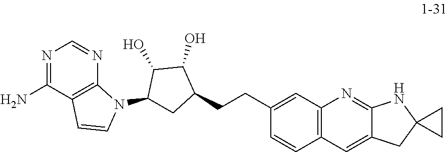 [SMILES: Nc1ncnc2c1ccn2[C@@H]1C[C@H](CCc2ccc3cc4c(nc3c2)NC2(CC2)C4)[C@@H](O)[C@H]1O]

(1R,2S,3R,5S)-3-(4-amino-7H-pyrrolo[2,3-d]pyrimidin-7-yl)-5-(2-(1′,3′-dihydrospiro[cyclopropane-1,2′-pyrrolo[2,3-b]quinolin]-7′-yl)ethyl)cyclopentane-1,2-diol,

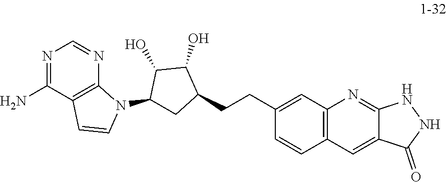 [SMILES: Nc1ncnc2c1ccn2[C@@H]1C[C@H](CCc2ccc3cc4c(=O)[nH][nH]c4nc3c2)[C@@H](O)[C@H]1O]

7-(2-((1S,2R,3S,4R)-4-(4-amino-7H-pyrrolo[2,3-d]pyrimidin-7-yl)-2,3-dihydroxycyclopentyl)ethyl)-1,2-dihydro-3H-pyrazolo[3,4-b]quinolin-3-one,

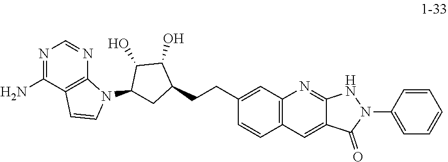 [SMILES: Nc1ncnc2c1ccn2[C@@H]1C[C@H](CCc2ccc3cc4c(=O)n(-c5ccccc5)[nH]c4nc3c2)[C@@H](O)[C@H]1O]

7-(2-((1S,2R,3S,4R)-4-(4-amino-7H-pyrrolo[2,3-d]pyrimidin-7-yl)-2,3-dihydroxycyclopentyl)ethyl)-2-phenyl-1,2-dihydro-3H-pyrazolo[3,4-b]quinolin-3-one,

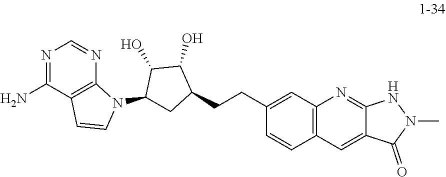 [SMILES: Cn1[nH]c2nc3cc(CC[C@H]4C[C@@H](n5ccc6c(N)ncnc65)[C@H](O)[C@@H]4O)ccc3cc2c1=O]

7-(2-((1S,2R,3S,4R)-4-(4-amino-7H-pyrrolo[2,3-d]pyrimidin-7-yl)-2,3-dihydroxycyclopentyl)ethyl)-2-methyl-1,2-dihydro-3H-pyrazolo[3,4-b]quinolin-3-one,

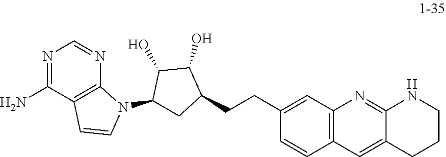 [SMILES: Nc1ncnc2c1ccn2[C@@H]1C[C@H](CCc2ccc3cc4c(nc3c2)NCCC4)[C@@H](O)[C@H]1O]

(1R,2S,3R,5S)-3-(4-amino-7H-pyrrolo[2,3-d]pyrimidin-7-yl)-5-(2-(1,2,3,4-tetrahydrobenzo[b][1,8]naphthyridin-8-yl)ethyl)cyclopentane-1,2-diol,

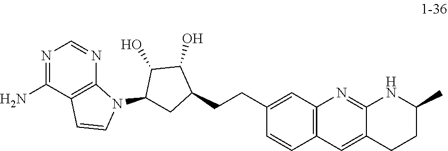 [SMILES: C[C@H]1CCc2cc3ccc(CC[C@H]4C[C@@H](n5ccc6c(N)ncnc65)[C@H](O)[C@@H]4O)cc3nc2N1]

(1R,2S,3R,5S)-3-(4-amino-7H-pyrrolo[2,3-d]pyrimidin-7-yl)-5-(2-((S)-2-methyl-1,2,3,4-tetrahydrobenzo[b][1,8]naphthyridin-8-yl)ethyl)cyclopentane-1,2-diol,

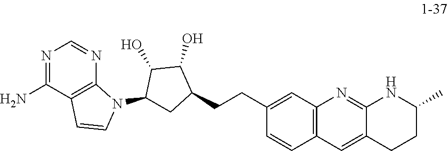 [SMILES: C[C@@H]1CCc2cc3ccc(CC[C@H]4C[C@@H](n5ccc6c(N)ncnc65)[C@H](O)[C@@H]4O)cc3nc2N1]

(1R,2S,3R,5S)-3-(4-amino-7H-pyrrolo[2,3-d]pyrimidin-7-yl)-5-(2-((R)-2-methyl-1,2,3,4-tetrahydrobenzo[b][1,8]naphthyridin-8-yl)ethyl)cyclopentane-1,2-diol,

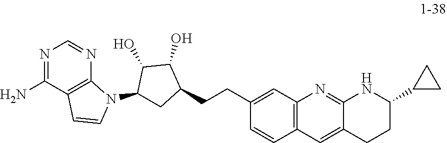 [SMILES: Nc1ncnc2c1ccn2[C@@H]1C[C@H](CCc2ccc3cc4c(nc3c2)N[C@H](C2CC2)CC4)[C@@H](O)[C@H]1O]

(1R,2S,3R,5S)-3-(4-amino-7H-pyrrolo[2,3-d]pyrimidin-7-yl)-5-(2-((S)-2-cyclopropyl-1,2,3,4-tetrahydrobenzo[b][1,8]naphthyridin-8-yl)ethyl)cyclopentane-1,2-diol,

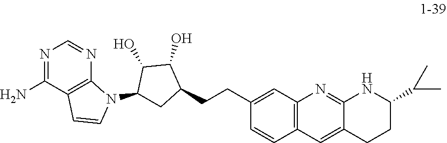 [SMILES: CC(C)[C@@H]1CCc2cc3ccc(CC[C@H]4C[C@@H](n5ccc6c(N)ncnc65)[C@H](O)[C@@H]4O)cc3nc2N1]

(1R,2S,3R,5S)-3-(4-amino-7H-pyrrolo[2,3-d]pyrimidin-7-yl)-5-(2-((S)-2-isopropyl-1,2,3,4-tetrahydrobenzo[b][1,8]naphthyridin-8-yl)ethyl)cyclopentane-1,2-diol,

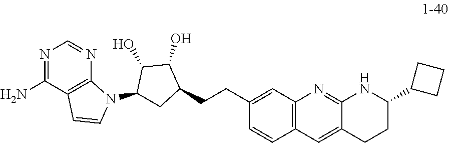 [SMILES: Nc1ncnc2c1ccn2[C@@H]1C[C@H](CCc2ccc3cc4c(nc3c2)N[C@H](C2CCC2)CC4)[C@@H](O)[C@H]1O]

(1R,2S,3R,5S)-3-(4-amino-7H-pyrrolo[2,3-d]pyrimidin-7-yl)-5-(2-((S)-2-cyclobutyl-1,2,3,4-tetrahydrobenzo[b][1,8]naphthyridin-8-yl)ethyl)cyclopentane-1,2-diol,

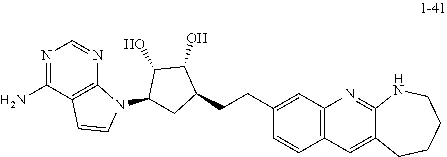 [SMILES: Nc1ncnc2c1ccn2[C@@H]1C[C@H](CCc2ccc3cc4c(nc3c2)NCCCC4)[C@@H](O)[C@H]1O]

(1R,2S,3R,5S)-3-(4-amino-7H-pyrrolo[2,3-d]pyrimidin-7-yl)-5-(2-(2,3,4,5-tetrahydro-1H-azepino[2,3-b]quinolin-9-yl)ethyl)cyclopentane-1,2-diol,

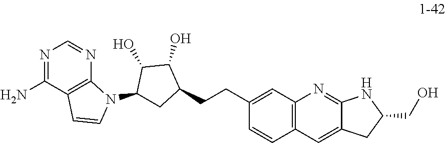 [SMILES: Nc1ncnc2c1ccn2[C@@H]1C[C@H](CCc2ccc3cc4c(nc3c2)N[C@H](CO)C4)[C@@H](O)[C@H]1O]

(1R,2S,3R,5S)-3-(4-amino-7H-pyrrolo[2,3-d]pyrimidin-7-yl)-5-(2-((S)-2-(hydroxymethyl)-2,3-dihydro-1H-pyrrolo[2,3-b]quinolin-7-yl)ethyl)cyclopentane-1,2-diol,

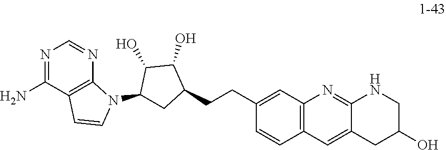 [SMILES: Nc1ncnc2c1ccn2[C@@H]1C[C@H](CCc2ccc3cc4c(nc3c2)NCC(O)C4)[C@@H](O)[C@H]1O]

(1R,2S,3R,5S)-3-(4-amino-7H-pyrrolo[2,3-d]pyrimidin-7-yl)-5-(2-(3-hydroxy-1,2,3,4-tetrahydrobenzo[b][1,8]naphthyridin-8-yl)ethyl)cyclopentane-1,2-diol,

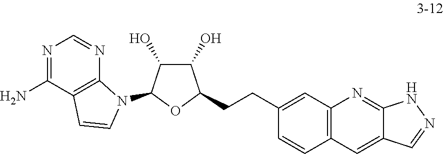 [SMILES: Nc1ncnc2c1ccn2[C@@H]1O[C@H](CCc2ccc3cc4cn[nH]c4nc3c2)[C@@H](O)[C@H]1O]

(2R,3S,4R,5R)-2-(2-(1H-pyrazolo[3,4-b]quinolin-7-yl)ethyl)-5-(4-amino-7H-pyrrolo[2,3-d]pyrimidin-7-yl)tetrahydrofuran-3,4-diol,

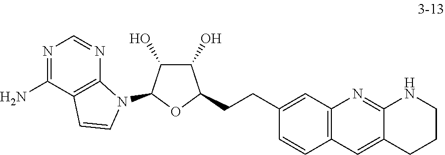 [SMILES: Nc1ncnc2c1ccn2[C@@H]1O[C@H](CCc2ccc3cc4c(nc3c2)NCCC4)[C@@H](O)[C@H]1O]

(2R,3R,4S,5R)-2-(4-amino-7H-pyrrolo[2,3-d]pyrimidin-7-yl)-5-(2-(1,2,3,4-tetrahydrobenzo[b][1,8]naphthyridin-8-yl)ethyl)tetrahydrofuran-3,4-diol,

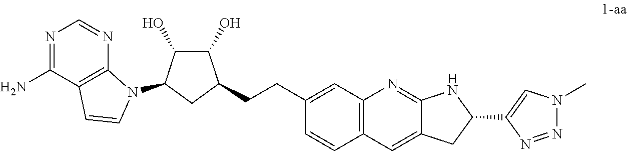 [SMILES: Cn1cc([C@@H]2Cc3cc4ccc(CC[C@H]5C[C@@H](n6ccc7c(N)ncnc76)[C@H](O)[C@@H]5O)cc4nc3N2)nn1]

(1R,2S,3R,5S)-3-(4-amino-7H-pyrrolo[2,3-d]pyrimidin-7-yl)-5-(2-((S)-2-(1-methyl-1H-1,2,3-triazol-4-yl)-2,3-dihydro-1H-pyrrolo[2,3-b]quinolin-7-yl)ethyl)cyclopentane-1,2-diol,

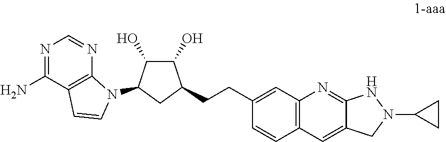 [SMILES: Nc1ncnc2c1ccn2[C@@H]1C[C@H](CCc2ccc3cc4c(nc3c2)NN(C2CC2)C4)[C@@H](O)[C@H]1O]

(1R,2S,3R,5S)-3-(4-amino-7H-pyrrolo[2,3-d]pyrimidin-7-yl)-5-(2-(2-cyclopropyl-2,3-dihydro-1H-pyrazolo[3,4-b]quinolin-7-yl)ethyl)cyclopentane-1,2-diol, or

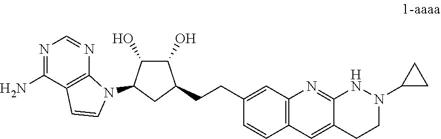 [SMILES: Nc1ncnc2c1ccn2[C@@H]1C[C@H](CCc2ccc3cc4c(nc3c2)NN(C2CC2)CC4)[C@@H](O)[C@H]1O]

(1R,2S,3R,5S)-3-(4-amino-7H-pyrrolo[2,3-d]pyrimidin-7-yl)-5-(2-(2-cyclopropyl-1,2,3,4-tetrahydropyridazino[3,4-b]quinolin-8-yl)ethyl)cyclopentane-1,2-diol.

Some embodiments include one of the compounds listed in Table 1 below, wherein each structure can be optionally substituted.

TABLE 1
Compound structures and their identification numbers
Compound Structure and ID Number

| | |
|---|---|
| 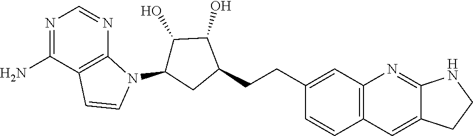 [SMILES: Nc1ncnc2c1ccn2[C@@H]1C[C@H](CCc2ccc3cc4c(nc3c2)NCC4)[C@@H](O)[C@H]1O] | 1-7 |
| 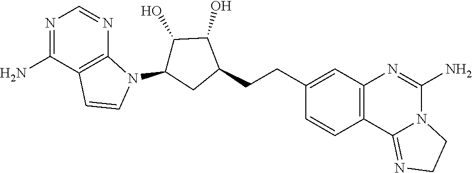 [SMILES: NC1=Nc2cc(CC[C@H]3C[C@@H](n4ccc5c(N)ncnc54)[C@H](O)[C@@H]3O)ccc2C2=NCCN12] | 1-8 |
| 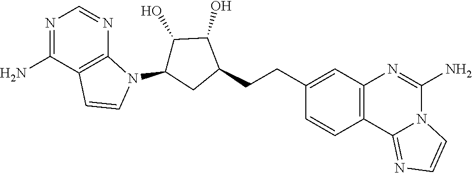 [SMILES: Nc1ncnc2c1ccn2[C@@H]1C[C@H](CCc2ccc3c(c2)nc(N)n2ccnc32)[C@@H](O)[C@H]1O] | 1-9 |
| 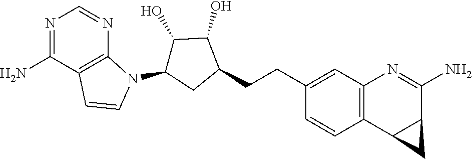 [SMILES: NC1=Nc2cc(CC[C@H]3C[C@@H](n4ccc5c(N)ncnc54)[C@H](O)[C@@H]3O)ccc2[C@@H]2C[C@H]12] | 1-10 |
| 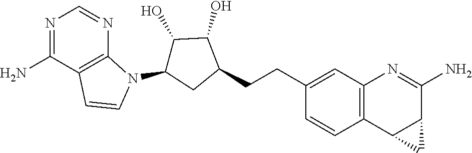 [SMILES: NC1=Nc2cc(CC[C@H]3C[C@@H](n4ccc5c(N)ncnc54)[C@H](O)[C@@H]3O)ccc2[C@H]2C[C@@H]12] | 1-11 |
| 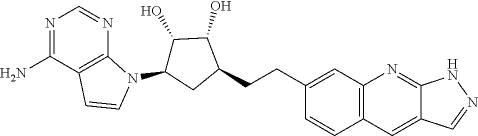 [SMILES: Nc1ncnc2c1ccn2[C@@H]1C[C@H](CCc2ccc3cc4cn[nH]c4nc3c2)[C@@H](O)[C@H]1O] | 1-12 |
| 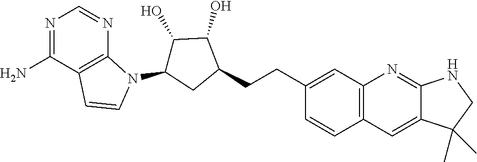 [SMILES: CC1(C)CNc2nc3cc(CC[C@H]4C[C@@H](n5ccc6c(N)ncnc65)[C@H](O)[C@@H]4O)ccc3cc21] | 2-3 |
| 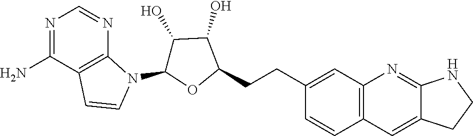 [SMILES: Nc1ncnc2c1ccn2[C@@H]1O[C@H](CCc2ccc3cc4c(nc3c2)NCC4)[C@@H](O)[C@H]1O] | 3-8 |
| 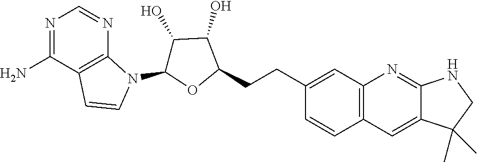 [SMILES: CC1(C)CNc2nc3cc(CC[C@H]4O[C@@H](n5ccc6c(N)ncnc65)[C@H](O)[C@@H]4O)ccc3cc21] | 3-9 |
| 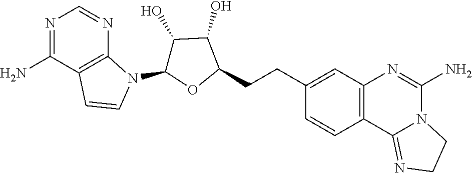 [SMILES: NC1=Nc2cc(CC[C@H]3O[C@@H](n4ccc5c(N)ncnc54)[C@H](O)[C@@H]3O)ccc2C2=NCCN12] | 3-10 |
| 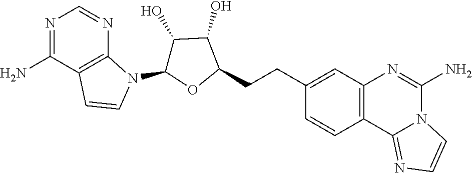 [SMILES: Nc1ncnc2c1ccn2[C@@H]1O[C@H](CCc2ccc3c(c2)nc(N)n2ccnc32)[C@@H](O)[C@H]1O] | 3-11 |
| 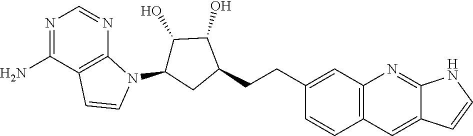 [SMILES: Nc1ncnc2c1ccn2[C@@H]1C[C@H](CCc2ccc3cc4cc[nH]c4nc3c2)[C@@H](O)[C@H]1O] | 4-3 |
| 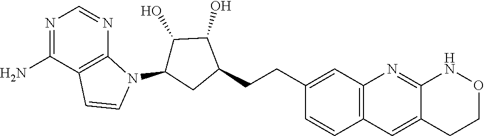 [SMILES: Nc1ncnc2c1ccn2[C@@H]1C[C@H](CCc2ccc3cc4c(nc3c2)NOCC4)[C@@H](O)[C@H]1O] | 1-13 |
| 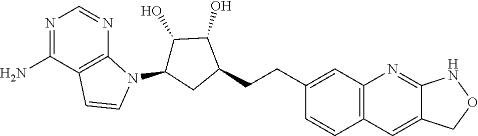 [SMILES: Nc1ncnc2c1ccn2[C@@H]1C[C@H](CCc2ccc3cc4c(nc3c2)NOC4)[C@@H](O)[C@H]1O] | 1-14 |
| 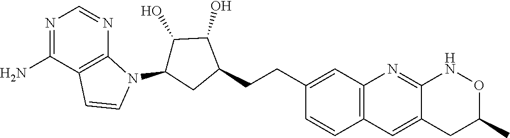 [SMILES: C[C@H]1Cc2cc3ccc(CC[C@H]4C[C@@H](n5ccc6c(N)ncnc65)[C@H](O)[C@@H]4O)cc3nc2NO1] | 1-15 |
| 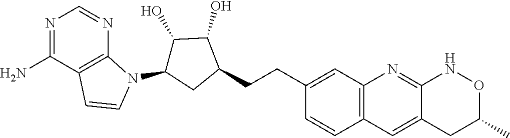 [SMILES: C[C@@H]1Cc2cc3ccc(CC[C@H]4C[C@@H](n5ccc6c(N)ncnc65)[C@H](O)[C@@H]4O)cc3nc2NO1] | 1-16 |
| 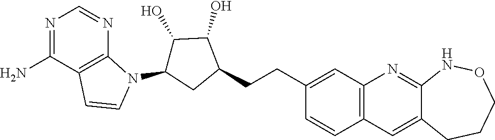 [SMILES: Nc1ncnc2c1ccn2[C@@H]1C[C@H](CCc2ccc3cc4c(nc3c2)NOCCC4)[C@@H](O)[C@H]1O] | 1-17 |
| 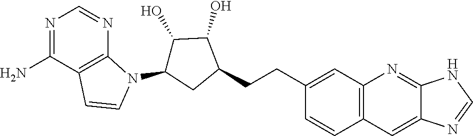 [SMILES: Nc1ncnc2c1ccn2[C@@H]1C[C@H](CCc2ccc3cc4nc[nH]c4nc3c2)[C@@H](O)[C@H]1O] | 1-18 |
| 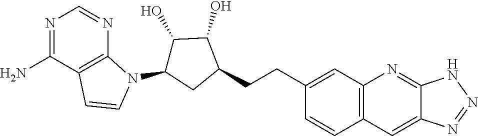 [SMILES: Nc1ncnc2c1ccn2[C@@H]1C[C@H](CCc2ccc3cc4nn[nH]c4nc3c2)[C@@H](O)[C@H]1O] | 1-19 |
| 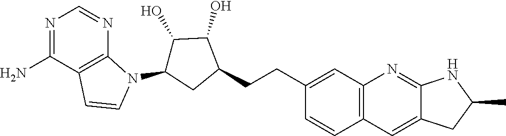 [SMILES: C[C@H]1Cc2cc3ccc(CC[C@H]4C[C@@H](n5ccc6c(N)ncnc65)[C@H](O)[C@@H]4O)cc3nc2N1] | 1-20 |
| 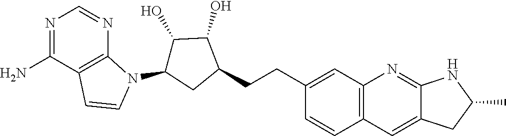 [SMILES: C[C@@H]1Cc2cc3ccc(CC[C@H]4C[C@@H](n5ccc6c(N)ncnc65)[C@H](O)[C@@H]4O)cc3nc2N1] | 1-21 |
| 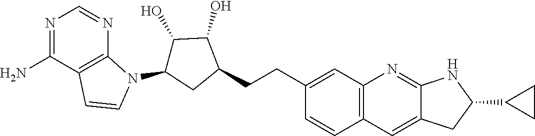 [SMILES: Nc1ncnc2c1ccn2[C@@H]1C[C@H](CCc2ccc3cc4c(nc3c2)N[C@H](C2CC2)C4)[C@@H](O)[C@H]1O] | 1-22 |
| 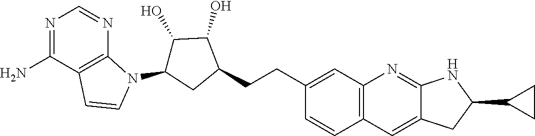 [SMILES: Nc1ncnc2c1ccn2[C@@H]1C[C@H](CCc2ccc3cc4c(nc3c2)N[C@@H](C2CC2)C4)[C@@H](O)[C@H]1O] | 1-23 |
| 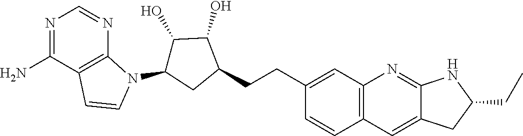 [SMILES: CC[C@@H]1Cc2cc3ccc(CC[C@H]4C[C@@H](n5ccc6c(N)ncnc65)[C@H](O)[C@@H]4O)cc3nc2N1] | 1-24 |
| 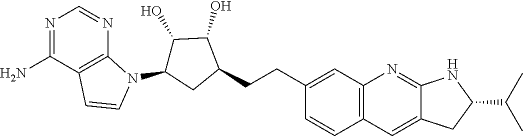 [SMILES: CC(C)[C@@H]1Cc2cc3ccc(CC[C@H]4C[C@@H](n5ccc6c(N)ncnc65)[C@H](O)[C@@H]4O)cc3nc2N1] | 1-25 |
| 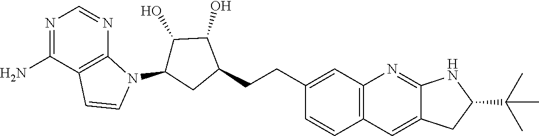 [SMILES: CC(C)(C)[C@@H]1Cc2cc3ccc(CC[C@H]4C[C@@H](n5ccc6c(N)ncnc65)[C@H](O)[C@@H]4O)cc3nc2N1] | 1-26 |
| 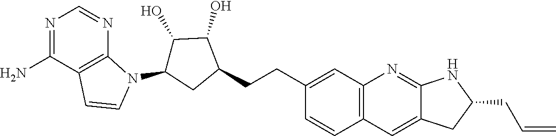 [SMILES: C=CC[C@@H]1Cc2cc3ccc(CC[C@H]4C[C@@H](n5ccc6c(N)ncnc65)[C@H](O)[C@@H]4O)cc3nc2N1] | 1-27 |
| 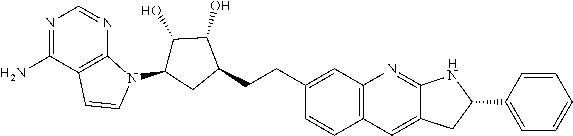 [SMILES: Nc1ncnc2c1ccn2[C@@H]1C[C@H](CCc2ccc3cc4c(nc3c2)N[C@H](c2ccccc2)C4)[C@@H](O)[C@H]1O] | 1-28 |
| 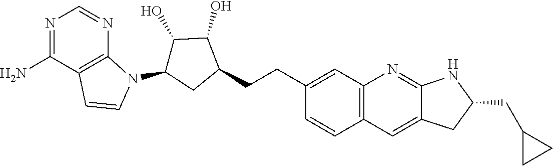 [SMILES: Nc1ncnc2c1ccn2[C@@H]1C[C@H](CCc2ccc3cc4c(nc3c2)N[C@H](CC2CC2)C4)[C@@H](O)[C@H]1O] | 1-29 |
| 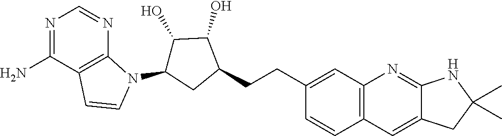 [SMILES: CC1(C)Cc2cc3ccc(CC[C@H]4C[C@@H](n5ccc6c(N)ncnc65)[C@H](O)[C@@H]4O)cc3nc2N1] | 1-30 |
| 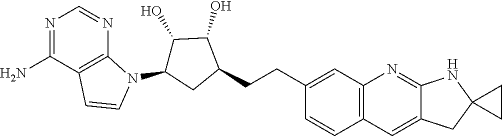 [SMILES: Nc1ncnc2c1ccn2[C@@H]1C[C@H](CCc2ccc3cc4c(nc3c2)NC2(CC2)C4)[C@@H](O)[C@H]1O] | 1-31 |
| 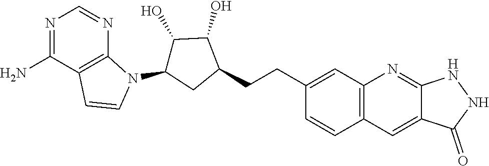 [SMILES: Nc1ncnc2c1ccn2[C@@H]1C[C@H](CCc2ccc3cc4c(=O)[nH][nH]c4nc3c2)[C@@H](O)[C@H]1O] | 1-32 |
| 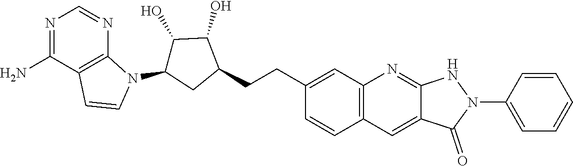 [SMILES: Nc1ncnc2c1ccn2[C@@H]1C[C@H](CCc2ccc3cc4c(=O)n(-c5ccccc5)[nH]c4nc3c2)[C@@H](O)[C@H]1O] | 1-33 |
| 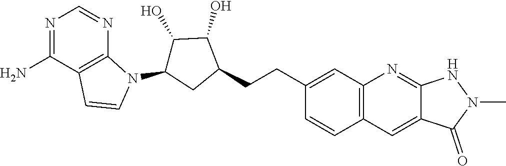 [SMILES: Cn1[nH]c2nc3cc(CC[C@H]4C[C@@H](n5ccc6c(N)ncnc65)[C@H](O)[C@@H]4O)ccc3cc2c1=O] | 1-34 |
| 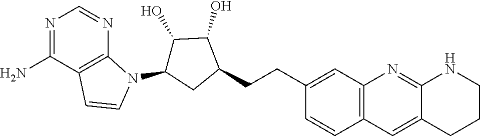 [SMILES: Nc1ncnc2c1ccn2[C@@H]1C[C@H](CCc2ccc3cc4c(nc3c2)NCCC4)[C@@H](O)[C@H]1O] | 1-35 |
| 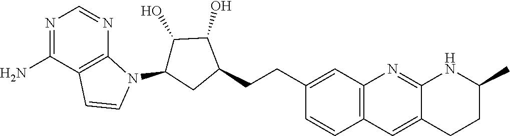 [SMILES: C[C@H]1CCc2cc3ccc(CC[C@H]4C[C@@H](n5ccc6c(N)ncnc65)[C@H](O)[C@@H]4O)cc3nc2N1] | 1-36 |
| 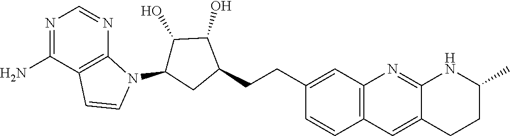 [SMILES: C[C@@H]1CCc2cc3ccc(CC[C@H]4C[C@@H](n5ccc6c(N)ncnc65)[C@H](O)[C@@H]4O)cc3nc2N1] | 1-37 |
| 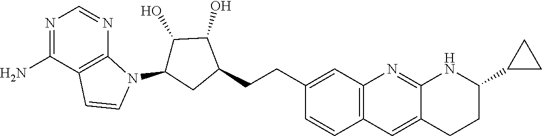 [SMILES: Nc1ncnc2c1ccn2[C@@H]1C[C@H](CCc2ccc3cc4c(nc3c2)N[C@H](C2CC2)CC4)[C@@H](O)[C@H]1O] | 1-38 |
| 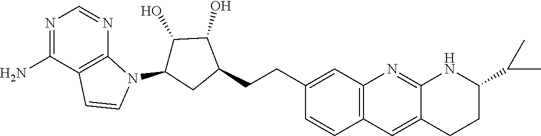 [SMILES: CC(C)[C@@H]1CCc2cc3ccc(CC[C@H]4C[C@@H](n5ccc6c(N)ncnc65)[C@H](O)[C@@H]4O)cc3nc2N1] | 1-39 |
| 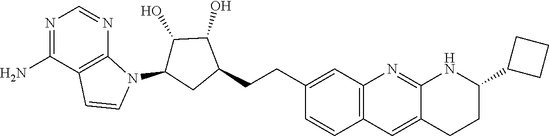 [SMILES: Nc1ncnc2c1ccn2[C@@H]1C[C@H](CCc2ccc3cc4c(nc3c2)N[C@H](C2CCC2)CC4)[C@@H](O)[C@H]1O] | 1-40 |
| 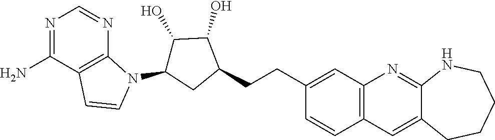 [SMILES: Nc1ncnc2c1ccn2[C@@H]1C[C@H](CCc2ccc3cc4c(nc3c2)NCCCC4)[C@@H](O)[C@H]1O] | 1-41 |
| 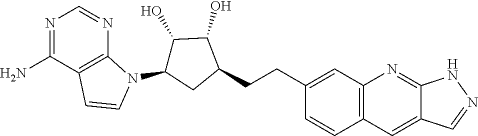 [SMILES: Nc1ncnc2c1ccn2[C@@H]1C[C@H](CCc2ccc3cc4cn[nH]c4nc3c2)[C@@H](O)[C@H]1O] | 3-12 |
| 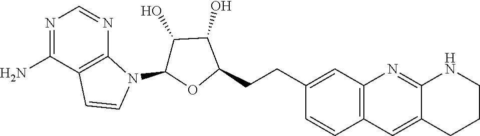 [SMILES: Nc1ncnc2c1ccn2[C@@H]1O[C@H](CCc2ccc3cc4c(nc3c2)NCCC4)[C@@H](O)[C@H]1O] | 3-13 |
| 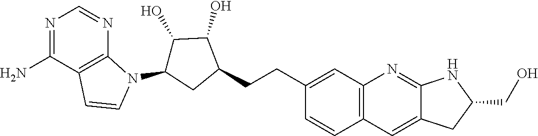 [SMILES: Nc1ncnc2c1ccn2[C@@H]1C[C@H](CCc2ccc3cc4c(nc3c2)N[C@H](CO)C4)[C@@H](O)[C@H]1O] | 1-42 |
| 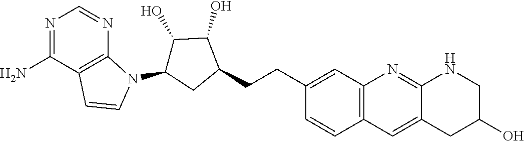 [SMILES: Nc1ncnc2c1ccn2[C@@H]1C[C@H](CCc2ccc3cc4c(nc3c2)NCC(O)C4)[C@@H](O)[C@H]1O] | 1-43 |

Some embodiments include one of the compounds listed in Table 1a below, wherein each structure can be optionally substituted.

TABLE 1a
Compound structures and their identification numbers
Compound Structure and ID Number

| | |
|---|---|
| 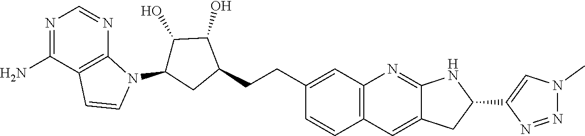 [SMILES: Cn1cc([C@@H]2Cc3cc4ccc(CC[C@H]5C[C@@H](n6ccc7c(N)ncnc76)[C@H](O)[C@@H]5O)cc4nc3N2)nn1] | 1-aa |
| 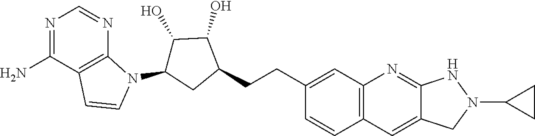 [SMILES: Nc1ncnc2c1ccn2[C@@H]1C[C@H](CCc2ccc3cc4c(nc3c2)NN(C2CC2)C4)[C@@H](O)[C@H]1O] | 1-aaa |
| 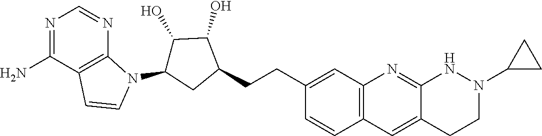 [SMILES: Nc1ncnc2c1ccn2[C@@H]1C[C@H](CCc2ccc3cc4c(nc3c2)NN(C2CC2)CC4)[C@@H](O)[C@H]1O] | 1-aaaa |

Some embodiments include use of a subject compound in the manufacture of a medicament for the treatment of cancer, infectious diseases, and other PRMT5 related disorders.

A pharmaceutical composition comprising a subject compound may be adapted for oral, or parental, such as intravenous, intramuscular, topical, intraperitoneal, nasal, buccal, sublingual, or subcutaneous administration, or for administration via respiratory tract in the form of, for example, an aerosol or an air-suspended fine powder. The dosage of a subject compound may vary depending on the route of administration, body weight, age, the type and condition of the disease being treated. A pharmaceutical composition provided herein may optionally comprise two or more subject compounds without an additional therapeutic agent, or may comprise an additional therapeutic agent (i.e., a therapeutic agent other than a compound provided herein). For example, the compounds of the disclosure can be used in combination with at least one other therapeutic agent. Therapeutic agents include, but are not limited to antibiotics, antiemetic agents, antidepressants, and antifungal agents, anti-inflammatory agents, antiviral agents, and anticancer agents that are known in the art. The pharmaceutical composition may be used for the treatment of cancer, and other PRMT5-related diseases or disorders in patients. The term “patient” herein means a mammal (e.g., a human or an animal). In some embodiments, the patient has cancer.

The pharmaceutical composition described herein can be prepared by combining a compound of Formula 1 with at least one pharmaceutical acceptable inert ingredient, such as a carrier, excipient, filler, lubricant, flavoring agent, buffer, etc., selected on the basis of the chosen route of administration and standard pharmaceutical practice as described, for example, in Remington's Pharmaceutical Sciences, 2005, the disclosure of which is hereby incorporated herein by reference, in its entirety. The relative proportions of active ingredient and carrier may be determined, for example, by the solubility and chemical nature of the compounds, chosen route of administration and standard pharmaceutical practice.

Some embodiments include a method of treating a disease or disorder associated with PRMT5 comprising administering a therapeutically effective amount of a compound of Formula 1 or a pharmaceutical composition comprising a compound of Formula 1 to a patient in need thereof. The term a “therapeutically effective amount” herein refers to an amount of a compound or a pharmaceutical composition of the present disclosure provided herein sufficient to be effective in inhibiting PRMT5 enzyme and thus providing a benefit in the treatment of cancer, infectious diseases and other PRMT5 associated disorders, to delay or minimize symptoms associated with cancer, infectious diseases and other PRMT5 associated disorders, or to ameliorate a disease or infection or cause thereof (e.g. 0.1-1000 mg). The term “treatment” refers to causing a therapeutically beneficial effect, such as ameliorating existing symptoms, ameliorating the underlying causes of symptoms, postponing, preventing the further development of a disorder, or reducing the severity of symptoms that are otherwise expected to develop without treatment.

Some embodiments include a kit containing an effective therapeutic amount of a subject compound and a label with instructions to use the subject compound, or a composition or dosage form containing an effective therapeutic amount of the subject compound, for the treatment of cancer, infectious diseases, and/or other PRMT5 related disorders.

The following embodiments are contemplated:

### Embodiment 1

A compound represented by a formula:

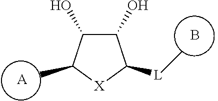

or a pharmaceutically acceptable salt thereof;
wherein

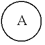

(Ring A) is an optionally substituted 4-amino-7H-pyrrolo[2,3-d]pyrimidin-7-yl;

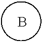

(Ring B) is an optionally substituted fused tricyclic heterocyclic ring system containing 1, 2, 3, 4, or 5 ring N atoms, 0 or 1 ring O atom, and a fused benzene ring, wherein the fused benzene ring is directly attached to L;
X is —O—, —CH₂—, or —CF₂—;
L is optionally substituted C₁₋₃ hydrocarbylene, optionally substituted —O—C₁₋₂ hydrocarbylene-, optionally substituted —S—C₁₋₂ hydrocarbylene-, or optionally substituted —NR^{A}—C₁₋₂ hydrocarbylene-; and
R^{A} is H, C₁₋₆ hydrocarbyl, C₁₋₆ heteroaryl, C₁₋₆ heterocycloalkyl, —C(O)—C₁₋₆ alkyl, —C(O)NH—C₁₋₆ alkyl, or —C(O)OC₁₋₆ alkyl.

### Embodiment 2

The compound of embodiment 1, wherein Ring A comprises:

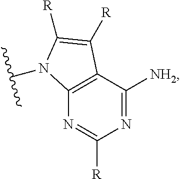

and Ring B comprises:

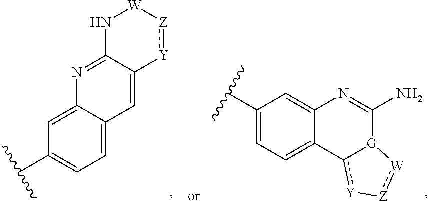

wherein each structure is optionally substituted;

### G is N or CR;

Y is —N(R^{A})—, N, C(R^{C}), or —C(R^{C}R^{D})—, or —C(R^{C}R^{D})—C(R^{C}R^{D})—;
Z is a bond, —N(R^{A})—, N, C(R^{C}), or —C(R^{C}R^{D})—;
W is a bond, —N(R^{A})—, N, —O—, C(R^{C}), or —C(R^{C}R^{D})—;
dashed line represents optionally with or without a bond;
each R is independently H, F, Cl, Br, I, —NR^{A}R^{B}, C₁₋₆ hydrocarbyl, —OH, —CN, or —O—C₁₋₆ alkyl;
each R^{C}, and each R^{D} are independently H, F, Cl, Br, I, —NR^{A}R^{B}, C₁₋₆ hydrocarbyl, —OH, —CN, ═O, or —O—C₁₋₆ alkyl;
each R^{A} and each R^{B} are independently H, C₁₋₆ hydrocarbyl, C₁₋₆ heteroaryl, C₁₋₆ heterocycloalkyl, —C(O)—C₁₋₆ alkyl, —C(O)NH—C₁₋₆ alkyl, or —C(O)OC₁₋₆ alkyl; and
wherein each R, each R^{A}, each R^{B}, each R^{C}, and each R^{D} are independently optionally halogenated.

### Embodiment 3

The compound of embodiment 1 or 2, wherein Ring A comprises unsubstituted 4-amino-7H-pyrrolo[2,3-d]pyrimidin-7-yl.

### Embodiment 4

The compound of embodiment 1, 2, or 3, wherein the optionally substituted fused tricyclic heterocyclic ring system of Ring B is an optionally substituted fused tricyclic heteroaromatic ring system.

### Embodiment 5

The compound of embodiment 1, 2, or 3, wherein Ring B contains one ring N atom.

### Embodiment 6

The compound of embodiment 1, 2, or 3, wherein Ring B contains two ring N atoms.

### Embodiment 7

The compound of embodiment 1, 2, or 3, wherein Ring B contains three ring N atoms.

### Embodiment 8

The compound of embodiment 1, 2, or 3, wherein Ring B contains four ring N atoms.

### Embodiment 9

The compound of embodiment 1, 2, or 3, wherein Ring B contains one ring O atom.

### Embodiment 10

The compound of embodiment 1, 2, or 3, wherein Ring B is optionally substituted 2,3-dihydro-1H-pyrrolo[2,3-b]quinolin-7-yl, optionally substituted 3,3-dimethyl-2,3-dihydro-1H-pyrrolo[2,3-b]quinolin-7-yl, optionally substituted 1H-pyrrolo[2,3-b]quinolin-7-yl, optionally substituted 1H-pyrazolo[3,4-b]quinolin-7-yl, optionally substituted 5-amino-2,3-dihydroimidazo[1,2-c]quinazolin-8-yl, optionally substituted 5-aminoimidazo[1,2-c]quinazolin-8-yl, optionally substituted (1aS,7bR)-2-amino-1a,7b-dihydro-1H-cyclopropa[c]quinolin-5-yl, optionally substituted (1aR,7bS)-2-amino-1a,7b-dihydro-1H-cyclopropa[c]quinolin-5-yl, optionally substituted 3,4-dihydro-1H-[1,2]oxazino[3,4-b]quinolin-8-yl, optionally substituted 1,3-dihydroisoxazolo[3,4-b]quinolin-7-yl, optionally substituted (R)-3-methyl-3,4-dihydro-1H-[1,2]oxazino[3,4-b]quinolin-8-yl, optionally substituted 3H-imidazo[4,5-b]quinolin-6-yl, optionally substituted (S)-2-methyl-2,3-dihydro-1H-pyrrolo[2,3-b]quinolin-7-yl, optionally substituted (R)-2-methyl-2,3-dihydro-1H-pyrrolo[2,3-b]quinolin-7-yl, optionally substituted (S)-2-cyclopropyl-2,3-dihydro-1H-pyrrolo[2,3-b]quinolin-7-yl, optionally substituted (R)-2-cyclopropyl-2,3-dihydro-1H-pyrrolo[2,3-b]quinolin-7-yl, optionally substituted (R)-2-ethyl-2,3-dihydro-1H-pyrrolo[2,3-b]quinolin-7-yl, optionally substituted (S)-2-isopropyl-2,3-dihydro-1H-pyrrolo[2,3-b]quinolin-7-yl, optionally substituted (S)-2-(tert-butyl)-2,3-dihydro-1H-pyrrolo[2,3-b]quinolin-7-yl, optionally substituted (R)-2-allyl-2,3-dihydro-1H-pyrrolo[2,3-b]quinolin-7-yl, optionally substituted 2,2-dimethyl-2,3-dihydro-1H-pyrrolo[2,3-b]quinolin-7-yl, optionally substituted 1′,3′-dihydrospiro[cyclopropane-1,2′-pyrrolo[2,3-b]quinolin]-7′-yl, optionally substituted 1,2,3,4-tetrahydrobenzo[b][1,8]naphthyridin-8-yl, optionally substituted (S)-2-methyl-1,2,3,4-tetrahydrobenzo[b][1,8]naphthyridin-8-yl, optionally substituted (R)-2-methyl-1,2,3,4-tetrahydrobenzo[b][1,8]naphthyridin-8-yl, optionally substituted (S)-2-cyclopropyl-1,2,3,4-tetrahydrobenzo[b][1,8]naphthyridin-8-yl, optionally substituted 2,3,4,5-tetrahydro-1H-azepino[2,3-b]quinolin-9-yl, optionally substituted (S)-(2,3-dihydro-1H-pyrrolo[2,3-b]quinolin-2-yl)methanol, optionally substituted 2-cyclopropyl-2,3-dihydro-1H-pyrazolo[3,4-b]quinolin-7-yl, or optionally substituted 1,2,3,4-tetrahydrobenzo[b][1,8]naphthyridin-3-ol.

### Embodiment 11

The compound of embodiment 1, 2, or 3, wherein Ring B comprises optionally substituted 2,3-dihydro-1H-pyrrolo[2,3-b]quinolin-7-yl.

### Embodiment 12

The compound of embodiment 1, 2, or 3, wherein Ring B comprises optionally substituted 3,3-dimethyl-2,3-dihydro-1H-pyrrolo[2,3-b]quinolin-7-yl.

### Embodiment 13

The compound of embodiment 1, 2, or 3, wherein Ring B comprises optionally substituted 1H-pyrrolo[2,3-b]quinolin-7-yl.

### Embodiment 14

The compound of embodiment 1, 2, or 3, wherein Ring B comprises optionally substituted 1H-pyrazolo[3,4-b]quinolin-7-yl.

### Embodiment 15

The compound of embodiment 1, 2, or 3, wherein Ring B comprises optionally substituted 5-amino-2,3-dihydroimidazo[1,2-c]quinazolin-8-yl.

### Embodiment 16

The compound of embodiment 1, 2, or 3, wherein Ring B comprises optionally substituted 5-aminoimidazo[1,2-c]quinazolin-8-yl.

### Embodiment 17

The compound of embodiment 1, 2, or 3, wherein Ring B comprises optionally substituted (1aS,7bR)-2-amino-1a,7b-dihydro-1H-cyclopropa[c]quinolin-5-yl.

### Embodiment 18

The compound of embodiment 1, 2, or 3, wherein Ring B comprises optionally substituted (1aR,7bS)-2-amino-1a,7b-dihydro-1H-cyclopropa[c]quinolin-5-yl.

### Embodiment 19

The compound of embodiment 1, 2, or 3, wherein Ring B comprises optionally substituted 3,4-dihydro-1H-[1,2]oxazino[3,4-b]quinolin-8-yl.

### Embodiment 20

The compound of embodiment 1, 2, or 3, wherein Ring B comprises optionally substituted 1,3-dihydroisoxazolo[3,4-b]quinolin-7-yl.

### Embodiment 21

The compound of embodiment 1, 2, or 3, wherein Ring B comprises optionally substituted (R)-3-methyl-3,4-dihydro-1H-[1,2]oxazino[3,4-b]quinolin-8-yl Embodiment 22. The compound of embodiment 1, 2, or 3, wherein Ring B comprises optionally substituted 3H-imidazo[4,5-b]quinolin-6-yl.

### Embodiment 23

The compound of embodiment 1, 2, or 3, wherein Ring B comprises optionally substituted (S)-2-methyl-2,3-dihydro-1H-pyrrolo[2,3-b]quinolin-7-yl.

### Embodiment 24

The compound of embodiment 1, 2, or 3, wherein Ring B comprises optionally substituted (R)-2-methyl-2,3-dihydro-1H-pyrrolo[2,3-b]quinolin-7-yl.

### Embodiment 25

The compound of embodiment 1, 2, or 3, wherein Ring B comprises optionally substituted (S)-2-cyclopropyl-2,3-dihydro-1H-pyrrolo[2,3-b]quinolin-7-yl.

### Embodiment 26

The compound of embodiment 1, 2, or 3, wherein Ring B comprises optionally substituted (R)-2-cyclopropyl-2,3-dihydro-1H-pyrrolo[2,3-b]quinolin-7-yl.

### Embodiment 27

The compound of embodiment 1, 2, or 3, wherein Ring B comprises optionally substituted (R)-2-ethyl-2,3-dihydro-1H-pyrrolo[2,3-b]quinolin-7-yl.

### Embodiment 28

The compound of embodiment 1, 2, or 3, wherein Ring B comprises optionally substituted (S)-2-isopropyl-2,3-dihydro-1H-pyrrolo[2,3-b]quinolin-7-yl.

### Embodiment 29

The compound of embodiment 1, 2, or 3, wherein Ring B comprises optionally substituted (S)-2-(tert-butyl)-2,3-dihydro-1H-pyrrolo[2,3-b]quinolin-7-yl.

### Embodiment 30

The compound of embodiment 1, 2, or 3, wherein Ring B comprises optionally substituted (R)-2-allyl-2,3-dihydro-1H-pyrrolo[2,3-b]quinolin-7-yl.

### Embodiment 31

The compound of embodiment 1, 2, or 3, wherein Ring B comprises optionally substituted 2,2-dimethyl-2,3-dihydro-1H-pyrrolo[2,3-b]quinolin-7-yl.

### Embodiment 32

The compound of embodiment 1, 2, or 3, wherein Ring B comprises optionally substituted 1′,3′-dihydrospiro[cyclopropane-1,2′-pyrrolo[2,3-b]quinolin]-7′-yl.

### Embodiment 33

The compound of embodiment 1, 2, or 3, wherein Ring B comprises optionally substituted 1,2,3,4-tetrahydrobenzo[b][1,8]naphthyridin-8-yl.

### Embodiment 34

The compound of embodiment 1, 2, or 3, wherein Ring B comprises optionally substituted (S)-2-methyl-1,2,3,4-tetrahydrobenzo[b][1,8]naphthyridin-8-yl.

### Embodiment 35

The compound of embodiment 1, 2, or 3, wherein Ring B comprises optionally substituted (R)-2-methyl-1,2,3,4-tetrahydrobenzo[b][1,8]naphthyridin-8-yl.

### Embodiment 36

The compound of embodiment 1, 2, or 3, wherein Ring B comprises optionally substituted (S)-2-cyclopropyl-1,2,3,4-tetrahydrobenzo[b][1,8]naphthyridin-8-yl.

### Embodiment 37

The compound of embodiment 1, 2, or 3, wherein Ring B comprises optionally substituted 2,3,4,5-tetrahydro-1H-azepino[2,3-b]quinolin-9-yl.

### Embodiment 38

The compound of embodiment 1, 2, 3, 4, 5, 6, 7, 8, 9, 10, 11, 12, or 13, 14, 15, 16, 17, 18, 19, 20, 21, 22, 23, 24, 25, 26, 27, 28, 29, 30, 31, 32, 33, 34, 35, 36, or 37, wherein X is —CH₂—.

### Embodiment 39

The compound of embodiment 1, 2, 3, 4, 5, 6, 7, 8, 9, 10, 11, 12, or 13, 14, 15, 16, 17, 18, 19, 20, 21, 22, 23, 24, 25, 26, 27, 28, 29, 30, 31, 32, 33, 34, 35, 36, or 37, wherein X is —O—.

### Embodiment 40

The compound of embodiment 1, 2, 3, 4, 5, 6, 7, 8, 9, 10, 11, 12, or 13, 14, 15, 16, 17, 18, 19, 20, 21, 22, 23, 24, 25, 26, 27, 28, 29, 30, 31, 32, 33, 34, 35, 36, or 37, wherein X is —CF₂—.

### Embodiment 41

The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, wherein L is —CH₂—CH₂—.

### Embodiment 42

The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, wherein L is —CH₂—CH₂—CH₂-Embodiment 43. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, wherein L is —CH₂O—.

### Embodiment 44

The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, wherein L is —O—CH₂—.

### Embodiment 45

A compound, or a pharmaceutically acceptable salt thereof, wherein the compound is optionally substituted (1S,2R,3S,5R)-3-(2-(2,3-dihydro-1H-pyrrolo[2,3-b]quinolin-7-yl)ethyl)-5-(7H-pyrrolo[2,3-d]pyrimidin-7-yl)cyclopentane-1,2-diol, optionally substituted (1S,2R,3S,5R)-3-(2-(2,3-dihydroimidazo[1,2-c]quinazolin-8-yl)ethyl)-5-(7H-pyrrolo[2,3-d]pyrimidin-7-yl)cyclopentane-1,2-diol, optionally substituted (1S,2R,3S,5R)-3-(2-(imidazo[1,2-c]quinazolin-8-yl)ethyl)-5-(7H-pyrrolo[2,3-d]pyrimidin-7-yl)cyclopentane-1,2-diol, optionally substituted (1S,2R,3S,5R)-3-(2-((1aS,7bR)-1a,7b-dihydro-1H-cyclopropa[c]quinolin-5-yl)ethyl)-5-(7H-pyrrolo[2,3-d]pyrimidin-7-yl)cyclopentane-1,2-diol, optionally substituted (1S,2R,3S,5R)-3-(2-((1aR,7bS)-1a,7b-dihydro-1H-cyclopropa[c]quinolin-5-yl)ethyl)-5-(7H-pyrrolo[2,3-d]pyrimidin-7-yl)cyclopentane-1,2-diol, optionally substituted (1S,2R,3S,5R)-3-(2-(1H-pyrazolo[3,4-b]quinolin-7-yl)ethyl)-5-(7H-pyrrolo[2,3-d]pyrimidin-7-yl)cyclopentane-1,2-diol, optionally substituted (2R,3S,4R,5R)-2-(2-(2,3-dihydro-1H-pyrrolo[2,3-b]quinolin-7-yl)ethyl)-5-(7H-pyrrolo[2,3-d]pyrimidin-7-yl)tetrahydrofuran-3,4-diol, optionally substituted (2R,3S,4R,5R)-2-(2-(2,3-dihydroimidazo[1,2-c]quinazolin-8-yl)ethyl)-5-(7H-pyrrolo[2,3-d]pyrimidin-7-yl)tetrahydrofuran-3,4-diol, optionally substituted (2R,3S,4R,5R)-2-(2-(imidazo[1,2-c]quinazolin-8-yl)ethyl)-5-(7H-pyrrolo[2,3-d]pyrimidin-7-yl)tetrahydrofuran-3,4-diol, optionally substituted (1S,2R,3S,5R)-3-(2-(1H-pyrrolo[2,3-b]quinolin-7-yl)ethyl)-5-(7H-pyrrolo[2,3-d]pyrimidin-7-yl)cyclopentane-1,2-diol, (1S,2R,3S,5R)-3-(2-(3,4-dihydro-1H-[1,2]oxazino[3,4-b]quinolin-8-yl)ethyl)-5-(7H-pyrrolo[2,3-d]pyrimidin-7-yl)cyclopentane-1,2-diol, (1S,2R,3S,5R)-3-(2-(1,3-dihydroisoxazolo[3,4-b]quinolin-7-yl)ethyl)-5-(7H-pyrrolo[2,3-d]pyrimidin-7-yl)cyclopentane-1,2-diol, optionally substituted (1R,2S,3R,5S)-3-(7H-pyrrolo[2,3-d]pyrimidin-7-yl)-5-(2-(1,3,4,5-tetrahydro-[1,2]oxazepino[3,4-b]quinolin-9-yl)ethyl)cyclopentane-1,2-diol, optionally substituted (1S,2R,3S,5R)-3-(2-(3H-imidazo[4,5-b]quinolin-6-yl)ethyl)-5-(7H-pyrrolo[2,3-d]pyrimidin-7-yl)cyclopentane-1,2-diol, optionally substituted (1S,2R,3S,5R)-3-(2-(3H-[1,2,3]triazolo[4,5-b]quinolin-6-yl)ethyl)-5-(7H-pyrrolo[2,3-d]pyrimidin-7-yl)cyclopentane-1,2-diol, optionally substituted (1S,2R,3S,5R)-3-(2-(2,3-dihydro-1H-pyrazolo[3,4-b]quinolin-7-yl)ethyl)-5-(7H-pyrrolo[2,3-d]pyrimidin-7-yl)cyclopentane-1,2-diol, optionally substituted (1R,2S,3R,5S)-3-(7H-pyrrolo[2,3-d]pyrimidin-7-yl)-5-(2-(1,2,3,4-tetrahydrobenzo[b][1,8]naphthyridin-8-yl)ethyl)cyclopentane-1,2-diol, optionally substituted (1R,2S,3R,5S)-3-(7H-pyrrolo[2,3-d]pyrimidin-7-yl)-5-(2-(2,3,4,5-tetrahydro-1H-azepino[2,3-b]quinolin-9-yl)ethyl)cyclopentane-1,2-diol, optionally substituted (2R,3S,4R,5R)-2-(2-(1H-pyrazolo[3,4-b]quinolin-7-yl)ethyl)-5-(7H-pyrrolo[2,3-d]pyrimidin-7-yl)tetrahydrofuran-3,4-diol, optionally substituted (2R,3R,4S,5R)-2-(7H-pyrrolo[2,3-d]pyrimidin-7-yl)-5-(2-(1,2,3,4-tetrahydrobenzo[b][1,8]naphthyridin-8-yl)ethyl)tetrahydrofuran-3,4-diol, or optionally substituted (1R,2S,3R,5S)-3-(7H-pyrrolo[2,3-d]pyrimidin-7-yl)-5-(2-(1,2,3,4-tetrahydropyridazino[3,4-b]quinolin-8-yl)ethyl)cyclopentane-1,2-diol.

### Embodiment 46

The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein the compound is an R-enantiomer.

### Embodiment 47

The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein the compound is an S-enantiomer.

### Embodiment 48

The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or 47, wherein the compound is deuterated.

### Embodiment 49

The compound of embodiment 1, 2, 3, 4, 5, 6, 7, 8, 9, 10, 11, 12, 13, 14, 15, 16, 17, 18, 19, 20, 21, 22, 23, 24, 25, 26, 27, or 28, wherein each substituent, if present, of Ring A, Ring B, and L, has a molecular weight of 15 mg/mL to 200 mg/mL.

### Embodiment 50

A compound, or a pharmaceutically acceptable salt thereof, wherein the compound is:

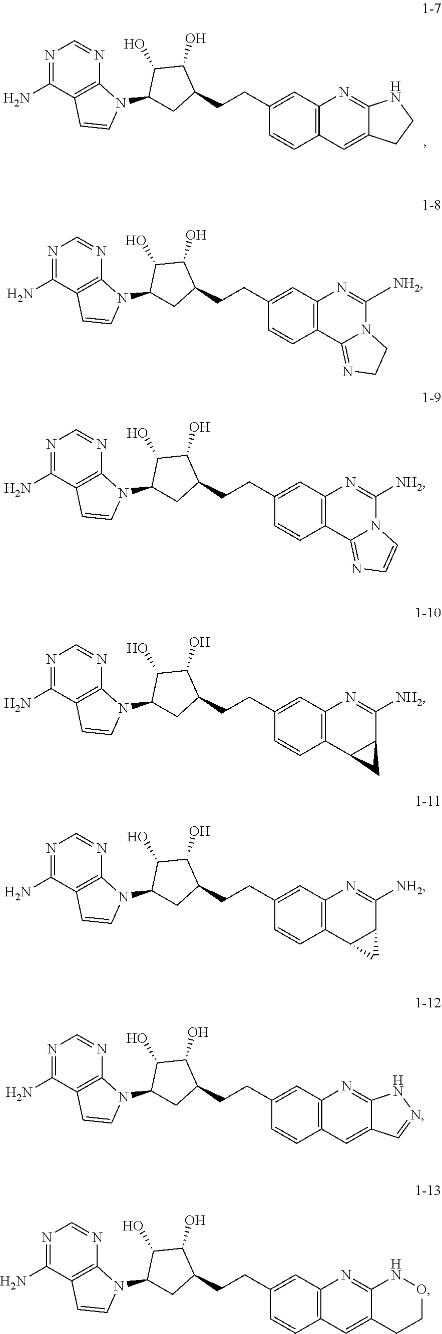

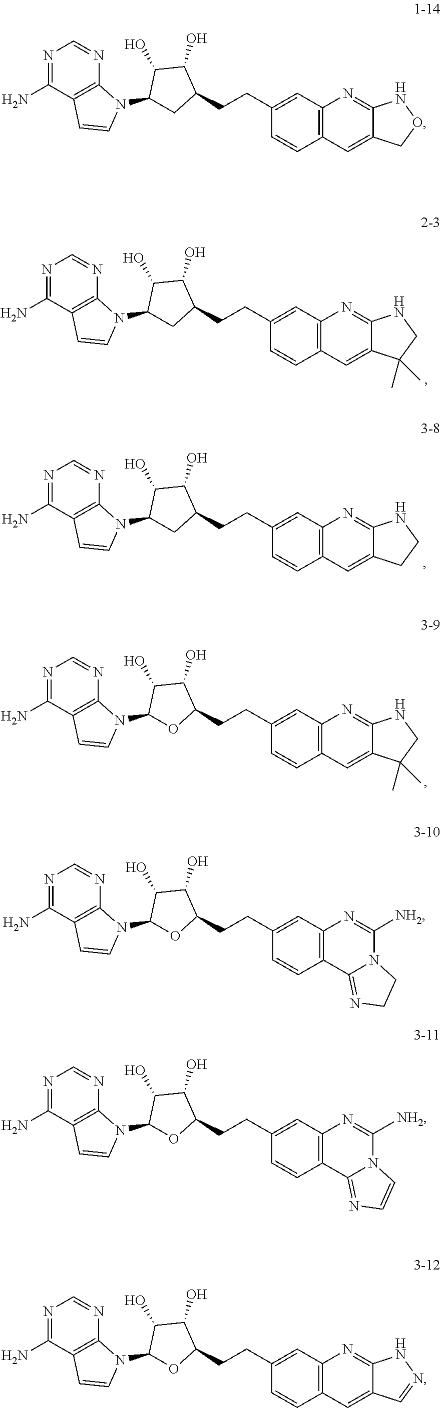

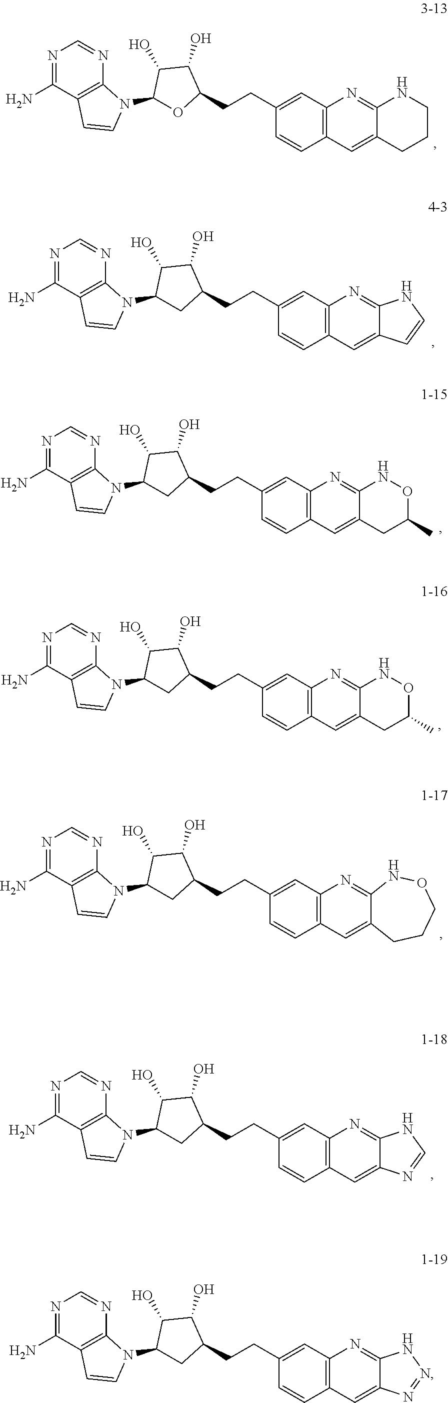

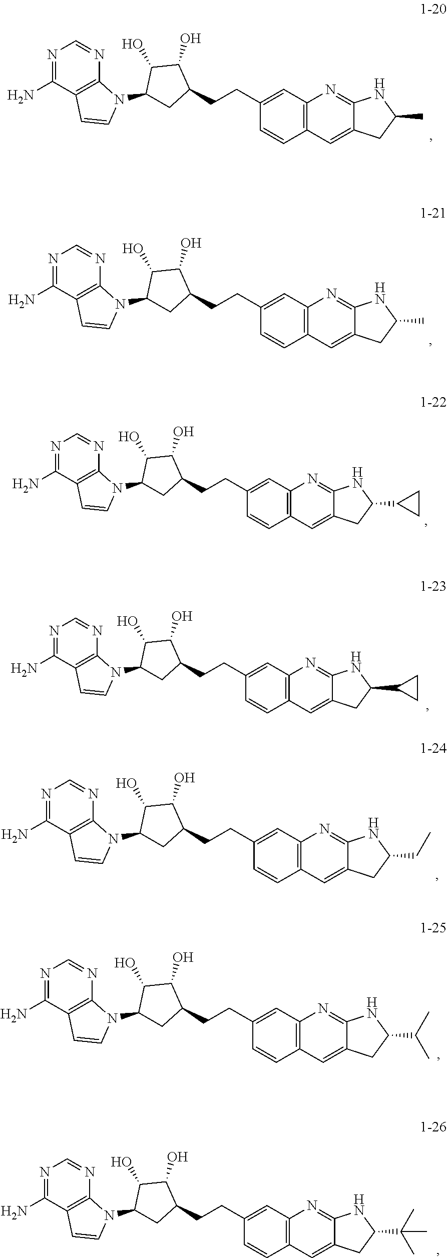

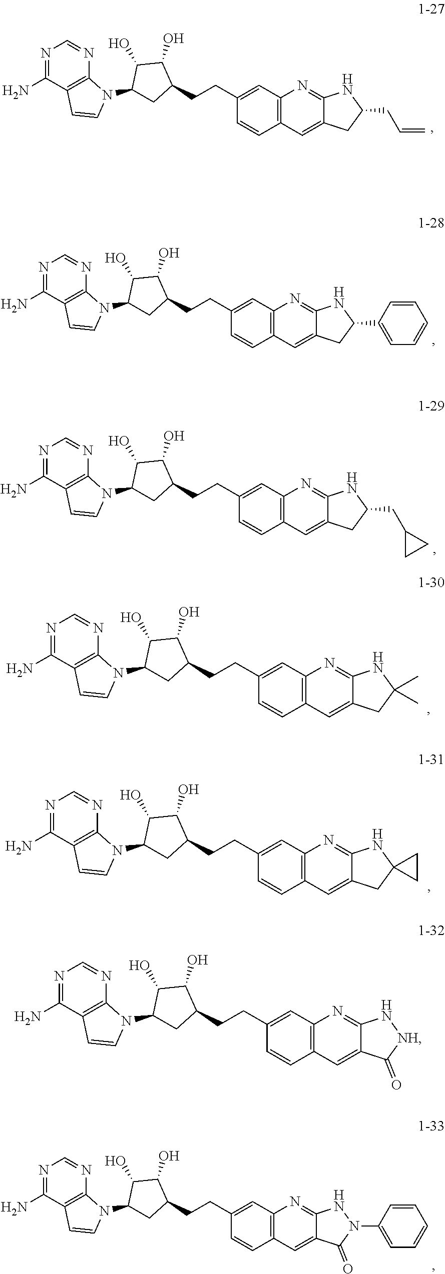

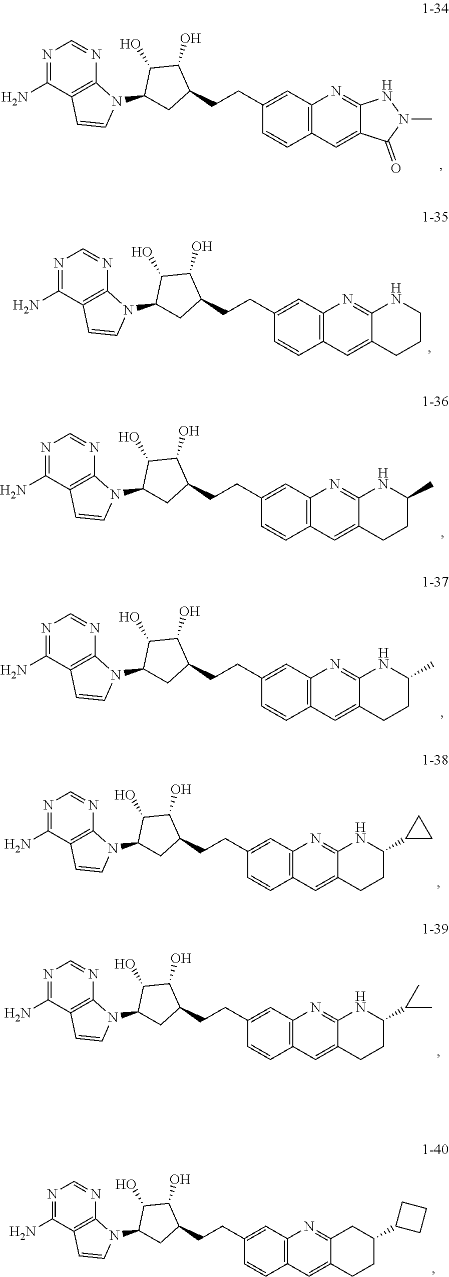

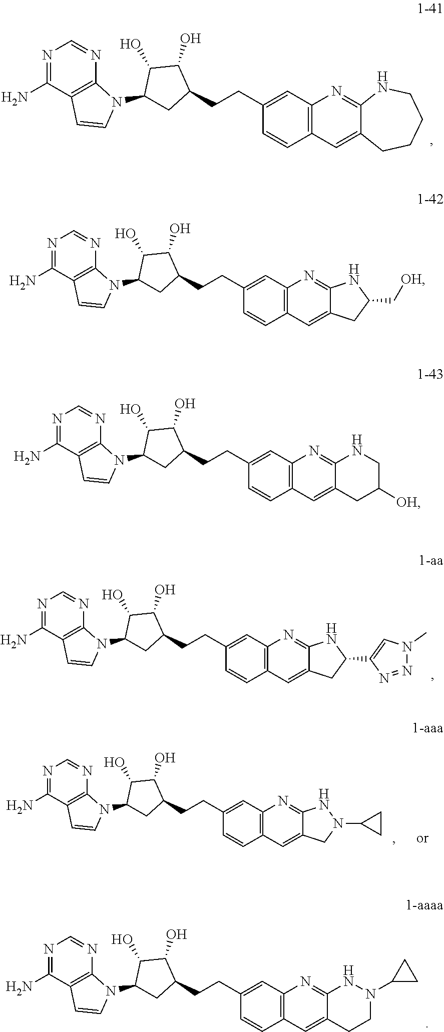 [SMILES: Nc1ncnc2c1ccn2[C@@H]1C[C@H](CCc2ccc3cc4c(nc3c2)NCCCC4)[C@@H](O)[C@H]1O ; Nc1ncnc2c1ccn2[C@@H]1C[C@H](CCc2ccc3cc4c(nc3c2)N[C@H](CO)C4)[C@@H](O)[C@H]1O ; Nc1ncnc2c1ccn2[C@@H]1C[C@H](CCc2ccc3cc4c(nc3c2)NCC(O)C4)[C@@H](O)[C@H]1O ; Cn1cc([C@@H]2Cc3cc4ccc(CC[C@H]5C[C@@H](n6ccc7c(N)ncnc76)[C@H](O)[C@@H]5O)cc4nc3N2)nn1 ; Nc1ncnc2c1ccn2[C@@H]1C[C@H](CCc2ccc3cc4c(nc3c2)NN(C2CC2)C4)[C@@H](O)[C@H]1O ; Nc1ncnc2c1ccn2[C@@H]1C[C@H](CCc2ccc3cc4c(nc3c2)NN(C2CC2)CC4)[C@@H](O)[C@H]1O]

### Embodiment 51

A method of treating cancer, infectious diseases, and other PRMT5-related diseases or disorders comprising administering a compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or a pharmaceutically acceptable salt thereof, to a patient in need thereof.

### Embodiment 52

Use of a compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or a pharmaceutically acceptable salt thereof, in the manufacture of a medicament for the treatment of cancer, infectious diseases, and other PRMT5-related diseases or disorders.

### Embodiment 53

A pharmaceutical composition comprising a therapeutically effective amount of a compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or a pharmaceutically acceptable salt thereof, in combination with at least one pharmaceutically acceptable carrier.

### EXPERIMENTAL SECTION

### Preparation of Compounds

The compounds of the disclosure can be made using procedures known in the art. The following reaction schemes show typical procedures, but those skilled in the art will recognize that other procedures can also be suitable for using to prepare these compounds. For examples in Formula 1-3 and A1, wherein there is a substituent at any position of any of the structures, those skilled in the art will recognize that changes to the requisite reagents can be made at the appropriate steps in the synthetic methods outlined below. Reactions may involve monitoring for consumption of starting materials, and there are many methods for the monitoring, including but not limited to thin layer chromatography (TLC) and liquid chromatography mass spectrometry (LCMS). Those skilled in the art will recognize that any synthetic method specified in the examples shown below can be substituted by other non-limiting methods when suitable.

Some of the techniques, solvents and reagents can be referred to by their abbreviations as follows:

### Acetonitrile: MeCN or ACN

### Aqueous: aq.

### Benzyl: Bn

9-Borabicyclo [3.3.1] nonane: 9-BBN

### N,O-Bis(trimethylsilyl)acetamide: BSA

[1,1′-Bis(diphenylphosphino)ferrocene]-dichloropalladium (II): Pd(dppf)Cl₂
Cerium ammonium nitrate: CAN
meta-chloroperoxybenzoic acid: m-CPBA (or mCPBA)
1,2-dibromotetracholoroethane: DBTCE:
3-Oxo-1,3-dihydro-1λ5,2-benziodoxole-1,1,1-triyl triacetate: Dess-Martin periodinane
Hydroquinidine 1,4-phthalazinediyl diether: (DHQ)2PHAL
2,3-Dichloro-5,6-dicyano-1,4-benzoquinone: DDQ

### Dichloromethane: DCM

Diethyl azodicarboxylate: DEAD
Diisopropyl azodicarboxylate: DIAD
Diisopropylethylamine: DIPEA, DIEA or iPr₂Net

### Dimethylaminopyridine: DMAP

### Dimethylformamide: DMF

### Dimethylsulfoxide: DMSO

1-Ethyl-3-(3-dimethylaminopropyl)carbodiimide: EDCI
Equivalents: equiv.
Ether or diethyl ether: Et₂O
Ethyl acetate: AcOEt or EtOAc or EA

### Example: Ex. or ex.

Formic acid: FA

### Grams: g

High performance liquid chromatography: HPLC

### Hydroxybenzotriazole: HOBT

2-Iodoxybenzoic acid: IBX

### Inhibition: Inh.

1,3,2,4-dithiadiphosphetane, 2,4-bis(4-methoxyphenyl)-,2,4-disulfide: Lawesson's reagent
Liquid chromatography mass spectrometry: LCMS or LC-MS
Lithium aluminum hydride: LAH
Lithium hexamethyldisilazide: LiHMDS
Methansulfonyl chloride: MeSO₂Cl
Methyl iodide: Mel

### Methanol: MeOH

### Microliter: μl

### Micrometer: μm

### Milligram: mg

### Milliliter: mL

Millimole: mmol
(R)-(−)-(3,5-Dioxa-4-phosphacyclohepta[2,1-a:3,4-a′]dinaphthalen-4-yl)dimethylamine: (R)-MonoPhos
(R)-(−)-(3,5-Dioxa-4-phospha-cyclohepta[2,1-a;3,4-a′]dinaphthalen-4-yl)dimethylamine:

### (R)-MonoPhos

### N-Bromosuccinimide: NBS

n-Butyllithium: n-BuLi
Nuclear magnetic resonance spectroscopy: NMR
Palladium tetra-triphenylphosphine: Pd(PPh₃)₄
N-Phenyl bis(trifluoromethanwsulfonimide): PhNTf₂
Retentional time: t_{R}

### Acetylacetonatobis(ethylene)rhodium(I): Rh(acac)(eth)₂

Room temperature (ambient, ˜25° C.): rt or RT

### Paramethoxybenzyl: PMB

Petroleum ether: PE

### Preparative HPLC: Prep-HPLC

### Preparative TLC: Prep-TLC

Potassium tert-butoxide: t-BuOK
Sodium hydride: NaH

### Supercritical Fluid Chromatography: SFC

Tris(2-carboxymethyl)phosphine: TCEP
Temperature: temp.

### Tetrahydrofuran: THF

Thin layer chromatography: TLC
p-Toluenesulfonic acid: TsOH

### Triethylamine: Et₃N or TEA

Trifluoroacetic acid: TFA
Trifluoromethanesulfonic anhydride: Tf₂O
Trimethylsilyl trifluoro methanesulfonate: TMSOTf

In the synthetic schemes described below, unless otherwise indicated all temperatures are set forth in degrees Celsius and all parts and percentages are by weight. Reagents and solvents were purchased from commercial suppliers such as Aldrich Chemical Company and were used without further purification unless otherwise indicated. Tetrahydrofuran (THF) and N,N-dimethylformamide (DMF) were purchased from commercial sources in Sure Seal bottles and used as received.

The reactions set forth below were done generally under a positive pressure of argon or nitrogen at an ambient temperature (unless otherwise stated) in anhydrous solvents. Glassware was oven dried and/or heat dried. The reactions were assayed by TLC and/or analyzed by LC-MS and terminated as judged by the consumption of starting material. Analytical thin layer chromatography (TLC) was performed on glass plates pre-coated with silica gel 60 F254 0.25 mm plates (EM Science), and visualized with UV light (254 nm) and/or heating with commercial ethanolic phosphomolybdic acid. preparative thin layer chromatography (TLC) was performed on glass-plates pre-coated with silica gel 60 F254 0.5 mm plates (20×20 cm, from commercial sources) and visualized with UV light (254 nm).

Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ and/or Mg₂SO₄ prior to filtration and evaporation of the solvents under reduced pressure on a rotary evaporator and noted as solvents removed in vacuo. Column chromatography was completed under positive pressure using 230-400 mesh silica gel.

¹H-NMR spectra and ¹³C-NMR were recorded on a Varian Mercury-VX400 instrument operating at 400 MHZ. NMR spectra were obtained as CDCl₃ solutions (reported in ppm), using chloroform as the reference standard (7.27 ppm for the proton and 77.00 ppm for carbon), CD₃OD (3.4 and 4.8 ppm for the protons and 49.3 ppm for carbon), DMSO-d₆ (2.49 ppm for proton), or internally tetramethylsilane (0.00 ppm) when appropriate. Other NMR solvents were used as needed.

Some of the typical synthetic methods are described in the examples shown below.

### Method 1:

### Example 1: Synthesis of (1R,2S,3R,5S)-3-(4-amino-7H-pyrrolo[2,3-d]pyrimidin-7-yl)-5-(2-(2,3-dihydro-1H-pyrrolo[2,3-b]quinolin-7-yl)ethyl)cyclopentane-1,2-diol

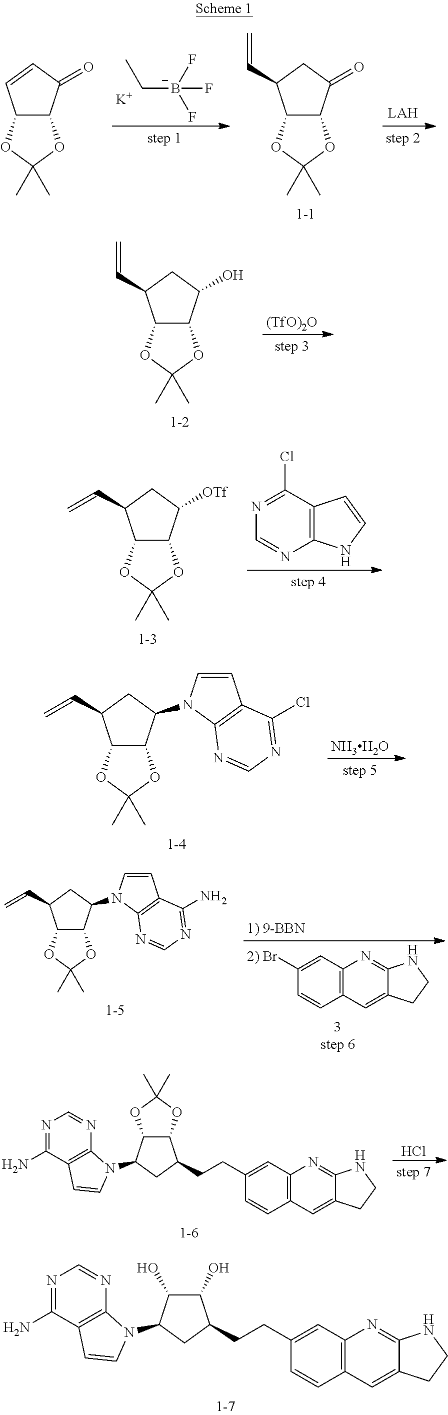

### Step 1: Synthesis of (3aR,6R,6aR)-2,2-dimethyl-6-vinyltetrahydro-4H-cyclopenta[d][1,3]dioxol-4-one

To a stirred solution of 0.34 g (1.30 mmol) of Rh(acac)(eth)₂ and 1.17 g (3.24 mmol) of (R)-MonoPhos in 200 mL of ethanol were added 10.0 g (64.94 mmol) of (3aR,6aR)-2,2-dimethyl-3a,6a-dihydro-4H-cyclopenta[d][1,3]dioxol-4-one and 17.4 g (129.85 mmol) of potassium ethenyltrifluoroborate. The mixture was stirred at 80° C. for 2 h under N₂ atmosphere and filtered; the filter cake was washed with three 30 mL portions of ethanol. The combined filtrates were concentrated; the residue was diluted with 50 mL of water, and extracted with three 50 mL portions of ethyl acetate. The combined organic extracts were washed with brine, and dried over anhydrous Na₂SO₄. After filtration, the filtrate was concentrated to afford a residue, which was purified by chromatography on silica gel column eluting with 0 to 3% gradient of ethyl acetate in petroleum ether to afford compound 1-1. ¹H NMR (400 MHz, CDCl₃) δ 5.84 (ddd, J=17.2, 10.6, 6.4 Hz, 1H), 5.25-5.06 (m, 2H), 4.65 (dt, J=5.4, 1.2 Hz, 1H), 4.21 (dd, J=5.2, 0.8 Hz, 1H), 3.18-3.07 (m, 1H), 2.85 (ddd, J=18.3, 8.6, 1.0 Hz, 1H), 2.38-2.25 (m, 1H), 1.48-1.44 (m, 3H), 1.36 (d, J=0.7 Hz, 3H).

### Step 2:

To a stirred solution of 18.7 mL (18.7 mmol, 1 M in THF) of lithium aluminum hydride in 60 mL THF was added 8.5 g (46.7 mmol) of compound 1-1 dropwise at −78° C. The mixture was stirred at −78° C. for 1 h and then quenched by addition of 0.7 mL of water, 0.7 mL of 15% NaOH solution and 2.1 mL of water sequentially at −78° C. The resulting mixture was filtered; the filter cake was washed with three 50 mL portions of ethyl acetate. The combined filtrates were dried over anhydrous Na₂SO₄. After filtration, the filtrate was concentrated to afford a residue, which was purified by chromatography on silica gel column eluting with 0 to 3% gradient of ethyl acetate in petroleum ether to give compound 1-2. ¹H NMR (400 MHz, CDCl₃) δ 5.76 (ddd, J=17.2, 10.5, 6.5 Hz, 1H), 5.14-5.03 (m, 2H), 4.49 (d, J=3.2 Hz, 2H), 4.12-4.03 (m, 1H), 2.81-2.71 (m, 1H), 2.36 (s, 1H), 1.99-1.83 (m, 2H), 1.55-1.49 (m, 3H), 1.37 (d, J=0.7 Hz, 3H).

### Step 3:

To a stirred solution of 7.3 g (39.67 mmol) of compound 1-2 and 31.3 g (396.0 mmol) of pyridine in 120 mL of DCM was added 16.8 g (59.55 mmol) trifluoromethanesulfonic anhydride dropwise at 0° C. The reaction mixture was stirred at 0° C. for 1 h and then quenched by addition of 20 mL of water at 0° C. It was extracted with three 60 mL portions of DCM. The combined organic extracts were washed with brine, and dried over anhydrous Na₂SO₄. After filtration, the filtrate was concentrated to give a residue, which was purified by chromatography on silica gel column eluting with 0 to 2% gradient of ethyl acetate in petroleum ether to afford compound 1-3. ¹H NMR (400 MHz, CDCl₃) δ 5.78 (ddd, J=17.1, 10.6, 6.2 Hz, 1H), 5.21-5.07 (m, 2H), 5.03 (dt, J=8.1, 5.4 Hz, 1H), 4.65 (t, J=5.5 Hz, 1H), 4.53 (dd, J=6.0, 2.0 Hz, 1H), 2.95-2.85 (m, 1H), 2.40 (dt, J=13.2, 7.6 Hz, 1H), 2.15-2.04 (m, 1H), 1.56 (s, 3H), 1.36 (s, 3H).

### Step 4:

To a stirred solution of 10.0 g (65.1 mmol) of 4-chloro-7H-pyrrolo[2,3-d]pyrimidine in 120 mL THF was added 7.3 g (65.1 mmol) potassium tert-butoxide in portions at room temperature. The reaction mixture was stirred at rt for 1 h and concentrated under vacuum. The residue was purified by trituration with 120 mL isopropyl ether. The solids were collected by filtration and washed with three 50 mL portions of isopropyl ether to give potassium 4-chloro-7H-pyrrolo[2,3-d]pyrimidin-7-ide salt.

To a stirred solution of 7.0 g (22.2 mmol) of the above potassium 4-chloro-7H-pyrrolo[2,3-d]pyrimidin-7-ide in 80 mL DMF was added a solution of 5.07 g (26.6 mmol) of compound 1-3 in 20 mL of DMF dropwise at 0° C. The reaction mixture was stirred at rt for 2 h and quenched by slow addition of water at 0° C. The mixture was extracted with three 50 mL portions of ethyl acetate. The combined organic extracts were washed with brine, and dried over Na₂SO₄. After filtration, the filtrate was concentrated to afford a residue, which was purified by chromatography on silica gel column eluting with 0 to 15% gradient of ethyl acetate in petroleum ether to afford compound 1-4. LC-MS: m/e=320 [M+H]⁺.

### Step 5:

A solution of 5.0 g (15.6 mmol) of compound 1-4 in 60 mL of NH₃.H₂O and 60 mL of THF in a sealed tube was stirred at 110° C. overnight. It was cooled to rt, diluted with 50 mL water, and extracted with three 80 mL portions of ethyl acetate. The combined organic extracts were washed with brine and dried over Na₂SO₄. After filtration, the filtrate was concentrated to afford a residue, which was purified by chromatography on silica gel column eluting with 0 to 75% gradient of ethyl acetate in petroleum ether to give compound 1-5. LC-MS: m/e=301 [M+H]⁺.

### Step 6:

To a stirred solution of 0.27 g (0.88 mmol) of compound 1-5 in 5 mL of THF was added 8.1 mL (0.5 M, 4.1 mmol) of 9-borabicyclo[3.3.1]nonane dropwise at RT under argon atmosphere. The mixture was stirred at 50° C. for 1 h and cooled to RT. To the above mixture was added a solution of 0.85 g (4.0 mmol) in 2 mL of H₂O dropwise, 0.059 g (0.08 mmol) of Pd(dppf)Cl₂ and 0.20 g (0.80 mmol) of compound 3 at RT. The mixture was stirred at 50° C. for 1 h, diluted with water, and extracted with three 20 mL portions of EA. The combined organic layers were washed with brine and dried over anhydrous Na₂SO₄. After filtration, the filtrate was concentrated; the residue was purified by silica gel column chromatography eluting with 4% MeOH in DCM to afford a crude product, which was purified by reverse flash chromatography [column, C18 silica gel; ACN, NH₄HCO₃ (0.5%) in water, 55% to 60% gradient in 10 min; detector, UV 254 nm] to afford compound 1-6. LC-MS: m/e=471 [M+H]⁺.

### Step 7:

To a stirred solution of 0.20 g (0.43 mmol) of compound 1-6 in 8 mL of THF was added 2 mL of concentrated HCl. The reaction mixture was stirred at rt for 4 h and concentrated. The residue was diluted with 2 mL of water, adjusted to pH 7 with saturated sodium bicarbonate and concentrated. The residue was purified by Prep-HPLC [Column: Shim-pack XR-ODS (50*3.0 mm) 2.2 m; Mobile phase: A: 0.05% Trifluoroacetic acid in Water, B: 0.05% Trifluoroacetic acid in Acetonitrile; 95:5 to 0:100(A:B) over 2 min, 0:100(A:B) for 0.7 min, 0:100 to 95:5(A:B) over 0.05 min. Flow Rate: 1.2 mL/min. UV. Detection: 190-400 nm)] to give compound 1-7. LC-MS: m/e=431 [M+H]⁺.

Using the procedures outlined in Method 1, steps 6-7, the following analogs in Table 2 were made from compound 1-5 by employing the requisite aryl halide. Other compounds of Formula 1 may be prepared in a similar way. Diastereoisomers 1-10 and 1-11 were separated by HPLC, and the stereochemistry of the cyclopropyl ring is arbitrarily assigned.

TABLE 2
Synthesis of heterocyclic analogs as examples

| | | LCMS |
|---|---|---|
| Aryl Halide Employed | Example Structure | [M + H]⁺ |
| 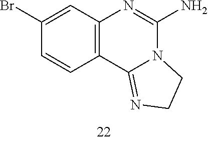 [SMILES: NC1=Nc2cc(Br)ccc2C2=NCCN12] | 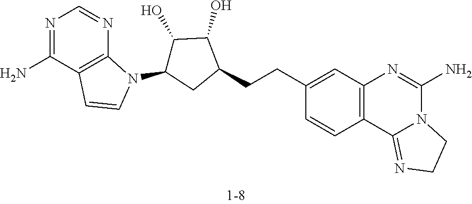 [SMILES: NC1=Nc2cc(CC[C@H]3C[C@@H](n4ccc5c(N)ncnc54)[C@H](O)[C@@H]3O)ccc2C2=NCCN12] | 447 |
| 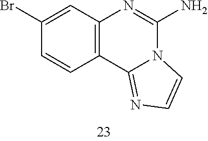 [SMILES: Nc1nc2cc(Br)ccc2c2nccn12] | 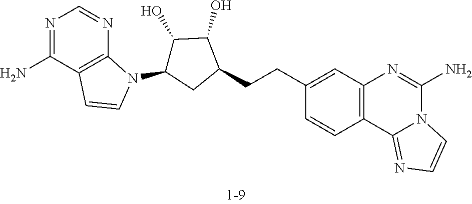 [SMILES: Nc1ncnc2c1ccn2[C@@H]1C[C@H](CCc2ccc3c(c2)nc(N)n2ccnc32)[C@@H](O)[C@H]1O] | 445 |
| 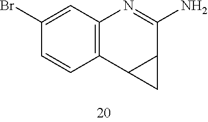 [SMILES: NC1=Nc2cc(Br)ccc2C2CC12] | 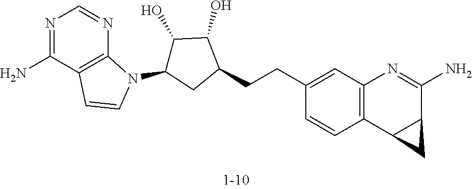 [SMILES: NC1=Nc2cc(CC[C@H]3C[C@@H](n4ccc5c(N)ncnc54)[C@H](O)[C@@H]3O)ccc2[C@@H]2C[C@H]12] | 419 |
| 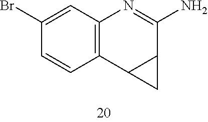 [SMILES: NC1=Nc2cc(Br)ccc2C2CC12] | 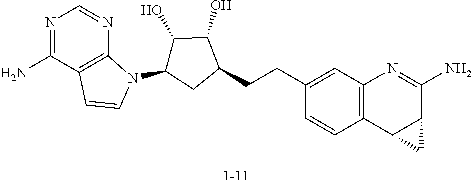 [SMILES: NC1=Nc2cc(CC[C@H]3C[C@@H](n4ccc5c(N)ncnc54)[C@H](O)[C@@H]3O)ccc2[C@H]2C[C@@H]12] | 419 |
| 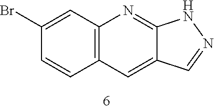 [SMILES: Brc1ccc2cc3cn[nH]c3nc2c1] | 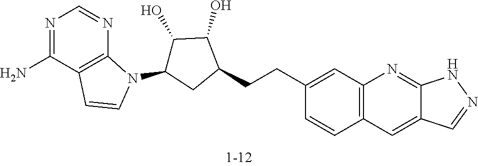 [SMILES: Nc1ncnc2c1ccn2[C@@H]1C[C@H](CCc2ccc3cc4cn[nH]c4nc3c2)[C@@H](O)[C@H]1O] | 430 |
| 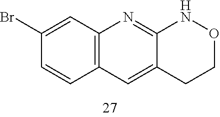 [SMILES: Brc1ccc2cc3c(nc2c1)NOCC3] | 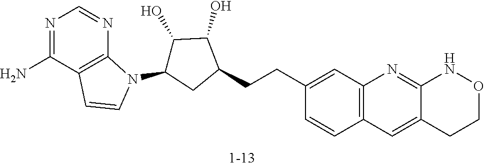 [SMILES: Nc1ncnc2c1ccn2[C@@H]1C[C@H](CCc2ccc3cc4c(nc3c2)NOCC4)[C@@H](O)[C@H]1O] | 447 |
| 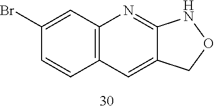 [SMILES: Brc1ccc2cc3c(nc2c1)NOC3] | 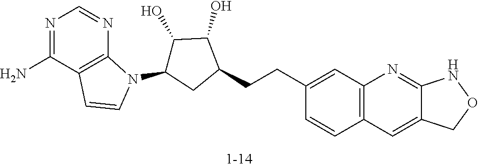 [SMILES: Nc1ncnc2c1ccn2[C@@H]1C[C@H](CCc2ccc3cc4c(nc3c2)NOC4)[C@@H](O)[C@H]1O] | 433 |
| 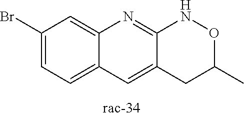 [SMILES: CC1Cc2cc3ccc(Br)cc3nc2NO1] | 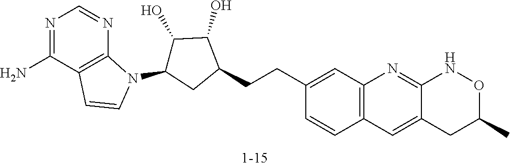 [SMILES: C[C@H]1Cc2cc3ccc(CC[C@H]4C[C@@H](n5ccc6c(N)ncnc65)[C@H](O)[C@@H]4O)cc3nc2NO1] | 461 |
| 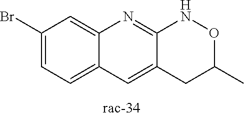 [SMILES: CC1Cc2cc3ccc(Br)cc3nc2NO1] | 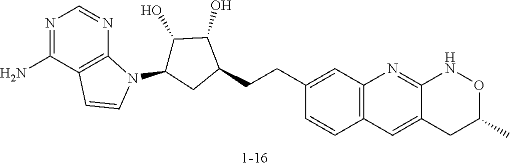 [SMILES: C[C@@H]1Cc2cc3ccc(CC[C@H]4C[C@@H](n5ccc6c(N)ncnc65)[C@H](O)[C@@H]4O)cc3nc2NO1] | 461 |
| 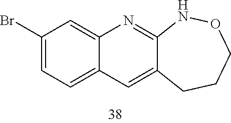 [SMILES: Brc1ccc2cc3c(nc2c1)NOCCC3] | 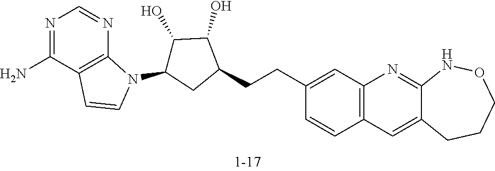 [SMILES: Nc1ncnc2c1ccn2[C@@H]1C[C@H](CCc2ccc3cc4c(nc3c2)NOCCC4)[C@@H](O)[C@H]1O] | 461 |
| 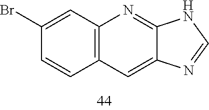 [SMILES: Brc1ccc2cc3nc[nH]c3nc2c1] | 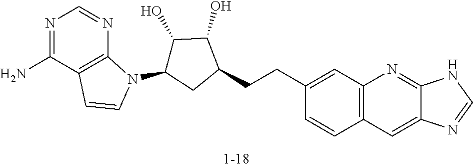 [SMILES: Nc1ncnc2c1ccn2[C@@H]1C[C@H](CCc2ccc3cc4nc[nH]c4nc3c2)[C@@H](O)[C@H]1O] | 430 |
| 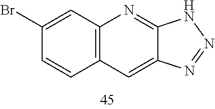 [SMILES: Brc1ccc2cc3nn[nH]c3nc2c1] | 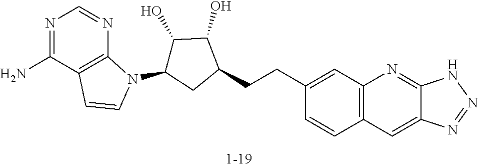 [SMILES: Nc1ncnc2c1ccn2[C@@H]1C[C@H](CCc2ccc3cc4nn[nH]c4nc3c2)[C@@H](O)[C@H]1O] | 431 |
| 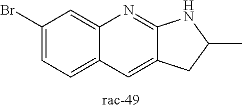 | 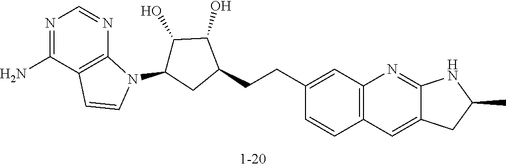 [SMILES: C[C@H]1Cc2cc3ccc(CC[C@H]4C[C@@H](n5ccc6c(N)ncnc65)[C@H](O)[C@@H]4O)cc3nc2N1] | 445 |
| 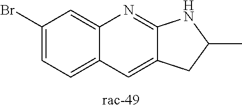 | 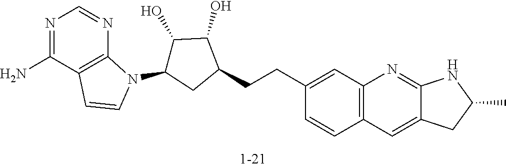 [SMILES: C[C@@H]1Cc2cc3ccc(CC[C@H]4C[C@@H](n5ccc6c(N)ncnc65)[C@H](O)[C@@H]4O)cc3nc2N1] | 445 |
| 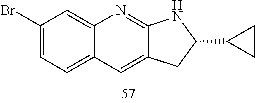 [SMILES: Brc1ccc2cc3c(nc2c1)N[C@H](C1CC1)C3] | 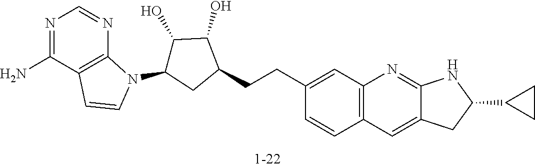 [SMILES: Nc1ncnc2c1ccn2[C@@H]1C[C@H](CCc2ccc3cc4c(nc3c2)N[C@H](C2CC2)C4)[C@@H](O)[C@H]1O] | 471 |
| 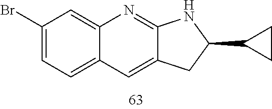 [SMILES: Brc1ccc2cc3c(nc2c1)N[C@@H](C1CC1)C3] | 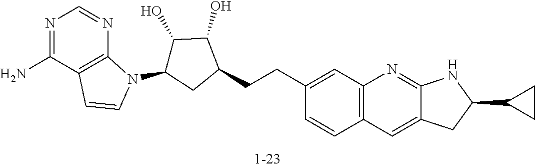 [SMILES: Nc1ncnc2c1ccn2[C@@H]1C[C@H](CCc2ccc3cc4c(nc3c2)N[C@@H](C2CC2)C4)[C@@H](O)[C@H]1O] | 471 |
| 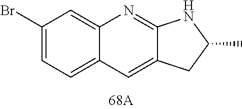 | 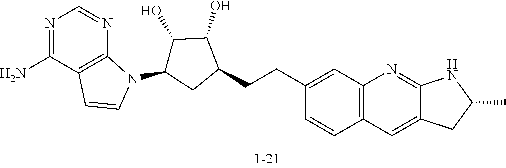 [SMILES: C[C@@H]1Cc2cc3ccc(CC[C@H]4C[C@@H](n5ccc6c(N)ncnc65)[C@H](O)[C@@H]4O)cc3nc2N1] | 445 |
| 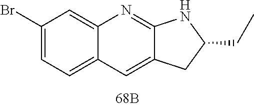 [SMILES: CC[C@@H]1Cc2cc3ccc(Br)cc3nc2N1] | 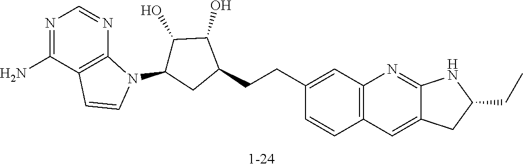 [SMILES: CC[C@@H]1Cc2cc3ccc(CC[C@H]4C[C@@H](n5ccc6c(N)ncnc65)[C@H](O)[C@@H]4O)cc3nc2N1] | 459 |
| 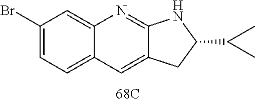 [SMILES: CC(C)[C@@H]1Cc2cc3ccc(Br)cc3nc2N1] | 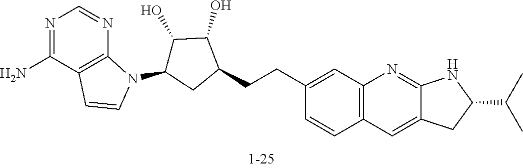 [SMILES: CC(C)[C@@H]1Cc2cc3ccc(CC[C@H]4C[C@@H](n5ccc6c(N)ncnc65)[C@H](O)[C@@H]4O)cc3nc2N1] | 473 |
| 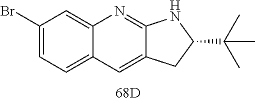 [SMILES: CC(C)(C)[C@@H]1Cc2cc3ccc(Br)cc3nc2N1] | 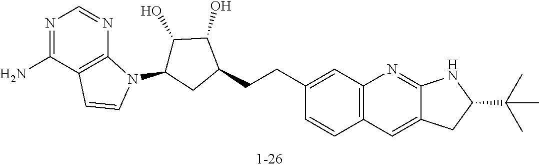 [SMILES: CC(C)(C)[C@@H]1Cc2cc3ccc(CC[C@H]4C[C@@H](n5ccc6c(N)ncnc65)[C@H](O)[C@@H]4O)cc3nc2N1] | 487 |
| 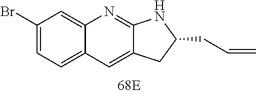 [SMILES: C=CC[C@@H]1Cc2cc3ccc(Br)cc3nc2N1] | 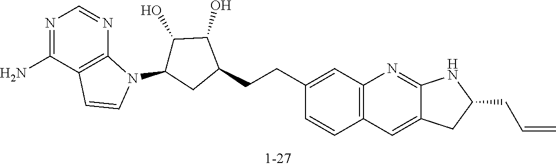 [SMILES: C=CC[C@@H]1Cc2cc3ccc(CC[C@H]4C[C@@H](n5ccc6c(N)ncnc65)[C@H](O)[C@@H]4O)cc3nc2N1] | 471 |
| 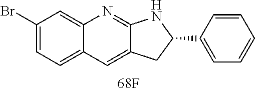 [SMILES: Brc1ccc2cc3c(nc2c1)N[C@H](c1ccccc1)C3] | 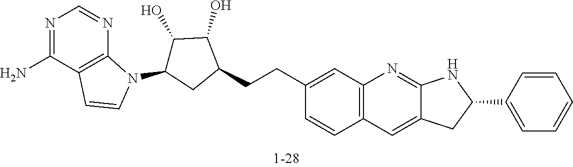 [SMILES: Nc1ncnc2c1ccn2[C@@H]1C[C@H](CCc2ccc3cc4c(nc3c2)N[C@H](c2ccccc2)C4)[C@@H](O)[C@H]1O] | 507 |
| 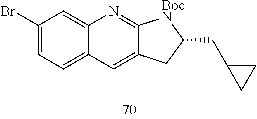 [SMILES: CC(C)(C)OC(=O)N1c2nc3cc(Br)ccc3cc2C[C@H]1CC1CC1] | 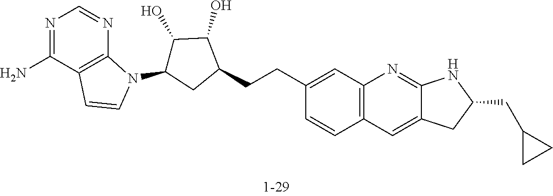 [SMILES: Nc1ncnc2c1ccn2[C@@H]1C[C@H](CCc2ccc3cc4c(nc3c2)N[C@H](CC2CC2)C4)[C@@H](O)[C@H]1O] | 485 |
| 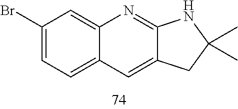 [SMILES: CC1(C)Cc2cc3ccc(Br)cc3nc2N1] | 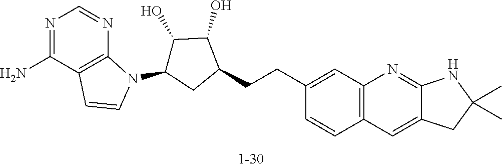 [SMILES: CC1(C)Cc2cc3ccc(CC[C@H]4C[C@@H](n5ccc6c(N)ncnc65)[C@H](O)[C@@H]4O)cc3nc2N1] | 459 |
| 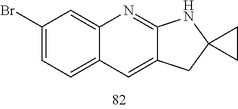 [SMILES: Brc1ccc2cc3c(nc2c1)NC1(CC1)C3] | 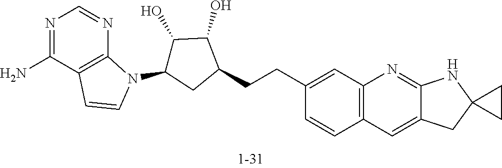 [SMILES: Nc1ncnc2c1ccn2[C@@H]1C[C@H](CCc2ccc3cc4c(nc3c2)NC2(CC2)C4)[C@@H](O)[C@H]1O] | 471 |
| 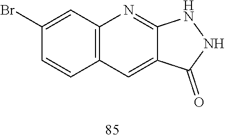 [SMILES: O=c1[nH][nH]c2nc3cc(Br)ccc3cc12] | 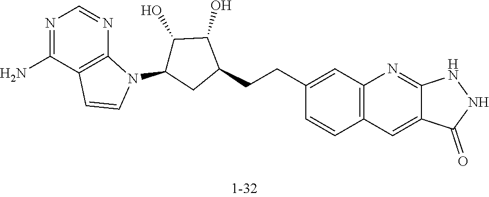 [SMILES: Nc1ncnc2c1ccn2[C@@H]1C[C@H](CCc2ccc3cc4c(=O)[nH][nH]c4nc3c2)[C@@H](O)[C@H]1O] | 446 |
| 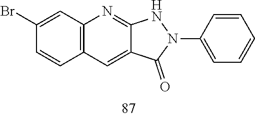 [SMILES: O=c1c2cc3ccc(Br)cc3nc2[nH]n1-c1ccccc1] | 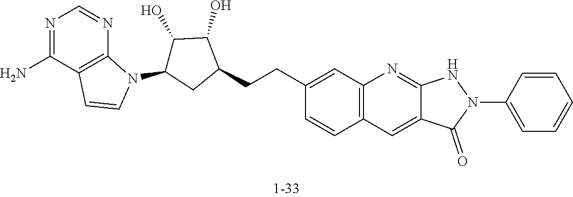 [SMILES: Nc1ncnc2c1ccn2[C@@H]1C[C@H](CCc2ccc3cc4c(=O)n(-c5ccccc5)[nH]c4nc3c2)[C@@H](O)[C@H]1O] | 522 |
| 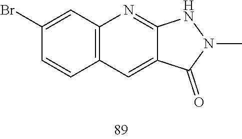 [SMILES: Cn1[nH]c2nc3cc(Br)ccc3cc2c1=O] | 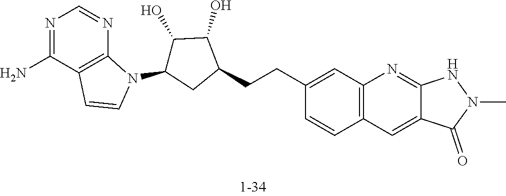 [SMILES: Cn1[nH]c2nc3cc(CC[C@H]4C[C@@H](n5ccc6c(N)ncnc65)[C@H](O)[C@@H]4O)ccc3cc2c1=O] | 460 |
| 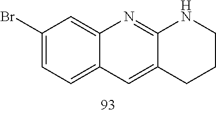 [SMILES: Brc1ccc2cc3c(nc2c1)NCCC3] | 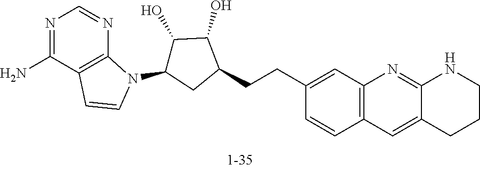 [SMILES: Nc1ncnc2c1ccn2[C@@H]1C[C@H](CCc2ccc3cc4c(nc3c2)NCCC4)[C@@H](O)[C@H]1O] | 445 |
| 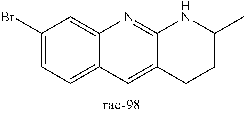 [SMILES: CC1CCc2cc3ccc(Br)cc3nc2N1] | 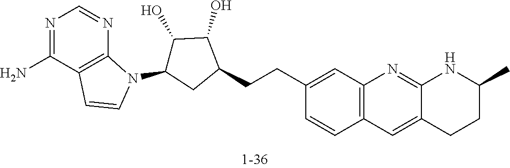 [SMILES: C[C@H]1CCc2cc3ccc(CC[C@H]4C[C@@H](n5ccc6c(N)ncnc65)[C@H](O)[C@@H]4O)cc3nc2N1] | 459 |
| 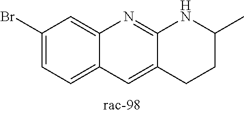 [SMILES: CC1CCc2cc3ccc(Br)cc3nc2N1] | 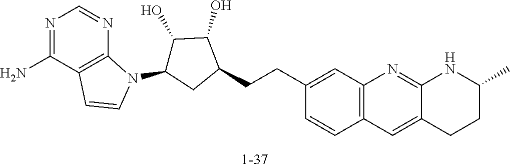 [SMILES: C[C@@H]1CCc2cc3ccc(CC[C@H]4C[C@@H](n5ccc6c(N)ncnc65)[C@H](O)[C@@H]4O)cc3nc2N1] | 459 |
| 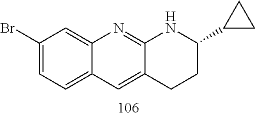 [SMILES: Brc1ccc2cc3c(nc2c1)N[C@H](C1CC1)CC3] | 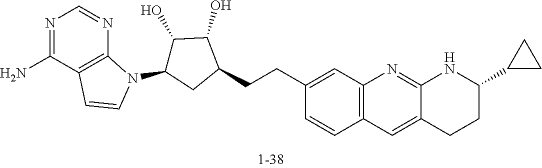 [SMILES: Nc1ncnc2c1ccn2[C@@H]1C[C@H](CCc2ccc3cc4c(nc3c2)N[C@H](C2CC2)CC4)[C@@H](O)[C@H]1O] | 485 |
| 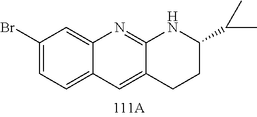 [SMILES: CC(C)[C@@H]1CCc2cc3ccc(Br)cc3nc2N1] | 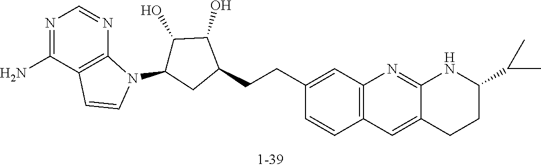 [SMILES: CC(C)[C@@H]1CCc2cc3ccc(CC[C@H]4C[C@@H](n5ccc6c(N)ncnc65)[C@H](O)[C@@H]4O)cc3nc2N1] | 487 |
| 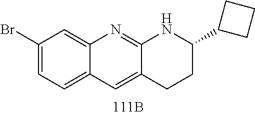 [SMILES: Brc1ccc2cc3c(nc2c1)N[C@H](C1CCC1)CC3] | 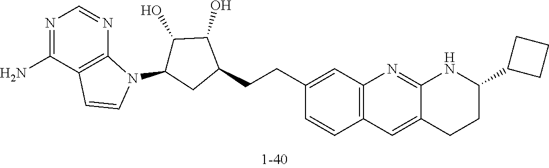 [SMILES: Nc1ncnc2c1ccn2[C@@H]1C[C@H](CCc2ccc3cc4c(nc3c2)N[C@H](C2CCC2)CC4)[C@@H](O)[C@H]1O] | 499 |
| 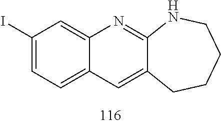 [SMILES: Ic1ccc2cc3c(nc2c1)NCCCC3] | 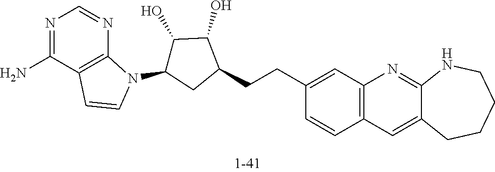 [SMILES: Nc1ncnc2c1ccn2[C@@H]1C[C@H](CCc2ccc3cc4c(nc3c2)NCCCC4)[C@@H](O)[C@H]1O] | 459 |
| 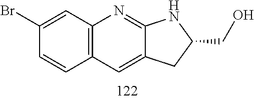 [SMILES: OC[C@@H]1Cc2cc3ccc(Br)cc3nc2N1] | 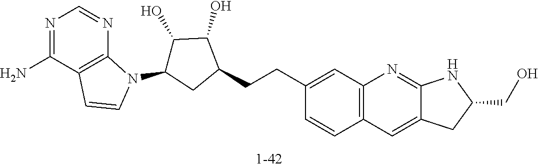 [SMILES: Nc1ncnc2c1ccn2[C@@H]1C[C@H](CCc2ccc3cc4c(nc3c2)N[C@H](CO)C4)[C@@H](O)[C@H]1O] | 461 |
| 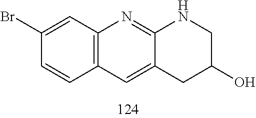 [SMILES: OC1CNc2nc3cc(Br)ccc3cc2C1] | 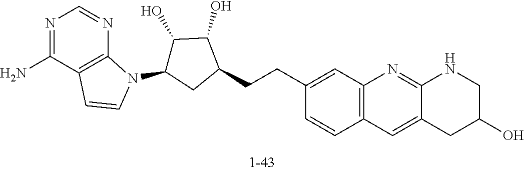 [SMILES: Nc1ncnc2c1ccn2[C@@H]1C[C@H](CCc2ccc3cc4c(nc3c2)NCC(O)C4)[C@@H](O)[C@H]1O] | 461 |

Method 2:

### Example 2: Synthesis of (1R,2S,3R,5S)-3-(4-amino-7H-pyrrolo[2,3-d]pyrimidin-7-yl)-5-(2-(3,3-dimethyl-2,3-dihydro-1H-pyrrolo[2,3-b]quinolin-7-yl)ethyl)cyclopentane-1,2-diol

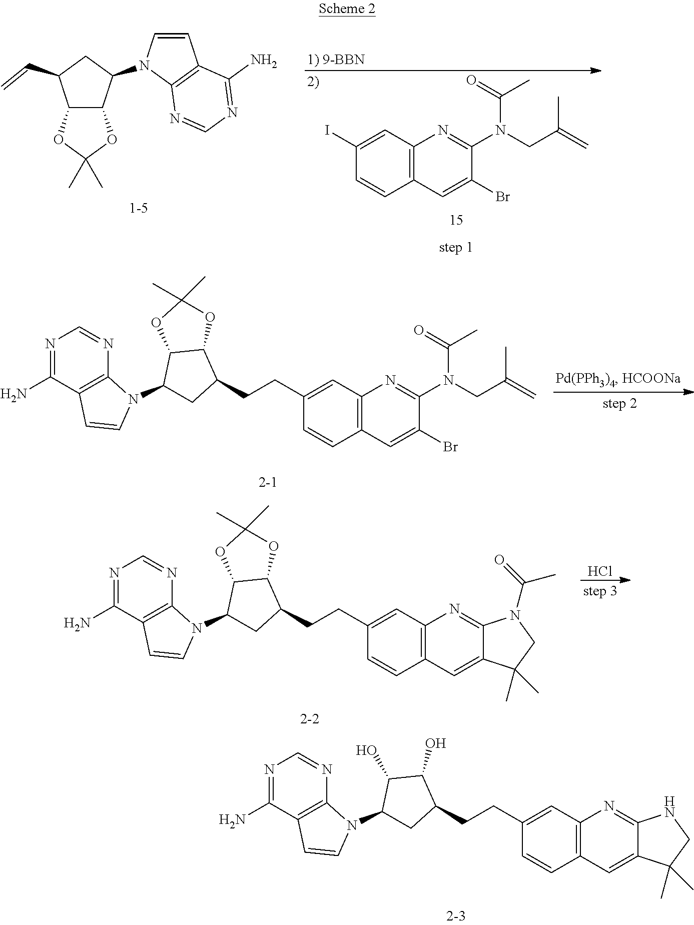 [SMILES: C=C[C@H]1C[C@@H](n2ccc3c(N)ncnc32)[C@@H]2OC(C)(C)O[C@@H]21 ; C=C(C)CN(C(C)=O)c1nc2cc(I)ccc2cc1Br ; C=C(C)CN(C(C)=O)c1nc2cc(CC[C@H]3C[C@@H](n4ccc5c(N)ncnc54)[C@@H]4OC(C)(C)O[C@H]34)ccc2cc1Br ; CC(=O)N1CC(C)(C)c2cc3ccc(CC[C@H]4C[C@@H](n5ccc6c(N)ncnc65)[C@@H]5OC(C)(C)O[C@H]45)cc3nc21 ; Cl ; CC1(C)CNc2nc3cc(CC[C@H]4C[C@@H](n5ccc6c(N)ncnc65)[C@H](O)[C@@H]4O)ccc3cc21]

### Step 1:

Following the procedure described in Method 1, step 6, compound 2-1 was prepared from compound 1-5 using intermediate 15 as the coupling partner. LC-MS: m/e=619 [M+H]⁺.

### Step 2:

To a stirred mixture of 150 mg (0.24 mmol) of compound 2-1 and 1009 mg (3.63 mmol) of tetrabutyl ammonium chloride in DMF were added 33 mg (0.48 mmol) of sodium formate, 59.58 mg (0.73 mmol) of NaOAc and 364 mg (0.32 mmol) of Pd(PPh₃)₄ in portions at RT under argon atmosphere. The mixture was stirred at 80° C. for 2 h under argon atmosphere and cooled to RT. The mixture was filtered, and the filter cake was washed with ethyl acetate (3×20 mL).

The combined organic layers were washed with water and dried over anhydrous Na₂SO₄. It was filtered and the filtrate was concentrated. The residue was purified by silica gel column chromatography eluting with 10% MeOH in DCM to afford compound 2-2. LC-MS: m/e=541 [M+H]⁺.

### Step 3:

Following the procedure described in Method 1, step 7, compound 2-3 was prepared from compound 2-2. LC-MS: m/e=459 [M+H]⁺.

### Method 3:

### Example 3: Synthesis of (2R,3R,4S,5R)-2-(4-amino-7H-pyrrolo[2,3-d]pyrimidin-7-yl)-5-(2-(2,3-dihydro-1H-pyrrolo[2,3-b]quinolin-7-yl)ethyl)tetrahydrofuran-3,4-diol

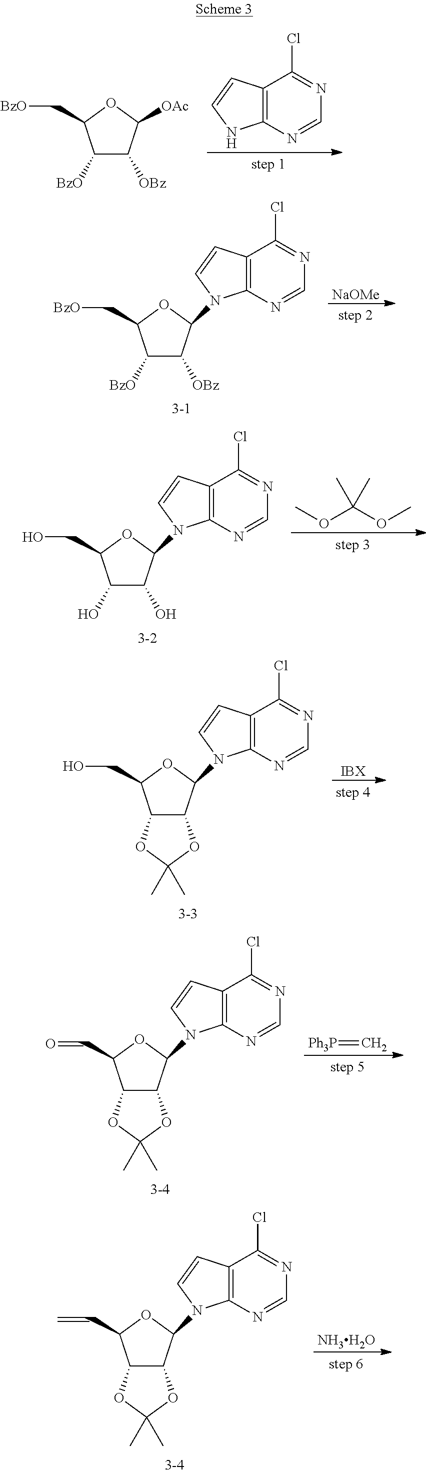

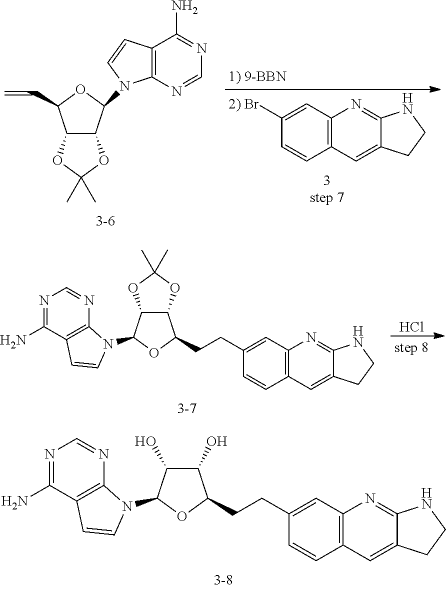

### Step 1:

To a solution of 10.0 g (65.4 mmol) of 4-chloro-7H-pyrrolo[2,3-d]pyrimidine in 250 mL of acetonitrile was added 16.0 g (78.5 mmol) of BSA. The resulting solution was stirred at rt for 40 min. After addition of 49.5 g (98.1 mmol) of (2S,3R,4R,5R)-2-(acetyloxy)-4-(benzoyloxy)-5-[(benzoyloxy)methyl]oxolan-3-yl benzoate and 22.0 g (98.1 mmol) of TMSOTf, the mixture was stirred at 85° C. for 2 h. The reaction was then quenched by addition of 500 mL of ice water and extracted with three 150 mL portions of ethyl acetate. The combined organic extracts were washed with 150 mL of brine, and dried over anhydrous sodium sulfate. After filtration, the filtrate was concentrated to give a residue, which was purified by chromatography on a silica gel column eluting with 0 to 5% gradient of ethyl acetate in petroleum ether to afford compound 3-1. LC-MS: m/e=598 [M+H]⁺.

### Step 2:

To a solution of 24.0 g (40.1 mmol) of compound 3-1 in 200 mL of methanol and 20 mL of dichloromethane was added 1.1 mg (0.02 mmol) of sodium methoxide. The solution was stirred at rt for 60 min; the solution was adjusted to pH 5˜6 with 1N HCl solution. Then the mixture was concentrated; the solids were collected by filtration to afford compound 3-2. LC-MS: m/e=286 [M+H]⁺.

### Step 3:

To a solution of 10.0 g (35.0 mmol) of compound 3-2 in 200 mL of acetone was added 600 mg (3.48 mmol) of TsOH and 11.0 g (105.6 mmol) of 2,2-dimethoxypropane. The mixture was stirred at rt for 2 h. The reaction was then quenched by addition of 150 mL of water and extracted with three 150 mL portions of DCM. The combined organic extracts were washed with 150 mL of brine, and dried over anhydrous magnesium sulfate. After filtration, the filtrate was concentrated to afford compound 3-3, which was used in the next step without further purification. LC-MS: m/e=326 [M+H]⁺.

### Step 4:

To a solution of 10.0 g (30.7 mmol) of compound 3-3 in 110 mL of acetonitrile was added 12.9 g (46.1 mmol) of IBX. The mixture was stirred at 50° C. for 16 h and then cooled with an ice water bath. After filtration, the filtrate was concentrated to afford crude compound 3-4, which was used in the next step without further purification. LC-MS: m/e=324 [M+H]⁺.

### Step 5:

To a solution of 33.1 g (92.7 mmol) of bromo(methyl)triphenyl-lambda5-phosphane in 200 mL of THF was added 85 mL (85.0 mmol) of 1 M t-BuOK solution in THF. Then the mixture was stirred at 0° C. for 1 h, a solution of 10.0 g (30.9 mmol) of compound 3-4 in 10 mL of THF was introduced. The mixture was stirred at 0° C. for additional 1 h and then quenched by addition of 300 mL of saturated NH₄Cl solution. It was extracted with three 150 mL portions of ethyl acetate, the combined organic extracts were washed with 150 mL of brine and dried over anhydrous sodium sulfate. After filtration, the filtrate was concentrated to afford a residue, which was purified by chromatography on a silica gel column eluting with 0 to 3% gradient of ethyl acetate in petroleum ether to afford 4.3 g of compound 3-5. LC-MS: m/e=322 [M+H]⁺.

### Step 6:

To a solution of 5.5 g (17.1 mmol) of compound 3-5 in 30 mL of 1,4-dioxane was added 30 mL of ammonia. The mixture was stirred at 100° C. for 20 h. Then the mixture was concentrated to afford 3.5 g of compound 3-6, which was used in the next step without further purification. LC-MS: m/e=303 [M+H]⁺.

### Step 7:

Compound 3-7 was prepared from compound 3-6, using the similar procedure described in Method 1, Step 6, by employing the intermediate 3 as the coupling partner. LC-MS: m/e=473 [M+H]⁺.

### Step 8:

Compound 3-8 was prepared from compound 3-7, using the similar procedure described in Method 1, Step 7. LC-MS (Shimadzu, Column: Shim-pack XR-ODS, 3.0*50 mm, 2.2 μm; Mobile Phase A: water/0.05% TFA, Mobile Phase B: ACN/0.05% TFA; Flow rate: 1.2 mL/min; Gradient: 5% B to 100% B in 2.0 min, hold 0.7 min; 190-400 nm): m/e=433 [M+H]⁺.

Using the procedures outlined in Method 1, Steps 6-7, the following analogs in Table 3 were made from compound 3-6 by employing the requisite aryl halide. Other compounds of Formula 1 may be prepared in a similar way.

TABLE 3
Synthesis of heterocyclic analogs as examples

| | | LCMS |
|---|---|---|
| Aryl Halide Employed | Example Structure | [M + H]⁺ |
| 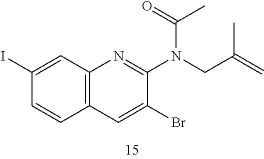 [SMILES: C=C(C)CN(C(C)=O)c1nc2cc(I)ccc2cc1Br] | 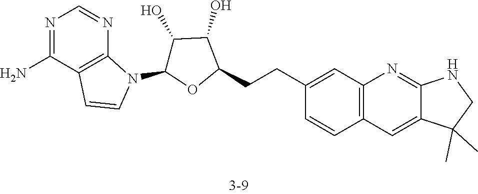 [SMILES: CC1(C)CNc2nc3cc(CC[C@H]4O[C@@H](n5ccc6c(N)ncnc65)[C@H](O)[C@@H]4O)ccc3cc21] | 461 |
| 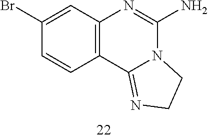 [SMILES: NC1=Nc2cc(Br)ccc2C2=NCCN12] | 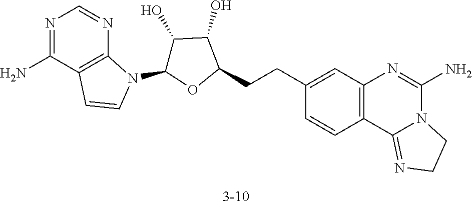 [SMILES: NC1=Nc2cc(CC[C@H]3O[C@@H](n4ccc5c(N)ncnc54)[C@H](O)[C@@H]3O)ccc2C2=NCCN12] | 449 |
| 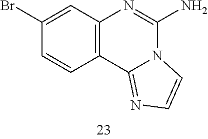 [SMILES: Nc1nc2cc(Br)ccc2c2nccn12] | 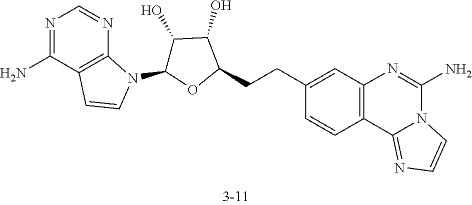 [SMILES: Nc1ncnc2c1ccn2[C@@H]1O[C@H](CCc2ccc3c(c2)nc(N)n2ccnc32)[C@@H](O)[C@H]1O] | 447 |
| 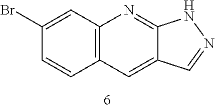 [SMILES: Brc1ccc2cc3cn[nH]c3nc2c1] | 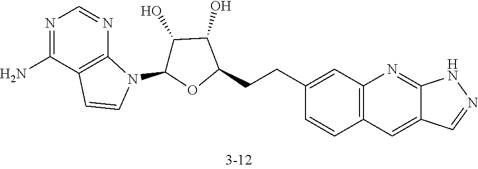 [SMILES: Nc1ncnc2c1ccn2[C@@H]1O[C@H](CCc2ccc3cc4cn[nH]c4nc3c2)[C@@H](O)[C@H]1O] | 432 |
| 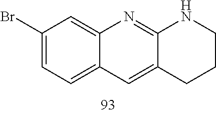 [SMILES: Brc1ccc2cc3c(nc2c1)NCCC3] | 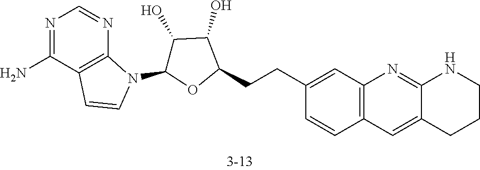 [SMILES: Nc1ncnc2c1ccn2[C@@H]1O[C@H](CCc2ccc3cc4c(nc3c2)NCCC4)[C@@H](O)[C@H]1O] | 447 |

### Method 4:

### Example 4: Synthesis of (1S,2R,3S,5R)-3-(2-(1H-pyrrolo[2,3-b]quinolin-7-yl)ethyl-5-(4-amino-7H-pyrrolo[2,3-d]pyrimidin-7-yl)cyclopentane-1,2-diol

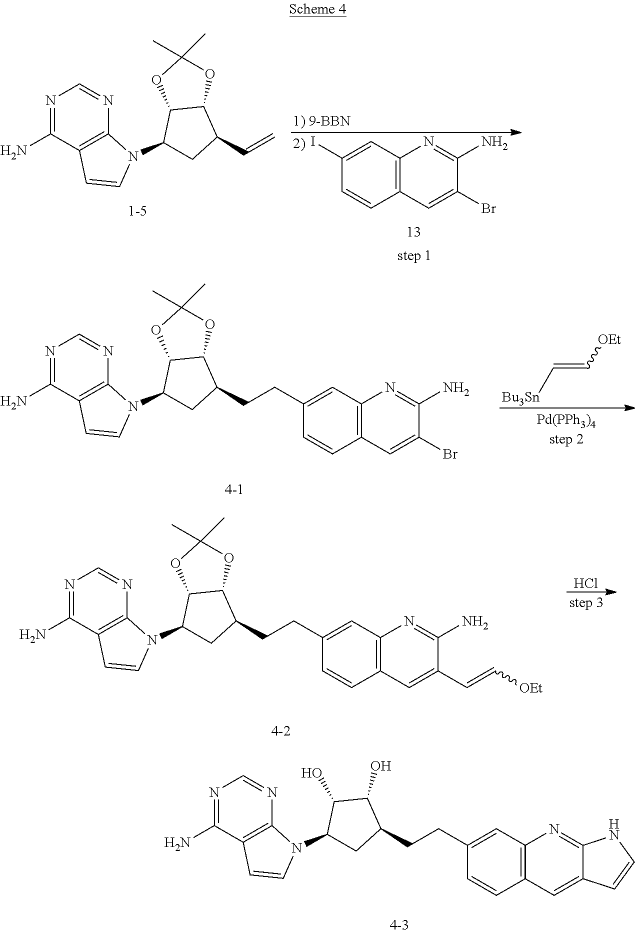

### Step 1:

Using the procedure described in Method 1, Step 6, compound 4-1 was prepared from compound 1-5 by employing intermediate 13 as the coupling partner. LC-MS: m/e=523, 525 [M+H]⁺.

### Step 2:

Into a 20 mL microwave vial were added 85 mg (0.16 mmol) of compound 4-1, 11 mg (0.01 mmol) of Pd(PPh₃)₄, 76 mg (0.59 mmol) of DIEA and 117 mg (0.33 mmol) of Z-(1)-ethoxy-(2)-(tributylstannyl)ethylene and 5 mL of toluene. The mixture was irradiated with microwave radiation at 180° C. for 20 min and concentrated to afford compound 4-2, which was used in the next step without further purification. LC-MS: m/e=515 [M+H]⁺.

### Step 3:

Using the procedure described in Method 1, Step 7, compound 4-3 was prepared from compound 4-2 similarly. LC-MS: m/e=429 [M+H]⁺.

### Synthesis of Intermediates

### 1. Synthesis of Intermediate 3:

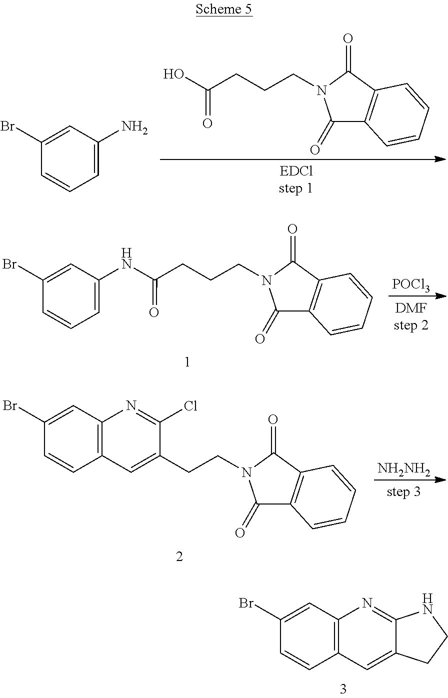

### Step 1:

To a stirred solution of 0.87 g (5.0 mmol) of m-bromoaniline and 2.59 g (11.1 mmol) of 4-phthalimidobutyric acid in 50 mL of CHCl₃ was added 2.32 g (12.1 mmol) of EDCI in portions at RT under argon atmosphere. The mixture was stirred at RT for 2 h, quenched with water, and extracted with three 50 mL portions of ethyl acetate. The combined organic layers were washed with brine and dried over anhydrous Na₂SO₄. After filtration, the filtrate was concentrated; the residue was purified by silica gel column chromatography eluting with 10% ethyl acetate in dichloromethane to afford compound 1. LC-MS: m/e=387 [M+H]⁺.

### Step 2:

Into a 50 mL three-neck round bottom flask were added 1.09 g (7.1 mmol) of phosphorus oxychloride and 0.14 g (1.9 mmol) of dimethylformamide at 10° C. under nitrogen atmosphere. The mixture was stirred at 10° C. for additional 30 min. To the above mixture was added 0.50 g (1.3 mmol) of compound 1 in portions at 10° C. The mixture was stirred at 80° C. for 12 h and cooled to 0° C. and quenched with water. The mixture was neutralized to pH 7 with saturated aqueous NaHCO₃ and extracted with three 30 mL portions of EA. The combined organic layers were washed with brine and dried over anhydrous Na₂SO₄. After filtration, the filtrate was concentrated, the residue was purified by silica gel column chromatography eluting with 1% EA in DCM to afford compound 2. LC-MS: m/e=415 [M+H]⁺.

### Step 3:

To a stirred solution of 1.50 g (3.6 mmol) of compound 2 in 80 mL of n-butanol was added 0.22 g (0.004 mmol) of NH₂NH₂.H₂O dropwise at 80° C. under argon atmosphere. The mixture was stirred at 100° C. for 12 h and cooled to RT. The mixture was diluted with water and extracted with three 30 mL portions of EA. The combined organic layers were washed with brine and dried over anhydrous Na₂SO₄. After filtration, the filtrate was concentrated; the residue was purified by silica gel column chromatography eluting with 1% EA in DCM to afford intermediate 3. LC-MS: m/e=249 [M+H]⁺.

### 2. Synthesis of Intermediate 6:

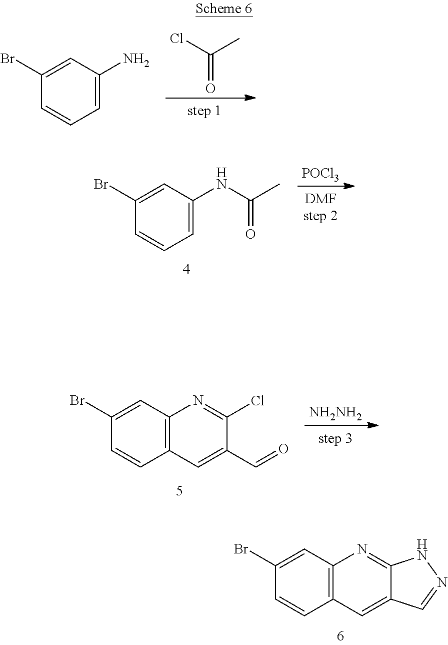

### Step 1:

To a stirred solution of 5.00 g (29.1 mmol) of m-bromoaniline in 150 mL of CHCl₃ were added 2.51 g (32.0 mmol) of acetyl chloride and 5.88 g (58.1 mmol) of Et₃N dropwise at 0° C. under argon atmosphere. The mixture was stirred at 0° C. for 1 h, quenched with water, and extracted with DCM. The combined organic layers were washed with brine and dried over anhydrous Na₂SO₄. After filtration, the filtrate was concentrated; the residue was purified by silica gel column chromatography eluting with 1% EA in DCM to afford compound 4. LC-MS: m/e=214 [M+H]⁺.

### Step 2:

Into a 50 mL three-neck round bottom flask were added 5.01 g (32.7 mmol) of phosphorus oxychloride and 1.02 g (14.0 mmol) of dimethylformamide at 10° C. under nitrogen atmosphere. The mixture was stirred at 10° C. for additional 30 min. To the above mixture was added 1.00 g (4.7 mmol) of 3-bromoacetanilide 4 in portions at 10° C. The mixture was stirred at 80° C. for 12 h and cooled to 0° C. The reaction was quenched with water, neutralized to pH 7 with saturated aqueous NaHCO₃, and extracted with three 30 mL portions of EA. The combined organic layers were washed with brine and dried over anhydrous Na₂SO₄. After filtration, the filtrate was concentrated; the residue was purified by silica gel column chromatography eluting with 10% ethyl acetate in petroleum ether to afford compound 5. LC-MS: m/e=270 [M+H]⁺.

### Step 3:

To a stirred solution of 0.19 g (0.70 mmol) of compound 5 in 20 mL of n-butanol were added 0.70 g (14.0 mmol) of N₂H₄.H₂O dropwise at RT under argon atmosphere. The mixture was stirred at 70° C. for 5 h and cooled to RT. It was diluted with water and extracted with three 30 mL portions of EA. The combined organic layers were washed with brine and dried over anhydrous Na₂SO₄. After filtration, the filtrate was concentrated; the residue was purified by silica gel column chromatography eluting with 5% MeOH in DCM to afford intermediate 6. LC-MS: m/e=248 [M+H]⁺.

### 3. Synthesis of Intermediate 15:

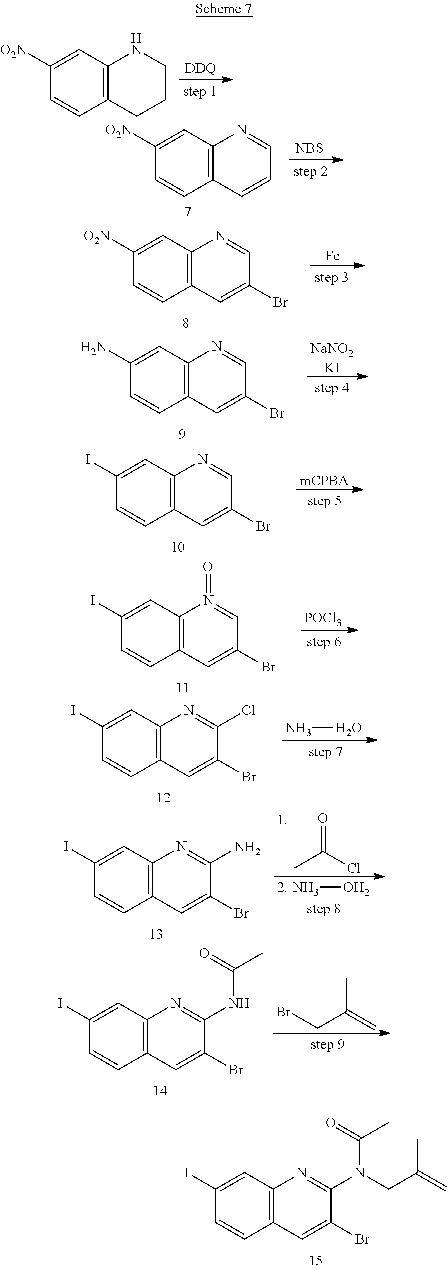

### Step 1:

To a stirred solution of 20.0 g (112 mmol) of 7-nitro-1,2,3,4-tetrahydroquinoline in 600 mL of dichloromethane was added 50.8 g (224 mmol) of 2,3-dichloro-5,6-dicyano-1,4-benzoquinone in several batches at 0° C. The mixture was stirred at room temperature for 3 h and filtered; the filter cake was washed with three 200 mL portions of dichloromethane. The filtrate was concentrated under vacuum to afford compound 7. LC-MS: m/e=175 [M+H]⁺.

### Step 2:

To a stirred solution of 23.0 g (132 mmol) of compound 7 in 180 mL of acetic acid was added 30.2 g (170 mmol) of NBS in several batches at room temperature. The reaction mixture was stirred at 110° C. for 2 h and cooled to rt. The mixture was filtered; the filter cake was washed with three 150 mL portions of tert-butyl methyl ether to compound 8. LC-MS: m/e=253, 255 [M+H]⁺.

### Step 3:

To a stirred solution of 18.5 g (73.4 mmol) of compound 8 in 120 mL of ethanol and 80 mL of H₂O were added 15.7 g (293.6 mmol) of NH₄Cl and 20.5 g (367 mmol) of iron powder. The mixture was stirred at 80° C. for 2 h under nitrogen atmosphere and cooled to rt. The mixture was filtered; the filter cake was washed with three 150 mL portions of dichloromethane. The filtrate was extracted with three 300 mL portions of dichloromethane. The combined organic extracts were washed with brine, and dried over anhydrous Na₂SO₄. After filtration, the filtrate was concentrated under vacuum to afford compound 9. LC-MS: m/e=223, 225 [M+H]⁺.

### Step 4:

To a stirred 125 mL of ice water was added 100 mL of conc. H₂SO₄ dropwise at 0° C. Then 10.0 g (45.0 mmol) of compound 9 was added in several batches at 0° C. After 10 min, a solution of 6.2 g (90 mmol) of NaNO₂ in 10 mL of H₂O was added dropwise at 0° C. After 20 min, a solution of 20.2 g (135 mmol) of NaI in 10 mL of H₂O was added dropwise. The mixture was stirred for additional 30 min at 0° C., then warmed to 60° C. and stirred for 2 h. The mixture was diluted with 150 mL of water, adjusted to pH 8-9 with 2 N NaOH and extracted with three 200 mL of ethyl acetate. The combined organic extracts were washed with brine, and dried over anhydrous Na₂SO₄. After filtration, the filtrate was concentrated under vacuum to afford a crude, which was purified by chromatography on silica gel column eluting with 0 to 1% gradient of ethyl acetate in petroleum ether to afford compound 10. LC-MS: m/e=334, 336 [M+H]⁺.

### Step 5:

To a stirred solution of 5.2 g (15.6 mmol) of compound 10 in 80 mL of dichloromethane was added 8.05 g (46.8 mmol) of m-CPBA in several batches at 0° C. The reaction mixture was stirred at room temperature overnight and filtered; the filtrate was diluted with 100 mL of water, adjusted to pH 7-8 with saturated NaHCO₃ solution. It was extracted with three 80 mL portions of dichloromethane; the combined organic extracts were washed with brine, and dried over Na₂SO₄. After filtration, the filtrate was concentrated to afford a crude, which was purified by chromatography on silica gel column eluting with 0 to 15% gradient of ethyl acetate in petroleum ether to afford compound 11. LC-MS: m/e=350, 352 [M+H]⁺.

### Step 6:

To a stirred solution of 2.5 g (7.14 mmol) of compound 11 in 60 mL of chloroform was added 7.7 g (50.22 mmol) of POCl₃ dropwise. The mixture was stirred at 80° C. for 2 h and cooled to room temperature. The reaction was quenched by addition of 80 mL of water at 0° C. It was adjusted pH to 7-8 with saturated NaHCO₃ solution, and extracted with three 80 mL portions of dichloromethane. The combined organic extracts were washed with brine, and dried over anhydrous Na₂SO₄. After filtration, the filtrate was concentrated under vacuum to afford a crude, which was purified by chromatography on silica gel column eluting with 0 to 3% gradient of ethyl acetate in petroleum ether to afford compound 12. LC-MS: m/e=368, 370 [M+H]⁺.

### Step 7:

To a stirred solution of 0.20 g (1.63 mmol) of compound 12 in 6 mL of 1,4-dioxane was added 4 mL of ammonium hydroxide. The resulting solution in a sealed tube was stirred at 120° C. overnight and cooled to rt. The mixture was extracted with three 10 mL portions of ethyl acetate; the combined organic extracts were washed with 10 mL of brine and dried over anhydrous Na₂SO₄. After filtration, the filtrate was concentrated under vacuum to afford a crude, which was purified by chromatography on silica eluted with eluting with 0% to 20% ethyl acetate in petroleum ether to afford compound 13. LC-MS: m/e=349, 351 [M+H]⁺.

### Step 8:

To a stirred solution of 0.80 g (2.3 mmol) of compound 13 in 10 mL of DCM and 10 mL of THF were added 1.9 mL (13.7 mmol) of Et₃N and 0.42 g (3.4 mmol) of DMAP in portions at 0° C. To the mixture was added 0.65 mL (9.1 mmol) of acetyl chloride at 0° C. The mixture was stirred at RT for 48 h and then extracted with three 20 mL portions of EtOAc. The combined organic layers were washed with brine and dried over anhydrous Na₂SO₄. After filtration, the filtrate was concentrated to give a residue, which was dissolved in 15 mL of THF and added 2 mL of NH₃.H₂O. The mixture was stirred at RT for 30 min and extracted with three 30 mL portions of DCM. The combined organic layers were washed with brine and dried over anhydrous Na₂SO₄. After filtration, the filtrate was concentrated to give a residue, which was purified by silica gel column chromatography eluting with 3% MeOH in DCM to afford compound 14. LC-MS: m/e=391 [M+H]⁺.

### Step 9:

To a stirred solution of 0.70 g (1.8 mmol) of compound 14 in 20 mL of DMF were added 0.49 g (3.6 mmol) of K₂CO₃ and 0.97 g (7.2 mmol) of 3-bromo-2-methylprop-1-ene in portions.

The mixture was stirred at RT overnight and extracted with three 50 mL portions of EtOAc. The combined organic layers were washed with brine and dried over anhydrous Na₂SO₄. After filtration, the filtrate was concentrated to give a residue, which was purified by reverse flash chromatography [column, C18 silica gel; mobile phase, ACN in water(0.05% NH₄HCO₃), 0 to 80% gradient in 40 min; detector, UV 246 nm] to afford intermediate 15. LC-MS: m/e=445 [M+H]⁺.

### 4. Synthesis of Intermediate 20:

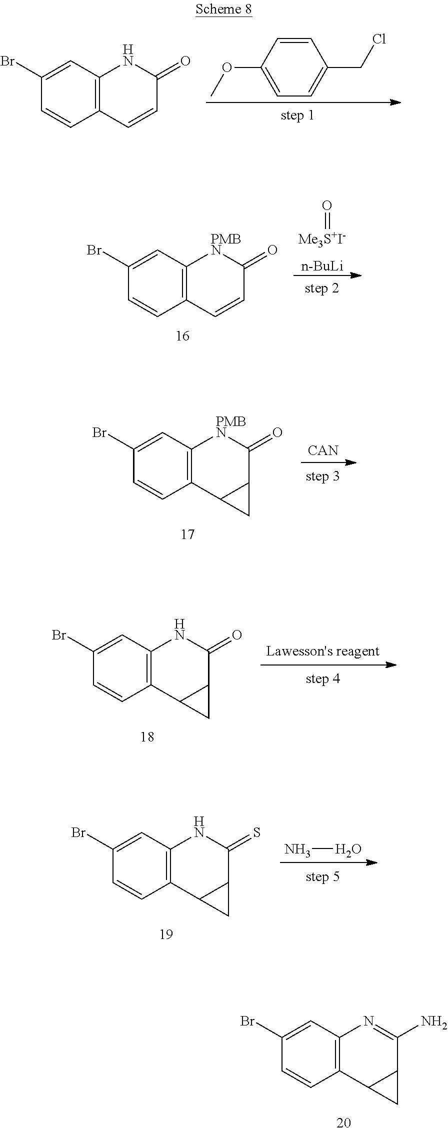

### Step 1:

To a stirred solution of 9.02 g (40.3 mmol) of 7-bromo-1H-quinolin-2-one in 360 mL of DMF were added 6.0 g (250 mmol) of NaH and 21.8 mL (161 mmol) of 4-methoxybenzyl chloride in portions at 0° C. under Ar atmosphere. The mixture was stirred at RT overnight and then quenched with H₂O. The mixture was extracted with three 150 mL portions of ethyl acetate; the combined organic layers were washed with brine and dried over anhydrous Na₂SO₄. After filtration, the filtrate was concentrated to give a residue, which was purified by silica gel column chromatography eluting with 10% ethyl acetate in petroleum ether to afford compound 16. LC-MS: m/e=344 [M+H]⁺.

### Step 2:

To a stirred solution of 1.17 g (5.3 mmol) of trimethyl(oxo)-lambda-6-sulfanylium iodide in 5 mL of THF was added 2.1 mL (2.5 M, 5.2 mmol) of n-BuLi dropwise at 0° C. under nitrogen atmosphere. After stirring for 30 min at 0° C. for 30 min, to the above mixture was added 0.61 g (1.8 mmol) of compound 16 dropwise at 0° C. The mixture was stirred at RT overnight and quenched with saturated aqueous NH₄Cl and extracted with two 50 mL portions of ethyl acetate. The combined organic layers were washed with brine and dried over anhydrous Na₂SO₄. After filtration, the filtrate was concentrated to give a residue, which was purified by silica gel column chromatograph eluting with 10% ethyl acetate in petroleum ether to afford compound 17. LC-MS: m/e=358 [M+H]⁺.

### Step 3:

To a stirred solution of 4.02 g (11.2 mmol) of compound 17 in 120 mL of ACN/H₂O (9:1) was added 21.6 g (39.3 mmol) of ceric ammonium nitrate at RT. The mixture was stirred at RT overnight under air atmosphere and quenched with water. It was basified to pH 7-9 with saturated aqueous Na₂CO₃ and extracted with two 50 mL portions of ethyl acetate. The combined organic layers were washed with brine and dried over anhydrous Na₂SO₄. After filtration, the filtrate was concentrated to give a residue, which was purified by silica gel column chromatography eluting with 10% ethyl acetate in petroleum to afford compound 18. LC-MS: m/e=238 [M+H]⁺.

### Step 4:

To a stirred solution of 0.10 g (0.42 mmol) of compound 18 and in 7 mL of 1,4-dioxane was added 0.12 g (0.29 mmol) of Lawesson's reagent 1,3,2,4-dithiadiphosphetane, 2,4-bis(4-methoxyphenyl)-,2,4-disulfide at RT under argon atmosphere. The mixture was stirred at 80° C. for 1.5 h and cooled to RT. After addition of 0.59 mL of HCl (1 M), the mixture was stirred at RT for additional 1 h. It was neutralized to pH 7-9 with saturated aqueous NaHCO₃ and extracted with two 30 mL portions of ethyl acetate. The combined organic layers were washed with brine and dried over anhydrous Na₂SO₄. After filtration, the filtrate was concentrated to give a residue, which was purified by silica gel column chromatography eluting with 30% ethyl acetate in petroleum ether to afford compound 19. LC-MS: m/e=254 [M+H]⁺.

### Step 5:

To a stirred solution of 0.74 g (2.9 mmol) of compound 19 in 5 mL of MeOH were added 5.1 mL of NH₃ (7 M) in MeOH at RT under argon atmosphere. The mixture was stirred at RT for 3 h and concentrated to give a residue, which was purified by silica gel column chromatography eluting with 7 M NH₃ in MeOH:DCM (1:10) to afford racemic intermediate 20. LC-MS: m/e=237 [M+H]⁺.

### 5. Syntheses of Intermediates 22 and 23:

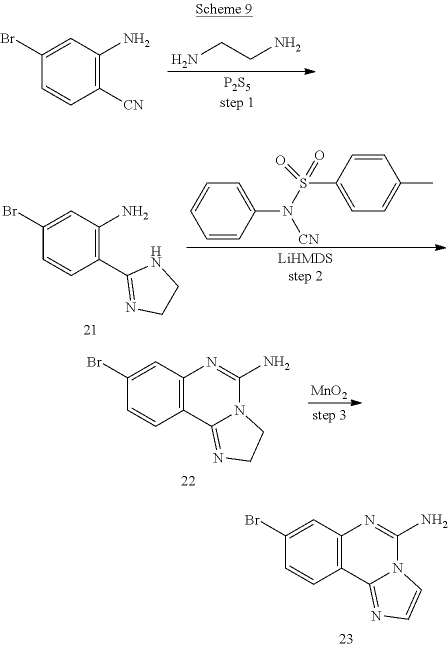

### Step 1:

To a stirred solution of 10.0 g (50.8 mmol) of 2-amino-4-bromobenzonitrile in 15.3 g (253.7 mmol) of ethylenediamine was added 0.11 g (0.51 mmol) of phosphorus pentasulfide at RT. The mixture was stirred at 90° C. overnight and cooled to RT. It was diluted with 100 mL of water; the precipitated solids were collected by filtration and washed with H₂O to afford compound 21. LC-MS: m/e=240 [M+H]⁺.

### Step 2:

To a stirred solution of 2.0 g (8.3 mmol) of compound 21 in 25 mL of 1,4-dioxane was added 2.3 g (8.3 mmol) of N-cyano-4-methyl-N-phenylbenzenesulfonamide in portions at RT under argon atmosphere. To the above mixture was added 4.7 mL (1 M) of LiHMDS dropwise over 10 min at RT. The mixture was stirred at 100° C. for 3 h and cooled to RT. The reaction was quenched by addition of 8 mL of MeOH and concentrated. The residue was purified by silica gel column chromatography eluting with 10% ethyl acetate in petroleum to afford intermediate 22. LC-MS: m/e=265 [M+H]⁺.

### Step 3:

To a stirred solution of 0.31 g (1.2 mmol) of compound 22 in 16 mL of toluene were added 1.0 g (11.8 mmol) of MnO₂ at RT under argon atmosphere. The mixture was stirred at 100° C. for 1.5 h and cooled to RT. The mixture was filtered; the filter cake was washed with two 20 mL portions of DCM. The filtrate was concentrated to give a residue, which was purified by silica gel column chromatography eluting with 10% ethyl acetate in petroleum ether to afford intermediate 23. LC-MS: m/e=263 [M+H]⁺.

### 6. Synthesis of Intermediate 27:

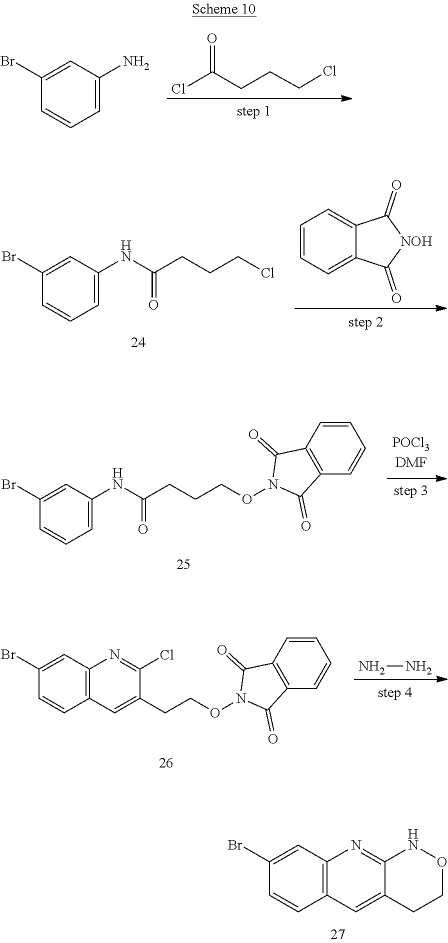

### Step 1:

To a stirred solution of 30.0 g (174 mmol) of m-bromoaniline in 440 mL of THF was added 35.3 g (349 mmol) of Et₃N at 0° C. under argon atmosphere. To the above mixture was added a solution of 29.5 g (209 mmol) of 4-chlorobutanoyl chloride in 420 mL of THF dropwise over 30 min at 0° C. The mixture was stirred for additional 1 h at RT and diluted with EA. The reaction was quenched with 50 mL of saturated aqueous ammonium chloride solution and extracted with three 500 mL portions of EA. The combined organic extracts were washed with brine and dried over anhydrous Na₂SO₄. After filtration, the filtrate was concentrated under reduced pressure. The residue was purified by silica gel column chromatography eluting with PE:DCM (1:4) to compound 24. LC-MS: m/e=276 [M+H]⁺.

### Step 2:

To a stirred solution of 25.0 g (90.4 mmol) of compound 24 and 22.1 g (136 mmol) of N-hydroxyphthalimide in 400 mL of DMSO was added 12.5 g (90.4 mmol) of K₂CO₃ in portions at RT under argon atmosphere. The mixture was stirred at 80° C. for 1 h and cooled to RT. The reaction was quenched with H₂O and extracted with three 300 mL portions of EA. The combined organic layers were washed with brine and dried over anhydrous Na₂SO₄. After filtration, the filtrate was concentrated to give a residue, which was purified by silica gel column chromatography eluting with DCM:EA (95:5) to afford compound 25. LC-MS: m/e=403[M+H]⁺.

### Step 3:

To a stirred solution of 62.7 g (409 mmol) of POCl₃ was added 8.16 g (112 mmol) of DMF dropwise at 10° C. under nitrogen atmosphere. The mixture was stirred at 10° C. for 30 min. To the above mixture was added 30.0 g (74.4 mmol) of compound 25 in portions at 10° C. The mixture was stirred at 100° C. for 1 h, cooled down to RT and concentrated under reduced pressure. The mixture was quenched by ice-water at 0° C. and neutralized to pH 7 with aqueous saturated NaHCO₃. It was extracted with three 500 mL portions of EA; the combined organic layers were washed with brine and dried over anhydrous Na₂SO₄. After filtration, the filtrate was concentrated to give a residue, which was purified by silica gel column chromatography eluting with PE:DCM (1:6) to afford compound 26. LC-MS: m/e=431 [M+H]⁺.

### Step 4:

Following the procedure described in Scheme 5, step 3, compound 26 was converted to intermediate 27. LC-MS: m/e=265[M+H]⁺.

### 7. Synthesis of Intermediate 30:

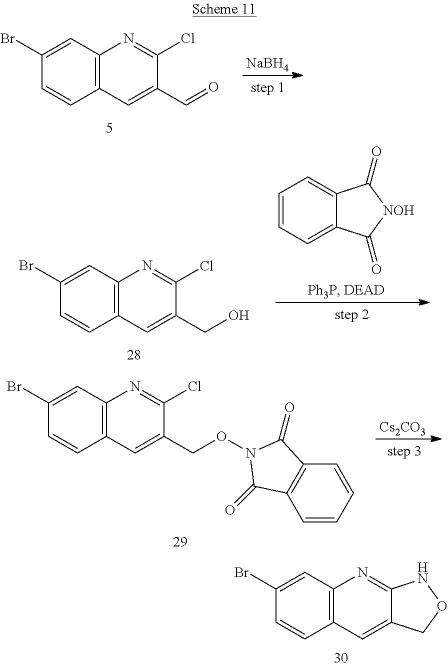

### Step 1:

To a stirred solution of 2.00 g (7.39 mmol) of compound 5 in 20 mL of ethanol was added 420 mg (11.1 mmol) of NaBH₄ in portions at RT under nitrogen atmosphere. The mixture was stirred for 1 h and concentrated under reduced pressure. It was diluted with water and extracted with three 100 mL portions of ethyl acetate. The combined organic extracts were washed with brine and dried over anhydrous Na₂SO₄. After filtration, the filtrate was concentrated under reduced pressure; the residue was purified by silica gel column chromatography eluting with PE:EA (4:1) to afford compound 28. LC-MS: m/e=272 [M+H]⁺.

### Step 2:

To a stirred solution of 1.80 g (6.61 mmol) of compound 28 in 20 mL of THF were added 1.29 g (7.91 mmol) of N-hydroxyphthalimide, 2.08 g (7.926 mmol) of PPh₃ in one portion and 1.73 g (9.91 mmol) of DEAD dropwise at 0° C. under nitrogen atmosphere. The mixture was stirred at room temperature for 1 h and diluted with water. It was extracted with three 150 mL portions of DCM; the combined organic extracts were washed with brine and dried over anhydrous Na₂SO₄. After filtration, the filtrate was concentrated under reduced pressure. The residue was purified by silica gel column chromatography eluting with DCM:PE (2:1) to afford compound 29. LC-MS: m/e=417 [M+H]⁺.

### Step 3:

To a stirred solution of 2.00 g (4.79 mmol) of compound 29 in 20 mL of dioxane was added 3.13 g (9.58 mmol) of Cs₂CO₃ at RT. The mixture was stirred at 100° C. for 24 h under nitrogen atmosphere and cooled to RT. It was concentrated under vacuum; the residue was purified by silica gel column chromatography eluting with DCM:EA (92:8) give intermediate 30. LC-MS: m/e=251 [M+H]⁺.

### 8. Synthesis of Intermediate 34:

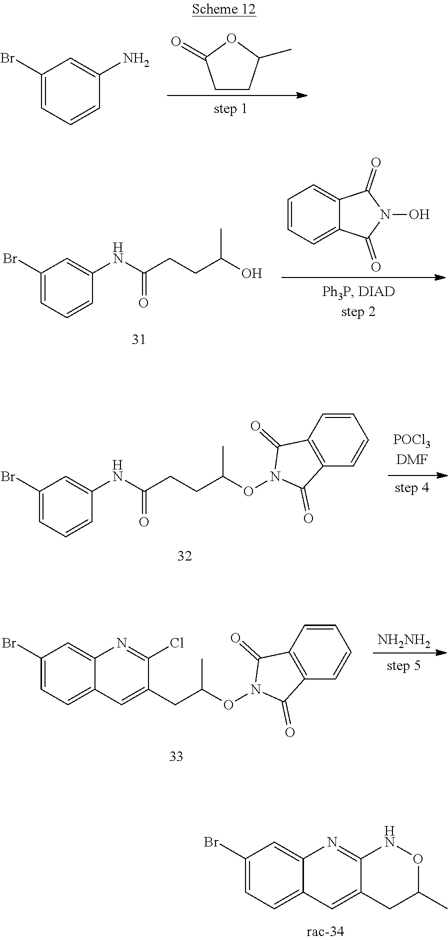

### Step 1:

To a stirred mixture of 0.93 g (23.3 mmol) of NaH in 20 mL of THF was added 2.00 g (11.6 mmol) of m-bromoaniline in 10 mL THF in portions at 0° C. under nitrogen atmosphere. After 30 min, 1.40 g (14.0 mmol) of 5-methyldihydrofuran-2(3H)-one in 10 mL THF in portions was introduced over 5 min at 0° C. The mixture was stirred at RT overnight at room temperature, quenched with ice water at 0° C., and concentrated to remove THF. The aqueous mixture was extracted with three 30 mL portions of EA; the combined organic layers were washed with brine and dried over anhydrous Na₂SO₄. After filtration, the filtrate was concentrated to give a residue, which was purified by silica gel column chromatography eluting with CH₂Cl₂/MeOH (20:1) to afford compound 31. LC-MS: m/e=272 [M+H]⁺.

### Step 2:

To a stirred solution of 2.00 g (7.35 mmol) of compound 31, 1.20 g (7.35 mmol) of N-hydroxyphthalimide and 4.82 g (18.4 mmol) of PPh₃ in 20 mL of THF was added 3.72 g (18.4 mmol) of DIAD dropwise at 0° C. under nitrogen atmosphere. After 2 h, the reaction was quenched by ice water at 0° C. and concentrated to remove THF. The resulting mixture was concentrated under reduced pressure. The aqueous mixture was extracted with three 50 mL portions of EtOAc; the combined organic layers were washed with brine and dried over anhydrous Na₂SO₄. After filtration, the filtrate was concentrated to give a residue, which was purified by reverse flash chromatography with the following conditions: column, C18 silica gel; mobile phase, 0.05% TFA in water, 55% to 65% gradient in 10 min; detector, UV 254 nm to afford compound 32. LC-MS: m/e=417 [M+H]⁺.

### Step 3:

Compound 33 was prepared from compound 32 by a similar procedure described in Scheme 5, step 2. LC-MS: m/e=445 [M+H]⁺.

### Step 4:

Following the procedure described in Scheme 5, step 3, compound 33 was converted to intermediate rac-34. LC-MS: m/e=279 [M+H]⁺.

### 9. Synthesis of Intermediate 38:

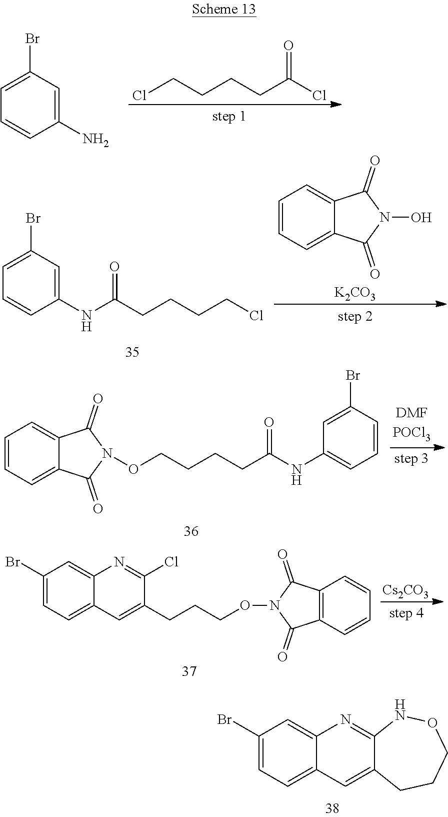

### Step 1:

Following the procedure described in Scheme 10, step 1, compound 35 was prepared from m-bromoaniline similarly. LC-MS: m/e=290 [M+H]⁺.

### Step 2:

Following the procedure described in Scheme 10, step 2, compound 36 was prepared from compound 35 similarly. LC-MS: m/e=417 [M+H]⁺.

### Step 3:

Following the procedure described in Scheme 10, step 3, compound 37 was prepared from compound 36 similarly. LC-MS: m/e=445 [M+H]⁺.

### Step 4:

Following the procedure described in Scheme 11, step 3, intermediate 38 was prepared from compound 37 similarly. LC-MS: m/e=279 [M+H]⁺.

### 10. Syntheses of Intermediates 44 and 45:

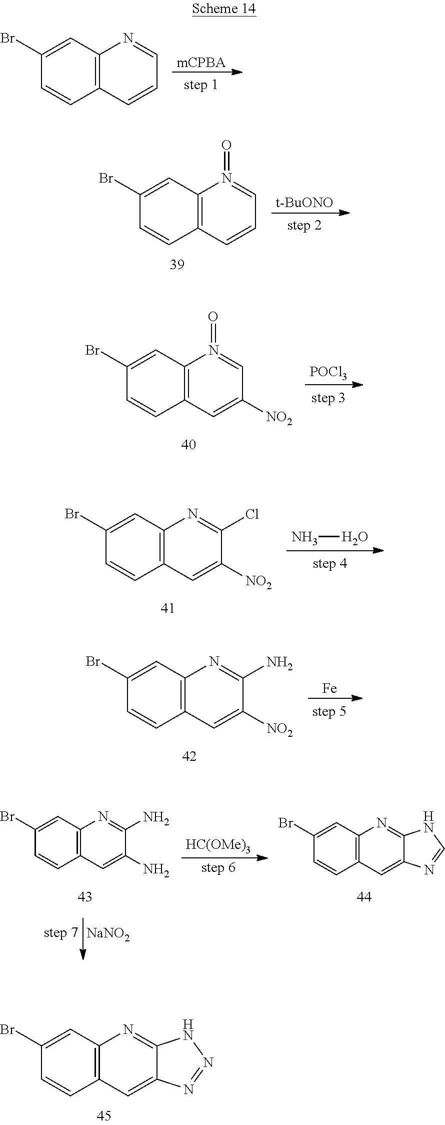

### Step 1:

Following the procedure described in Scheme 7, step 5, compound 39 was prepared from 7-bromoquinoline. LC-MS: m/e=224 [M+H]⁺.

### Step 2:

To a stirred solution of 3.00 g (13.4 mmol) of compound 39 in 50 mL of CH₃CN was added 5.52 g (53.6 mmol) of tert-butyl nitrite in portions at RT. The mixture was stirred for 24 h at 75° C., cooled to RT, and concentrated. The residue was dissolved in water and extracted with three 100 mL portions of EA. The combined organic layers were washed with brine and dried over anhydrous Na₂SO₄. After filtration, the filtrate was concentrated to give a residue, which was purified by silica gel column chromatography eluting with PE:THF (10:1) to afford compound 40. LC-MS: m/e=269 [M+H]⁺.

### Step 3:

Following the procedure described in Scheme 7, step 6, compound 40 was converted to compound 41 similarly. LC-MS: m/e=287 [M+H]⁺.

### Step 4:

Following the procedure described in Scheme 7, step 7, compound 42 was prepared from compound 41 similarly. LC-MS: m/e=268 [M+H]⁺.

### Step 5:

To a stirred solution of 850 mg (3.17 mmol) of compound 42 in 8 mL of CH₃COOH was added 885 mg (15.9 mmol) of Fe powder in portions at RT. The mixture was stirred for 1 h at 65° C., cooled to RT, and quenched by H₂O. The mixture was basified to pH 8 with saturated aqueous NaHCO₃ and extracted with three 100 mL portions of EA. The combined organic layers were washed with brine and dried over anhydrous Na₂SO₄. After filtration, the filtrate was concentrated to give a residue, which was purified by silica gel column chromatography eluting with DCM:MeOH (20:1) to afford compound 43. LC-MS: m/e=238 [M+H]⁺.

### Step 6:

A solution of 500 mg (2.10 mmol) of compound 43 in 8 mL of trimethoxymethane was stirred for 1 h at 145° C. under argon atmosphere. It was cooled to RT and concentrated. The residue was purified by silica gel column chromatography eluting with DCM:MeOH (20:1) to afford intermediate 44. LC-MS: m/e=248 [M+H]⁺.

### Step 7:

To a stirred solution of 900 mg (3.78 mmol) of compound 43 in 25 mL of acetic acid was added 50 mL (1 M in H₂O) of sodium nitrite solution at RT. The resulting mixture was stirred for 1 h at 70° C. and cooled to RT. The precipitate was collected by filtration and washed with ice water to give intermediate 45. LC-MS: m/e=249 [M+H]⁺.

### 11. Synthesis of Intermediate Rac-49:

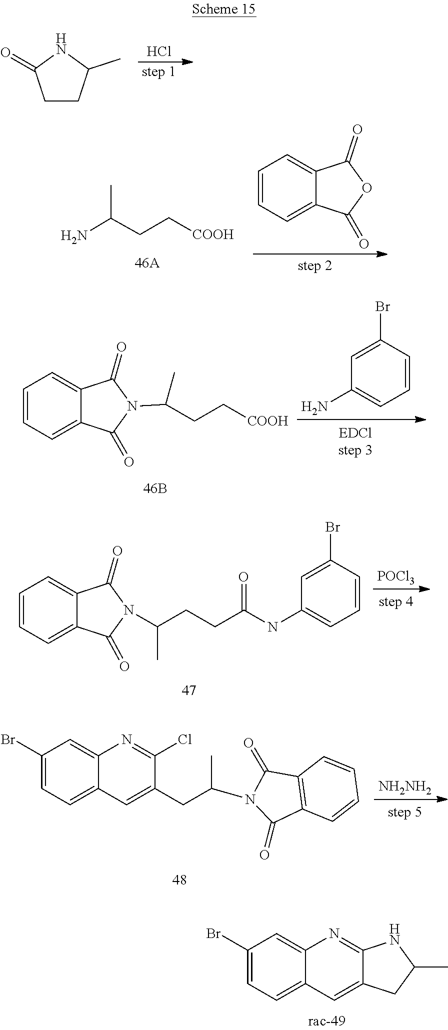

### Step 1:

Into a 250 mL round-bottom flask were added 10.0 g(101 mmol) of 5-methylpyrrolidin-2-one and 100 mL of concentrated HCl at 0° C. The mixture was stirred at 80° C. overnight under nitrogen atmosphere and concentrated. The residue was diluted with ACN to yield precipitates, which were collected by filtration and washed with ACN. The solid was dried under vacuum to afford compound 46A hydrochloride salt. LC-MS: m/e=118 [M+H]⁺.

### Step 2:

To a stirred solution of 9.30 g (79.4 mmol) of compound 46A HCl salt and 12.9 g (87.3 mmol) of phthalic anhydride in 100 mL of toluene was added 12.01 g (119 mmol) of Et₃N dropwise at RT under nitrogen atmosphere. The mixture was stirred for 3 h at 80° C. and concentrated under reduced pressure. The residue was dissolved in DCM and then concentrated to afford compound 46B. LC-MS: m/e=248 [M+H]⁺.

### Step 3:

To a stirred solution of 7.50 g (30.3 mmol) of compound 46B in 100 mL of CHCl₃ were added 2.61 g (15.2 mmol) of m-bromoaniline and 6.40 g (33.4 mmol) of EDCI at RT under nitrogen atmosphere. The resulting mixture was stirred for 2 h and concentrated. The residue was diluted with H₂O and extracted with three 50 mL portions of EtOAc. The combined organic layers were washed with brine and dried over anhydrous Na₂SO₄. The residue was purified by silica gel column chromatography eluting with CH₂Cl₂:EtOAc (36:1) to afford compound 47. LC-MS: m/e=401 [M+H]⁺.

### Step 4:

Compound 48 was prepared from compound 47 by a similar procedure described in Scheme 10, step 3. LC-MS: m/e=429 [M+H]⁺.

### Step 5:

Following the procedure described in Scheme 5, step 3, compound 48 was converted to intermediate rac-49. LC-MS: m/e=263 [M+H]⁺.

### 12. Synthesis of Intermediate 57:

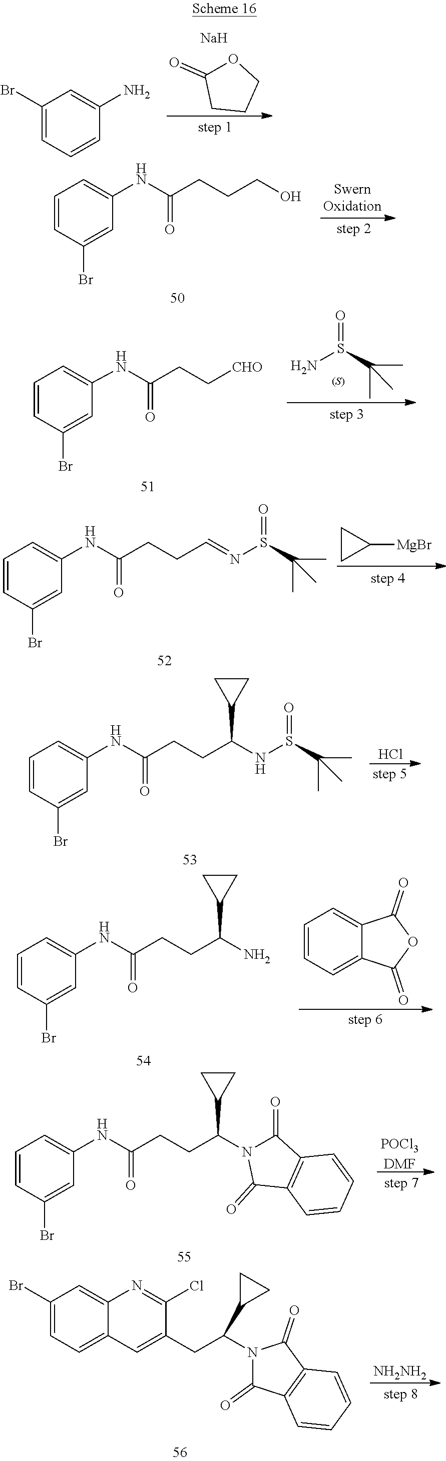

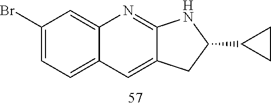 [SMILES: Brc1ccc2cc3c(nc2c1)N[C@H](C1CC1)C3]

### Step 1:

Compound 50 was prepared from m-bromoaniline similarly following the procedure described in Scheme 12, step 1. LC-MS: m/e=258 [M+H]⁺.

### Step 2:

To a stirred solution of 5.45 g (69.7 mmol) of DMSO in 180 mL of DCM was added 7.38 g (58.1 mmol) of (COCl)₂ dropwise at −78° C. under argon atmosphere. After 15 min, a solution of 6.00 g (23.2 mmol) of compound 50 in 20 mL of DCM was added in portions. The mixture was stirred for additional 50 min at −78° C.; to the above mixture was added 14.1 g (139 mmol) of Et₃N dropwise over 30 min. The reaction mixture was allowed to warm to RT and stirred overnight. It was diluted with H₂O and extracted with three 100 mL portions of DCM. The combined organic layers were washed with brine and dried over anhydrous Na₂SO₄. After filtration, the filtrate was concentrated to give a residue, which was purified by silica gel column chromatography eluting with PE:EA (4:1) to afford compound 51. LC-MS: m/e=256 [M+H]⁺.

### Step 3:

To a stirred solution of 2.00 g (7.81 mmol) of compound 51 in 5 mL of Ti(OEt)₄ were added 0.95 g (7.81 mmol) of (S)-2-methyl-2-propanesulfinamide in portions at RT under argon atmosphere. The mixture was stirred at 80° C. overnight, cooled to RT, and diluted with 30 mL of EA and 10 mL of brine. The mixture was stirred for additional 1 h. The solids were removed by filtration, and the filtrate was washed with water. The organic phase was evaporated to give a residue, which was purified by silica gel column chromatography eluting with DCM:EA (1:1) to afford compound 52. LC-MS: m/e=359 [M+H]⁺.

### Step 4:

To a stirred solution of 2.00 g (5.58 mmol) of compound 52 in 50 mL of DCM was added 11.2 mL (1 M in THF, 11.2 mmol) of cyclopropylmagnesium bromide dropwise at RT under argon atmosphere. The mixture was stirred for 1 h and quenched with saturated aqueous NH₄Cl and extracted with three 50 mL portions of EA. The combined organic layers were washed with brine and dried over anhydrous Na₂SO₄. After filtration, the filtrate was concentrated to give a residue, which was purified by silica gel column chromatography eluting with DCM:EA (4:1) to afford compound 53. LC-MS: m/e=401 [M+H]⁺.

### Step 5:

To a stirred solution of 1.35 g (3.36 mmol) of compound 53 in 5 mL of MeOH was added 5 mL of HCl (2 M in MeOH) dropwise at RT under argon atmosphere. The mixture was stirred for 1 h and concentrated to afford compound 54, which was used in the next step directly without further purification. LC-MS: m/e=297 [M+H]⁺.

### Step 6:

Compound 55 was prepared from compound 54 by a similar procedure described in Scheme 15, step 2. LC-MS: m/e=427 [M+H]⁺.

### Step 7:

Compound 56 was prepared from compound 55 by a similar procedure described in Scheme 10, step 3. LC-MS: m/e=455 [M+H]⁺.

### Step 8:

Intermediate 57 was prepared from compound 56 by a similar procedure described in Scheme 5, step 3. LC-MS: m/e=289 [M+H]⁺.

### 13. Synthesis of Intermediate 63:

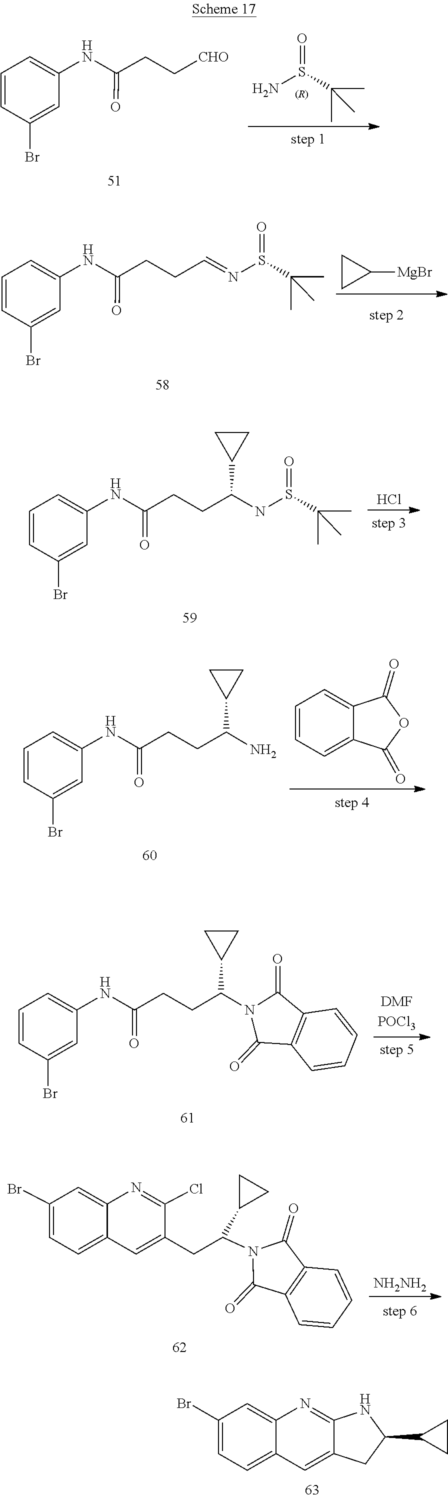

Intermediate 63 (the enantiomer of intermediate 57) was prepared from aldehyde 51 through similar reaction sequence (step 3 through 8) described in Scheme 16, using (R)-2-methyl-2-propanesulfinamide instead of (S)-2-methyl-2-propanesulfinamide. LC-MS: m/e=289 [M+H]⁺.

### 14. Synthesis of Intermediate 68:

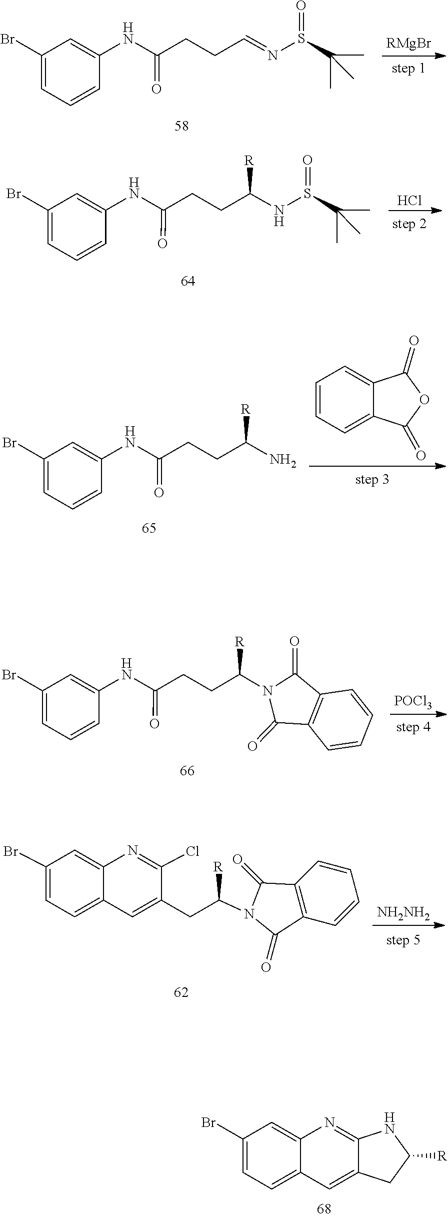

Following similar procedures described in Scheme 16, the tricyclic intermediates 68A-68F listed in Table 4 were prepared from compound 52 by using various Grignard reagents.

TABLE 4
Syntheses of tricyclic intermediates 68A-68F

| RMgBr | | LCMS |
|---|---|---|
| Employed | Intermediate Structure | [M + H]⁺ |
| MeMgBr | 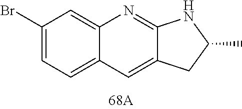 | 263 |
| EtMgBr | 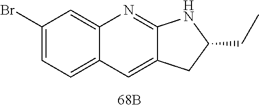 [SMILES: CC[C@@H]1Cc2cc3ccc(Br)cc3nc2N1] | 277 |
| i-PrMgBr | 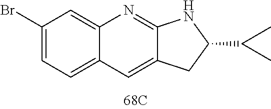 [SMILES: CC(C)[C@@H]1Cc2cc3ccc(Br)cc3nc2N1] | 291 |
| t-BuMgBr | 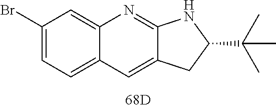 [SMILES: CC(C)(C)[C@@H]1Cc2cc3ccc(Br)cc3nc2N1] | 305 |
| AllyIMgBr | 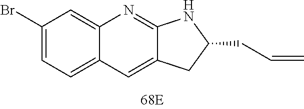 [SMILES: C=CC[C@@H]1Cc2cc3ccc(Br)cc3nc2N1] | 289 |
| PhMgBr | 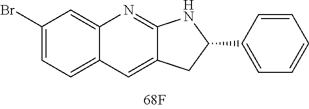 [SMILES: Brc1ccc2cc3c(nc2c1)N[C@H](c1ccccc1)C3] | 325 |

### 15. Synthesis of Intermediate 70:

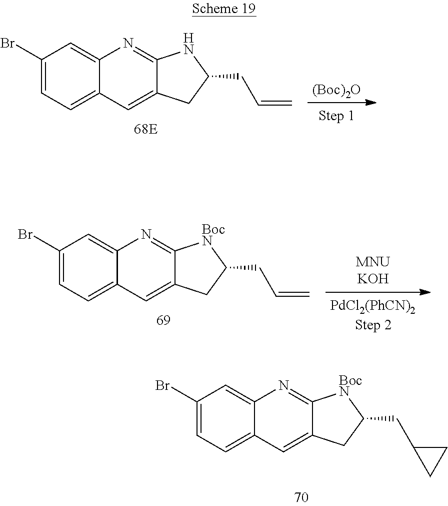 [SMILES: "(Boc)2O" ; C=CC[C@@H]1Cc2cc3ccc(Br)cc3nc2N1 ; C=CC[C@@H]1Cc2cc3ccc(Br)cc3nc2N1C(=O)OC(C)(C)C ; CC(C)(C)OC(=O)N1c2nc3cc(Br)ccc3cc2C[C@H]1CC1CC1]

### Step 1:

To a stirred solution of 280 mg (0.968 mmol) of intermediate 68E in 5 mL of DCM were added 189 mg (2.91 mmol) of TEA, 38.0 mg (0.484 mmol) of DMAP and 163 mg (1.16 mmol) of (Boc)₂O dropwise at RT under argon atmosphere. The mixture was stirred for 2 h at 30° C. and concentrated. The mixture was diluted with water and extracted with three 30 mL portions of CH₂Cl₂. The combined organic layers were washed with brine and dried over anhydrous Na₂SO₄. The residue was purified by silica gel column chromatography eluting with PE:EtOAc (5:1) to afford compound 69. LC-MS: m/e=389 [M+H]⁺.

### Step 2:

To a stirred solution of 315 mg (0.809 mmol) of compound 69 in 8 mL of DCM were added 8 mL (40% wt in H₂O) of KOH and 3.10 mg (0.008 mmol) of bis(benzonitrile)palladium chloride, and then 476.27 mg (4.620 mmol) of 1-methyl-1-nitrosourea in portions at 0° C. After 20 min, it was quenched with AcOH and concentrated. The mixture was diluted with water and extracted with three 20 mL portions of DCM. The combined organic layers were washed with brine and dried over anhydrous Na₂SO₄. The residue was purified by silica gel column chromatography eluting with PE:EtOAc (12:1) to intermediate 70. LC-MS: m/e=403 [M+H]⁺.

### 16. Synthesis of Intermediate 74:

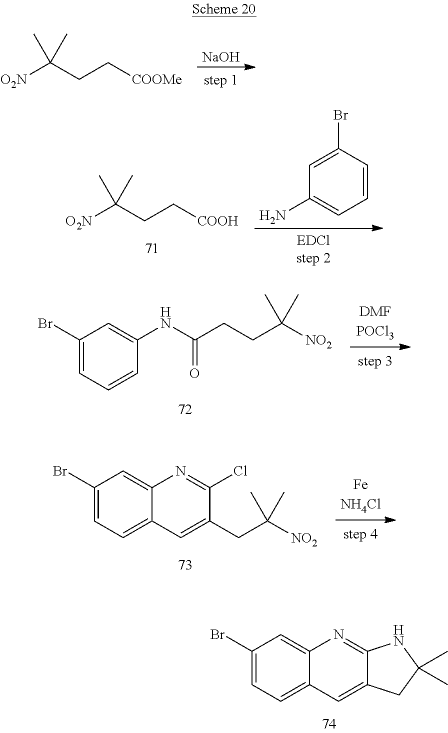

### Step 1:

To a stirred solution of 2.00 g (11.4 mmol) of methyl 4-methyl-4-nitropentanoate in 10 mL of MeOH was added 10 mL (2 M in H₂O) of NaOH dropwise at 0° C. The mixture was stirred at RT for 2 h and concentrated. The residue was diluted with H₂O, acidified to pH 6 with concentrated HCl, and concentrated. The residue was purified by silica gel column chromatography eluting with DCM:MeOH (5:1) to afford compound 71. LC-MS: m/e=181 [M+H+H₂O]⁺.

### Step 2:

Compound 72 was prepared from compound 71 by a similar procedure described in Scheme 15, step 3. LC-MS: m/e=315 [M+H]⁺.

### Step 3:

Compound 73 was prepared from compound 72 by a similar procedure described in Scheme 15, step 4. LC-MS: m/e=343 [M+H]⁺.

### Step 4:

To a stirred solution of 130 mg (0.378 mmol) of compound 73 in 6 mL of EtOH was added 106 mg (1.89 mmol) of Fe powder in portions and the solution of 81 mg (1.51 mmol) of NH₄Cl in 4 mL of H₂O at RT. The mixture was stirred at 80° C. for 4 h and concentrated. The residue was diluted with H₂O and extracted with three 10 mL portions of EtOAc. The combined organic layers were washed with brine and dried over anhydrous Na₂SO₄. The residue was purified by silica gel column chromatography eluting with PE:EtOAc (6:1) to afford intermediate 74. LC-MS: m/e=277 [M+H]⁺.

### 17. Synthesis of Intermediate 82:

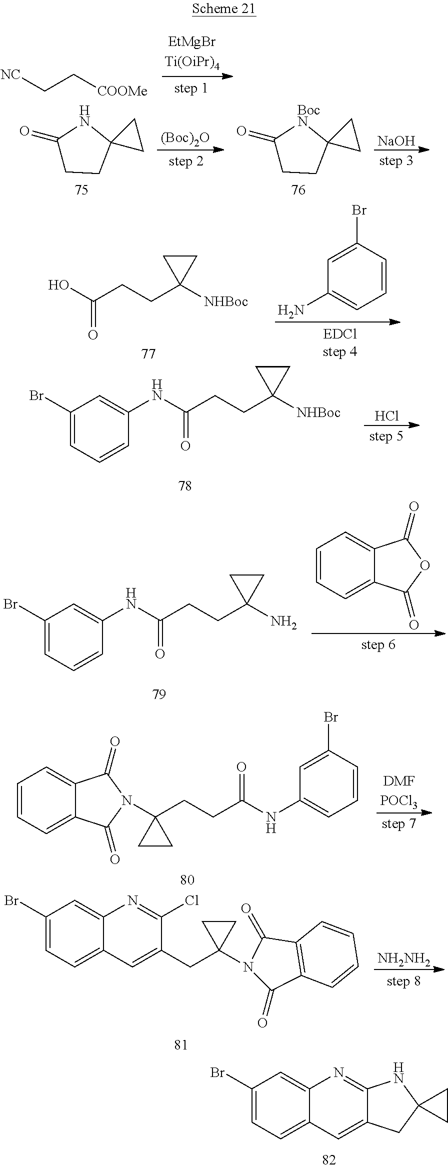

### Step 1:

To a stirred solution of 4.50 g (39.8 mmol) of methyl 3-cyanopropanoate and 12.4 g (43.8 mmol) of Ti(Oi-Pr)₄ in 160 mL of Et₂O was added 44.8 mL (2 M in Et₂O, 87.5 mmol) of EtMgBr dropwise at RT. After 2 h, it was diluted with 50 mL DCM, and concentrated. The residue was purified by silica gel column chromatography eluting with CH₂Cl₂:MeOH (50:1) to afford compound 75. LC-MS: m/e=112 [M+H]⁺.

### Step 2:

To a stirred solution of 4.30 g (38.7 mmol) of compound 75 in 50 mL of ACN were added 16.9 g (77.4 mmol) of Boc₂O and 4.73 g (38.7 mmol) of DMAP at RT. After 2 h, it was concentrated. The mixture was diluted with water and extracted with three 80 mL portions of EA. The combined organic layers were washed with brine and dried over anhydrous Na₂SO₄. After filtration, the filtrate was concentrated to give a residue, which was purified by silica gel column chromatography eluting with PE:EtOAc (10:1) to afford compound 76. LC-MS: m/e=212 [M+H]⁺.

### Step 3:

To a stirred solution of 4.70 g (22.2 mmol) of compound 76 in 40 mL of MeOH was added 20 mL (2 M in H₂O) of NaOH dropwise at 0° C. The mixture was stirred at RT for 1 h and concentrated. The residue was diluted with water, acidified to pH5 with concentrated HCl, and extracted with three 200 mL portions of EtOAc. The combined organic layers were washed with brine and dried over anhydrous Na₂SO₄. After filtration, the filtrate was concentrated under reduced pressure to afford compound 77, which was used in the next step directly without further purification. LC-MS: m/e=230 [M+H]⁺.

### Step 4:

Compound 78 was prepared from compound 77 by a similar procedure described in Scheme 15, step 3. LC-MS: m/e=383 [M+H]⁺.

### Step 5:

Into a 100 mL round-bottom flask were added 7.50 g (19.6 mmol) of compound 78 and 50 mL of HCl (4 M in 1,4-dioxane) at RT. The mixture was stirred for 1 h and concentrated to afford compound 79, which was used in the next step directly without further purification. LC-MS: m/e=283 [M+H]⁺.

### Step 6:

Following the procedure described in Scheme 15, step 2, compound 80 was prepared from compound 79. LC-MS: m/e=413 [M+H]⁺.

### Step 7:

Following the procedure described in Scheme 10, step 3, compound 81 was prepared from compound 80. LC-MS: m/e=441 [M+H]⁺.

### Step 8:

Following the procedure described in Scheme 5, step 3, intermediate 82 was prepared from compound 81. LC-MS: m/e=275 [M+H]⁺.

### 18. Syntheses of Intermediates 85 and 87:

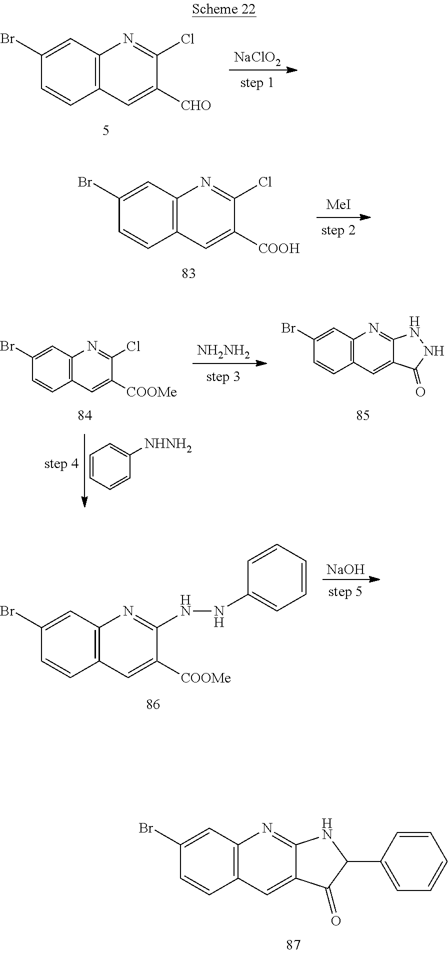

### Step 1:

To a stirred solution of 2.00 g (7.39 mmol) of compound 5 in 127 mL of n-butanol were added a solution of 6.74 g (56.2 mmol) of NaH₂PO₄ and 6.69 g (73.9 mmol) of NaClO₂ in 48 mL of H₂O dropwise at RT. After 2 h, it was concentrated. The residue was diluted with H₂O and washed with three 60 mL portions of EtOAc. The aqueous layer was acidified to pH 4 with HCl solution. The precipitated solids were collected by filtration and washed with H₂O. The solid was dissolved in MeOH and concentrated to afford compound 83. LC-MS: m/e=286 [M+H]⁺.

### Step 2:

To a stirred solution of 1.50 g (5.24 mmol) of compound 83 in 35 mL of DMF were added 2.17 g (15.7 mmol) of K₂CO₃ and 1.86 g (13.1 mmol) of CH₃I at RT. After 1 h, it was diluted with H₂O. The solid was collected by filtration and washed with H₂O to give compound 84. LC-MS: m/e=300 [M+H]⁺.

### Step 3:

To a stirred solution of 490 mg (1.63 mmol) of compound 84 in 10 mL of ethyl alcohol were added 326 mg (6.52 mmol) of hydrazine hydrate dropwise at RT. The mixture was stirred at 60° C. overnight under nitrogen atmosphere. The mixture was filtered; the filter cake was washed with three 20 mL portions of EtOH and dried to give intermediate 85. LC-MS: m/e=264 [M+H]⁺.

### Step 4:

To a stirred solution of 400 mg (1.33 mmol) of compound 84 in 5 mL of 1-pentanol was added 576 mg (5.32 mmol) of phenylhydrazine in portions at RT. The mixture was stirred for 1 h at 140° C. under argon atmosphere and concentrated. It was diluted with H₂O and extracted with three 20 mL portions of EtOAc. The combined organic layers were washed with brine and dried over anhydrous Na₂SO₄. The residue was purified by silica gel column chromatography eluting with PE:EtOAc (16:1) to afford compound 86. LC-MS: m/e=372 [M+H]⁺.

### Step 5:

To a stirred solution of 220 mg (0.591 mmol) of compound 86 in 2 mL of THF was added 6 mL (0.5 N in H₂O) of NaOH dropwise at RT under argon atmosphere. The mixture was stirred for 1 h at 80° C. and concentrated. The residue was acidified to pH 4 with concentrated HCl. The precipitated solids were collected by filtration and washed with water and acetone to afford intermediate 87. LC-MS: m/e=340 [M+H]⁺.

### 19. Synthesis of Intermediate 89:

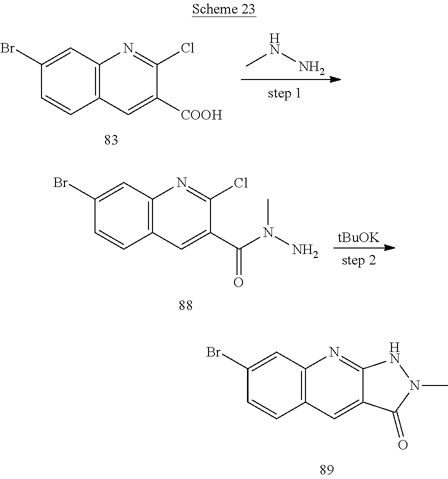

### Step 1:

To a stirred solution of 1.00 g (3.49 mmol) of compound 83 in 40 mL of DCM was added 0.45 g (3.49 mmol) of DIEA dropwise at RT under argon atmosphere. After 10 min, 0.40 g (3.49 mmol) of MsCl was added dropwise at −45° C. The mixture was warmed to RT and stirred for additional 10 min. To the above mixture were added 2.70 g (20.9 mmol) of DIEA and 0.48 g (10.471 mmol) of methyl hydrazine dropwise at −40° C. It was warmed to RT warm to room temperature and concentrated. The residue was diluted with water and extracted with three 20 mL portions of EA. The combined organic layers were washed with brine and dried over anhydrous Na₂SO₄. After filtration, the filtrate was concentrated to give a residue, which was purified by silica gel column chromatography eluting with DCM:MeOH (97:3) to afford compound 88. LC-MS: m/e=314 [M+H]⁺.

### Step 2:

To a stirred solution of 600 mg (1.91 mmol) of compound 88 in 8 mL of dimethylformamide was added 428 mg (3.82 mmol) of t-BuOK in portions at RT under argon atmosphere. The mixture was stirred for 1 h at 85° C., cooled to RT, and concentrated. The residue was dissolved in EA and stirred 16 h at RT. The precipitated solids were collected by filtration and washed with EA to afford intermediate 89, which was used in the next step directly without further purification. LC-MS: m/e=278 [M+H]⁺.

### 20. Synthesis of Intermediate 93:

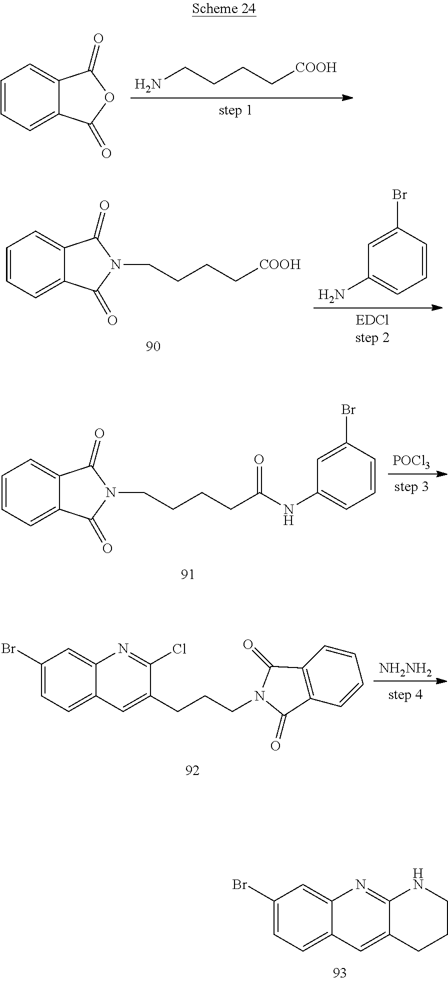

### Step 1:

Following the procedure described in Scheme 15, step 2, compound 90 was prepared from 5-aminovaleric acid and phthalic anhydride. LC-MS: m/e=248 [M+H]⁺.

### Step 2:

Following the procedure described in Scheme 15, step 3, compound 91 was prepared from compound 90. LC-MS: m/e=401 [M+H]⁺.

### Step 3:

Following the procedure described in Scheme 5, step 2, compound 92 was prepared from compound 91. LC-MS: m/e=429 [M+H]⁺.

### Step 4:

Following the procedure described in Scheme 5, step 3, intermediate 93 was prepared from compound 92. LC-MS: m/e=263 [M+H]⁺.

21. Synthesis of Intermediate rac-98:

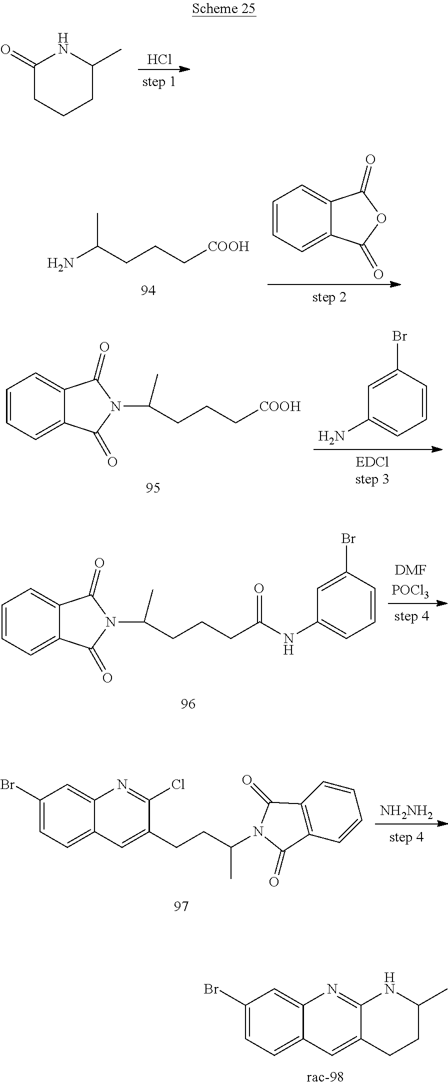

### Step 1:

Following the procedure described in Scheme 15, step 1, compound 94 was prepared from compound 6-methylpiperidin-2-one. LC-MS: m/e=132 [M+H]⁺.

### Step 2:

Following the procedure described in Scheme 15, step 2, compound 95 was prepared from compound 94. LC-MS: m/e=262 [M+H]⁺.

### Step 3:

Following the procedure described in Scheme 15, step 3, compound 96 was prepared from compound 95. LC-MS: m/e=415 [M+H]⁺.

### Step 4:

Following the procedure described in Scheme 10, step 3, compound 97 was prepared from compound 96. LC-MS: m/e=443 [M+H]⁺.

### Step 5:

Following the procedure described in Scheme 5, step 3, intermediate rac-98 was prepared from compound 97 similarly. LC-MS: m/e=277 [M+H]⁺.

### 22. Synthesis of Intermediate 106:

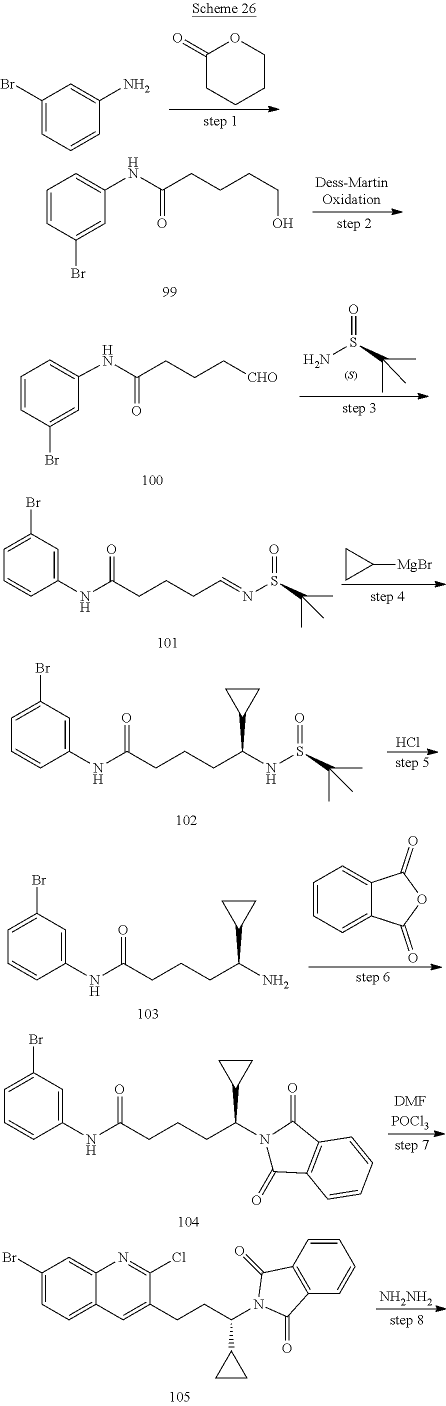

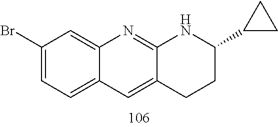 [SMILES: Brc1ccc2cc3c(nc2c1)N[C@H](C1CC1)CC3]

### Step 1:

Following the procedure described in Scheme 12, step 1, compound 99 was prepared from delta-valerolactone and m-bromoaniline. LC-MS: m/e=272 [M+H]⁺.

### Step 2:

To a stirred mixture of 70.0 g (257 mmol) of compound 99 in 2000 mL of DCM was added 164 g (386 mmol) of Dess-Martin periodinane in portions at 0° C. After 2 h, it was concentrated. The residue was dissolved in aqueous saturated Na₂CO₃ and EA. The mixture was filtered; the filter cake was washed with EtOAc. The aqueous layer was extracted with three portions 500 mL of EA; the combined organic layers were washed with brine and dried over anhydrous Na₂SO₄. After filtration, the filtrate was concentrated to afford compound 100, which was used in the next step directly without further purification. LC-MS: m/e=270 [M+H]⁺.

### Step 3:

Following the procedure described in Scheme 16, step 3, compound 101 was prepared from compound 100. LC-MS: m/e=373 [M+H]⁺.

### Step 4:

Following the procedure 4 described in Scheme 16, step, compound 102 was prepared from compound 101. LC-MS: m/e=415 [M+H]⁺.

### Step 5:

Following the procedure described in Scheme 16, step 5, compound 103 was prepared from compound 102. LC-MS: m/e=311 [M+H]⁺.

### Step 6:

Following the procedure described in Scheme 15, step 2, compound 104 was prepared from compound 103. LC-MS: m/e=441 [M+H]⁺.

### Step 7:

Following the procedure described in Scheme 10, step 3, compound 105 was prepared from compound 104. LC-MS: m/e=469 [M+H]⁺.

### Step 8:

Following the procedure described in Scheme 10, step 4, intermediate 106 was prepared from compound 105. LC-MS: m/e=303 [M+H]⁺.

### 23. Synthesis of Intermediate 111:

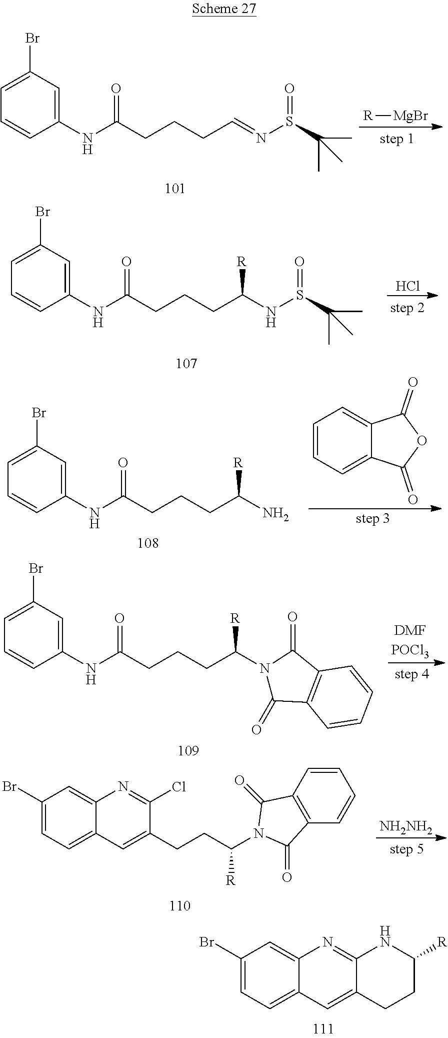

The following intermediates shown in Table 5 were prepared from intermediate 111A-111B through reaction sequences described in Scheme 26, using appropriate Grignard reagents.

TABLE 5
Syntheses of tricyclic intermediates 111A-111B

| Grignard | | LCMS |
|---|---|---|
| Reagent | Intermediate Structure | [M + H]⁺ |
| i-PrMgBr | 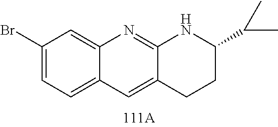 [SMILES: CC(C)[C@@H]1CCc2cc3ccc(Br)cc3nc2N1] | 305 |
| c-BuMgBr | 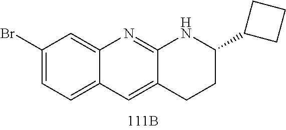 [SMILES: Brc1ccc2cc3c(nc2c1)N[C@H](C1CCC1)CC3] | 317 |

### 24. Synthesis of Intermediate 116:

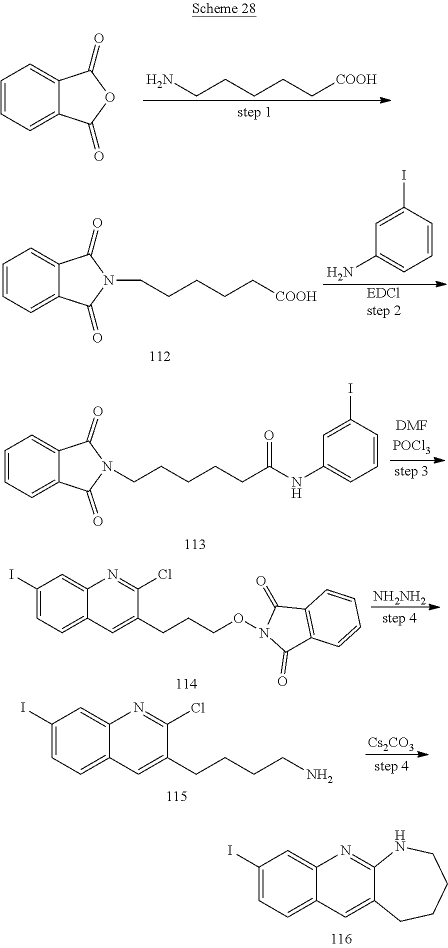

### Step 1:

Following the procedure described in Scheme 15, step 2, compound 112 was prepared from aminocaproic acid and phthalic anhydride. LC-MS: m/e=262 [M+H]⁺.

### Step 2:

Following the procedure described in Scheme 15, step 3, compound 113 was prepared from compound 112. LC-MS: m/e=463 [M+H]⁺.

### Step 3:

Following the procedure described in Scheme 10, step 3, compound 114 was prepared from compound 113. LC-MS: m/e=491 [M+H]⁺.

### Step 4:

Following the procedure described in Scheme 5, step 3, compound 115 was treated with hydrazine in butanol to give compound 114. LC-MS: m/e=361 [M+H]⁺.

### Step 5:

To a stirred solution of 500 mg(1.39 mmol) of compound 115 in 12 mL of dioxane was added 904 mg (2.77 mmol) of Cs₂CO₃ portions at RT under nitrogen atmosphere. The mixture was stirred at 100° C. overnight under nitrogen atmosphere. It was cooled to RT, diluted with H₂O, and extracted with three 20 mL portions of EtOAc. The combined organic layers were washed with brine and dried over anhydrous Na₂SO₄. The residue was purified by silica gel column chromatography eluting with DCM/PE (1:1) to afford intermediate 116. LC-MS: m/e=325 [M+H]⁺.

### 25. Synthesis of Intermediates 122 and 124:

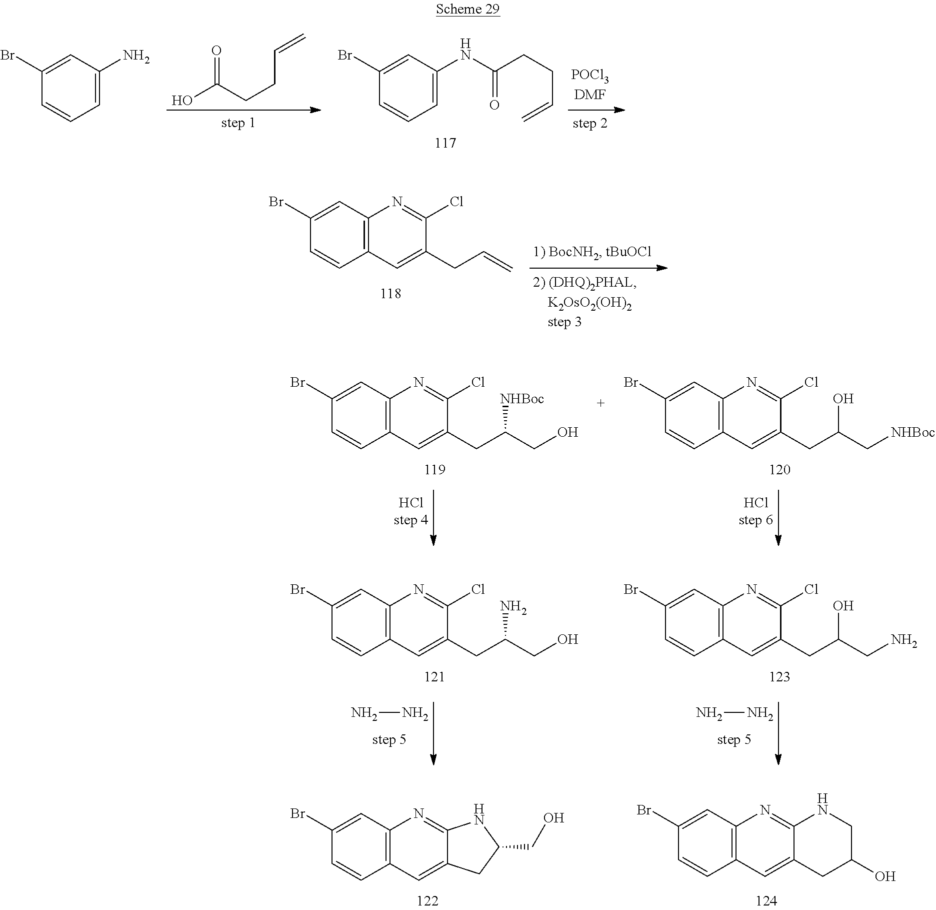

### Step 1:

Following the procedure described in Scheme 15, step 3, compound 117 was prepared from 4-pentenoic acid and 3-bromoaniline. LC-MS: m/e=254 [M+H]⁺.

### Step 2:

Following the procedure described in Scheme 6, step 2, compound 117 was converted to compound 118. LC-MS: m/e=282 [M+H]⁺.

### Step 3:

To a stirred solution of 46.4 mL of 0.4 M NaOH in H₂O were added 2.61 g (22.3 mmol) of BocNH₂ in 21.6 mL of n-PrOH and 2.07 g (19.1 mmol) of tert-butyl hypochlorite dropwise at 0° C. After 5 min, to the mixture were added 0.30 g (0.385 mmol) of (DHQ)₂PHAL in 25.2 mL of n-PrOH and 1.80 g (6.37 mmol) of compound 118 in 25.2 mL of n-PrOH and 93. mg (0.255 mmol) of K₂OsO₂(OH)₄ in 2.2 ml of 0.4 M NaOH in H₂O dropwise at 0° C. The mixture was stirred for 1 h at 0° C. under nitrogen atmosphere and concentrated. The residue was diluted with water and extracted with three 150 mL portions of EA. The combined organic layers were washed with brine, dried over anhydrous Na₂SO₄, filtered, and concentrated. The residue was purified by silica gel column chromatography eluting with PE:EtOAc (2:1) to afford compound 119 and 120. LC-MS for compound 119, m/e=415 [M+H]⁺. LC-MS for compound 120, m/e=415 [M+H]⁺.

### Step 4:

Following the procedure described in Scheme 21, step 5, compound 119 was converted to compound 121. LC-MS: m/e=315 [M+H]⁺.

### Step 5:

Following the procedure described in Scheme 5, step 3, compound 121 was converted to intermediate 122. LC-MS: m/e=279 [M+H]⁺.

### Step 6:

Following the procedure described in Scheme 21, step 5, compound 120 was converted to compound 123. LC-MS: m/e=315 [M+H]⁺.

### Step 7:

Following the procedure described in Scheme 5, step 3, compound 123 was converted to intermediate 124. LC-MS: m/e=279 [M+H]⁺.

### Assays

Protocols that may be used to determine the recited potency for the compounds of the disclosure are describe below.

### PRMT5:MEP50 Flashplate Assay:

Ten-point curves of inhibitor compounds were made using serial threefold dilutions in DMSO (the final top concentration of compound was 10 μM, 1% DMSO). Reaction mixture consisting of 50 mM Tris-HCl (pH 8.5), 0.002% Tween 20, 0.005% BSA (Bovine Serum Albumin), 1 mM TCEP, and 1% DMSO. Substrates are freshly prepared in reaction buffer. PRMT5:MEP50 was then added into substrate solution and mixed gently. Inhibitor compound was then added and incubated for 30 min at room temperature. ³H-SAM was added to initiate the reaction. Reactions were incubated for 2 h at room temperature and quenched with 0.5 mM SAM (S-adenosyl-L-Methionine) in assay buffer. An aliquot of the reaction mix was transferred to a streptavidin-coated 384-well FlashPlate (PerkinElmer). After an incubation time of 1 h, the plate was washed and then read on a TopCount (PerkinElmer) to measure the amount of tritium incorporated into the peptide substrate. IC₅₀ was calculated using conventional curve-fitting method. The testing results for selected compounds are summarized in Table 6, wherein A represents the IC₅₀ value of <1.0 nM; B represents the IC₅₀ value of 1.0-100 nM. C represents the IC₅₀ value of >100 nM.

TABLE 6
PRMT5:MEP50 Inhibitory Activity of Representative Examples

| | PRMT5 | | PRMT5 |
|---|---|---|---|
| Compound | IC₅₀ nM | Compound | IC₅₀ nM |
| JNJ-64619178* | A | 1-14 | A |
| 1-7 | A | 2-3 | A |
| 1-8 | A | 3-8 | B |
| 1-9 | A | 3-9 | B |
| 1-10 | A | 3-10 | A |
| 1-11 | A | 3-11 | B |
| 1-12 | B | 4-3 | B |
| 1-13 | A | 1-15 | B |
| 1-16 | A | 1-17 | B |
| 1-18 | A | 1-19 | B |
| 1-20 | A | 1-21 | A |
| 1-22 | A | 1-23 | A |
| 1-24 | A | 1-25 | A |
| 1-26 | A | 1-27 | A |
| 1-28 | B | 1-29 | B |
| 1-30 | A | 1-31 | A |
| 1-32 | B | 1-33 | C |
| 1-34 | C | 1-35 | A |
| 1-36 | A | 1-37 | A |
| 1-38 | A | 1-39 | B |
| 1-40 | B | 1-41 | A |
| 3-12 | B | 3-13 | A |

*JNJ-64619178 is a reference compound, which has CAS #[2086772-26-9].

Unless otherwise indicated, all numbers expressing quantities of ingredients, properties such as molecular weight, reaction conditions, and etc. used in herein are to be understood as being modified in all instances by the term “about.” Each numerical parameter should at least be construed in light of the number of reported significant digits and by applying ordinary rounding techniques. Accordingly, unless indicated to the contrary, the numerical parameters may be modified according to the desired properties sought to be achieved, and should, therefore, be considered as part of the disclosure. At the very least, the examples shown herein are for illustration only, not as an attempt to limit the scope of the disclosure.

The terms “a,” “an,” “the” and similar referents used in the context of describing embodiments of the present disclosure (especially in the context of the following claims) are to be construed to cover both the singular and the plural, unless otherwise indicated herein or clearly contradicted by context. All methods described herein may be performed in any suitable order unless otherwise indicated herein or otherwise clearly contradicted by context. The use of any and all examples, or exemplary language (e.g., “such as”) provided herein is intended merely to better illustrate embodiments of the present disclosure and does not pose a limitation on the scope of any claim. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability.

Certain embodiments are described herein, including the best mode known to the inventors for carrying out the embodiments. Of course, variations on these described embodiments will become apparent to those of ordinary skill in the art upon reading the foregoing description. The inventor expects skilled artisans to employ such variations as appropriate, and the inventors intend for the embodiments of the present disclosure to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

What is claimed is:

1. A compound represented by a formula: 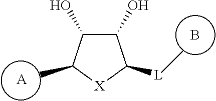
or a pharmaceutically acceptable salt thereof;
wherein 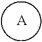
(Ring A) is an optionally substituted 4-amino-7H-pyrrolo[2,3-d]pyrimidin-7-yl; 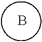
(Ring B) is an optionally substituted fused tricyclic heterocyclic ring system containing 1, 2, 3, 4, or 5 ring N atoms, 0 or 1 ring O atom, and a fused benzene ring, wherein the fused benzene ring is directly attached to L;
X is —O— or —CH₂—;
L is optionally substituted C₁₋₃ hydrocarbylene, optionally substituted —O—C₁₋₂ hydrocarbylene-, optionally substituted —S—C₁₋₂ hydrocarbylene-, or optionally substituted —NR^{A}—C₁₋₂ hydrocarbylene-; and
R^{A} is H, C₁₋₆ hydrocarbyl, C₁₋₆ heteroaryl, C₁₋₆ heterocycloalkyl, —C(O)—C₁₋₆ alkyl, —C(O)NH—C₁₋₆ alkyl, or —C(O)OC₁₋₆ alkyl.

2. The compound of claim 1, wherein Ring A comprises: 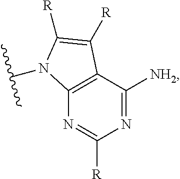
and Ring B comprises: 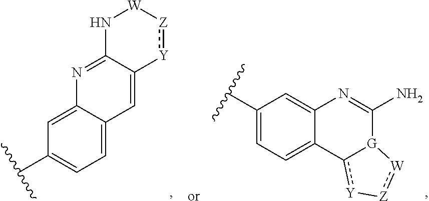
wherein each structure is optionally substituted;
G is N or CR;
Y is —N(R^{A})—, N, C(R^{C}), or —C(R^{C}R^{D})—, or —C(R^{C}R^{D})—C(R^{C}R^{D})—;
Z is a bond, —N(R^{A})—, N, C(R^{C}), or —C(R^{C}R^{D})—;
W is a bond, —N(R^{A})—, N, —O—, C(R^{C}), or —C(R^{C}R^{D})—;
dashed line represents optionally with or without a bond;
each R is independently H, F, Cl, Br, I, —NR^{A}R^{B}, C₁₋₆ hydrocarbyl, —OH, —CN, or —O—C₁₋₆ alkyl;
each R^{C}, and each R^{D} are independently H, F, Cl, Br, I, —NR^{A}R^{B}, C₁₋₆ hydrocarbyl, —OH, —CN, ═O, or —O—C₁₋₆ alkyl;
each R^{A} and each R^{B} are independently H, C₁₋₆ hydrocarbyl, C₁₋₆ heteroaryl, C₁₋₆ heterocycloalkyl, —C(O)—C₁₋₆ alkyl, —C(O)NH—C₁₋₆ alkyl, or —C(O)OC₁₋₆ alkyl; and
wherein each R, each R^{A}, each R^{B}, each R^{C}, and each R^{D} are independently optionally halogenated.

3. The compound of claim 1, wherein Ring A comprises unsubstituted 4-amino-7H-pyrrolo[2,3-d]pyrimidin-7-yl.

4. The compound of claim 1, wherein Ring B is an optionally substituted fused tricyclic heteroaromatic ring system.

5. The compound of claim 1, wherein Ring B contains one ring N atom, two ring N atoms, three ring N atoms, or four ring N atoms.

6. The compound of claim 1, wherein Ring B contains one ring O atom.

7. The compound of claim 1, wherein Ring B is optionally substituted 2,3-dihydro-1H-pyrrolo[2,3-b]quinolin-7-yl, optionally substituted 3,3-dimethyl-2,3-dihydro-1H-pyrrolo[2,3-b]quinolin-7-yl, optionally substituted 1H-pyrrolo[2,3-b]quinolin-7-yl, optionally substituted 1H-pyrazolo[3,4-b]quinolin-7-yl, optionally substituted 5-amino-2,3-dihydroimidazo[1,2-c]quinazolin-8-yl, optionally substituted 5-aminoimidazo[1,2-c]quinazolin-8-yl, optionally substituted (1aS,7bR)-2-amino-1a,7b-dihydro-1H-cyclopropa[c]quinolin-5-yl, optionally substituted (1aR,7bS)-2-amino-1a,7b-dihydro-1H-cyclopropa[c]quinolin-5-yl, optionally substituted 3,4-dihydro-1H-[1,2]oxazino[3,4-b]quinolin-8-yl, optionally substituted 1,3-dihydroisoxazolo[3,4-b]quinolin-7-yl, optionally substituted (R)-3-methyl-3,4-dihydro-1H-[1,2]oxazino[3,4-b]quinolin-8-yl, optionally substituted 3H-imidazo[4,5-b]quinolin-6-yl, optionally substituted (S)-2-methyl-2,3-dihydro-1H-pyrrolo[2,3-b]quinolin-7-yl, optionally substituted (R)-2-methyl-2,3-dihydro-1H-pyrrolo[2,3-b]quinolin-7-yl, optionally substituted (S)-2-cyclopropyl-2,3-dihydro-1H-pyrrolo[2,3-b]quinolin-7-yl, optionally substituted (R)-2-cyclopropyl-2,3-dihydro-1H-pyrrolo[2,3-b]quinolin-7-yl, optionally substituted (R)-2-ethyl-2,3-dihydro-1H-pyrrolo[2,3-b]quinolin-7-yl, optionally substituted (S)-2-isopropyl-2,3-dihydro-1H-pyrrolo[2,3-b]quinolin-7-yl, optionally substituted (S)-2-(tert-butyl)-2,3-dihydro-1H-pyrrolo[2,3-b]quinolin-7-yl, optionally substituted (R)-2-allyl-2,3-dihydro-1H-pyrrolo[2,3-b]quinolin-7-yl, optionally substituted 2,2-dimethyl-2,3-dihydro-1H-pyrrolo[2,3-b]quinolin-7-yl, optionally substituted 1′,3′-dihydrospiro[cyclopropane-1,2′-pyrrolo[2,3-b]quinolin]-7′-yl, optionally substituted 1,2,3,4-tetrahydrobenzo[b][1,8]naphthyridin-8-yl, optionally substituted (S)-2-methyl-1,2,3,4-tetrahydrobenzo[b][1,8]naphthyridin-8-yl, optionally substituted (R)-2-methyl-1,2,3,4-tetrahydrobenzo[b][1,8]naphthyridin-8-yl, optionally substituted (S)-2-cyclopropyl-1,2,3,4-tetrahydrobenzo[b][1,8]naphthyridin-8-yl, optionally substituted 2,3,4,5-tetrahydro-1H-azepino[2,3-b]quinolin-9-yl, optionally substituted (S)-(2,3-dihydro-1H-pyrrolo[2,3-b]quinolin-2-yl)methanol, or optionally substituted 1,2,3,4-tetrahydrobenzo[b][1,8]naphthyridin-3-ol.

8. The compound of claim 1, wherein X is —CH₂—.

9. The compound of claim 1, wherein X is —O—.

10. The compound of claim 1, wherein L is —CH₂—CH₂—.

11. The compound of claim 1, wherein L is —CH₂—CH₂—CH₂—

12. The compound of claim 1, wherein L is —CH₂O—.

13. The compound of claim 1, wherein L is —O—CH₂—.

14. A compound, or a pharmaceutically acceptable salt thereof, wherein the compound is optionally substituted (1S,2R,3S,5R)-3-(2-(2,3-dihydro-1H-pyrrolo[2,3-b]quinolin-7-yl)ethyl)-5-(7H-pyrrolo[2,3-d]pyrimidin-7-yl)cyclopentane-1,2-diol, optionally substituted (1S,2R,3S,5R)-3-(2-(2,3-dihydroimidazo[1,2-c]quinazolin-8-yl)ethyl)-5-(7H-pyrrolo[2,3-d]pyrimidin-7-yl)cyclopentane-1,2-diol, optionally substituted (1S,2R,3S,5R)-3-(2-(imidazo[1,2-c]quinazolin-8-yl)ethyl)-5-(7H-pyrrolo[2,3-d]pyrimidin-7-yl)cyclopentane-1,2-diol, optionally substituted (1S,2R,3S,5R)-3-(2-((1aS,7bR)-1a,7b-dihydro-1H-cyclopropa[c]quinolin-5-yl)ethyl)-5-(7H-pyrrolo[2,3-d]pyrimidin-7-yl)cyclopentane-1,2-diol, optionally substituted (1S,2R,3S,5R)-3-(2-((1aR,7bS)-1a,7b-dihydro-1H-cyclopropa[c]quinolin-5-yl)ethyl)-5-(7H-pyrrolo[2,3-d]pyrimidin-7-yl)cyclopentane-1,2-diol, optionally substituted (1S,2R,3S,5R)-3-(2-(1H-pyrazolo[3,4-b]quinolin-7-yl)ethyl)-5-(7H-pyrrolo[2,3-d]pyrimidin-7-yl)cyclopentane-1,2-diol, optionally substituted (2R,3S,4R,5R)-2-(2-(2,3-dihydro-1H-pyrrolo[2,3-b]quinolin-7-yl)ethyl)-5-(7H-pyrrolo[2,3-d]pyrimidin-7-yl)tetrahydrofuran-3,4-diol, optionally substituted (2R,3S,4R,5R)-2-(2-(2,3-dihydroimidazo[1,2-c]quinazolin-8-yl)ethyl)-5-(7H-pyrrolo[2,3-d]pyrimidin-7-yl)tetrahydrofuran-3,4-diol, optionally substituted (2R,3S,4R,5R)-2-(2-(imidazo[1,2-c]quinazolin-8-yl)ethyl)-5-(7H-pyrrolo[2,3-d]pyrimidin-7-yl)tetrahydrofuran-3,4-diol, optionally substituted (1S,2R,3S,5R)-3-(2-(1H-pyrrolo[2,3-b]quinolin-7-yl)ethyl)-5-(7H-pyrrolo[2,3-d]pyrimidin-7-yl)cyclopentane-1,2-diol, (1S,2R,3S,5R)-3-(2-(3,4-dihydro-1H-[1,2]oxazino[3,4-b]quinolin-8-yl)ethyl)-5-(7H-pyrrolo[2,3-d]pyrimidin-7-yl)cyclopentane-1,2-diol, (1S,2R,3S,5R)-3-(2-(1,3-dihydroisoxazolo[3,4-b]quinolin-7-yl)ethyl)-5-(7H-pyrrolo[2,3-d]pyrimidin-7-yl)cyclopentane-1,2-diol, optionally substituted (1R,2S,3R,5S)-3-(7H-pyrrolo[2,3-d]pyrimidin-7-yl)-5-(2-(1,3,4,5-tetrahydro-[1,2]oxazepino[3,4-b]quinolin-9-yl)ethyl)cyclopentane-1,2-diol, optionally substituted (1S,2R,3S,5R)-3-(2-(3H-imidazo[4,5-b]quinolin-6-yl)ethyl)-5-(7H-pyrrolo[2,3-d]pyrimidin-7-yl)cyclopentane-1,2-diol, optionally substituted (1S,2R,3S,5R)-3-(2-(3H-[1,2,3]triazolo[4,5-b]quinolin-6-yl)ethyl)-5-(7H-pyrrolo[2,3-d]pyrimidin-7-yl)cyclopentane-1,2-diol, optionally substituted (1S,2R,3S,5R)-3-(2-(2,3-dihydro-1H-pyrazolo[3,4-b]quinolin-7-yl)ethyl)-5-(7H-pyrrolo[2,3-d]pyrimidin-7-yl)cyclopentane-1,2-diol, optionally substituted (1R,2S,3R,5S)-3-(7H-pyrrolo[2,3-d]pyrimidin-7-yl)-5-(2-(1,2,3,4-tetrahydrobenzo[b][1,8]naphthyridin-8-yl)ethyl)cyclopentane-1,2-diol, optionally substituted (1R,2S,3R,5S)-3-(7H-pyrrolo[2,3-d]pyrimidin-7-yl)-5-(2-(2,3,4,5-tetrahydro-1H-azepino[2,3-b]quinolin-9-yl)ethyl)cyclopentane-1,2-diol, optionally substituted (2R,3S,4R,5R)-2-(2-(1H-pyrazolo[3,4-b]quinolin-7-yl)ethyl)-5-(7H-pyrrolo[2,3-d]pyrimidin-7-yl)tetrahydrofuran-3,4-diol, optionally substituted (2R,3R,4S,5R)-2-(7H-pyrrolo[2,3-d]pyrimidin-7-yl)-5-(2-(1,2,3,4-tetrahydrobenzo[b][1,8]naphthyridin-8-yl)ethyl)tetrahydrofuran-3,4-diol, or optionally substituted (1R,2S,3R,5S)-3-(7H-pyrrolo[2,3-d]pyrimidin-7-yl)-5-(2-(1,2,3,4-tetrahydropyridazino[3,4-b]quinolin-8-yl)ethyl)cyclopentane-1,2-diol.

15. The compound of claim 1, wherein the compound is deuterated.

16. The compound of claim 1, wherein each substituent, if present, of Ring A, Ring B, and L, has a molecular weight of 15 g/mol to 200 g/mol.

17. A compound, or a pharmaceutically acceptable salt thereof, wherein the compound is:

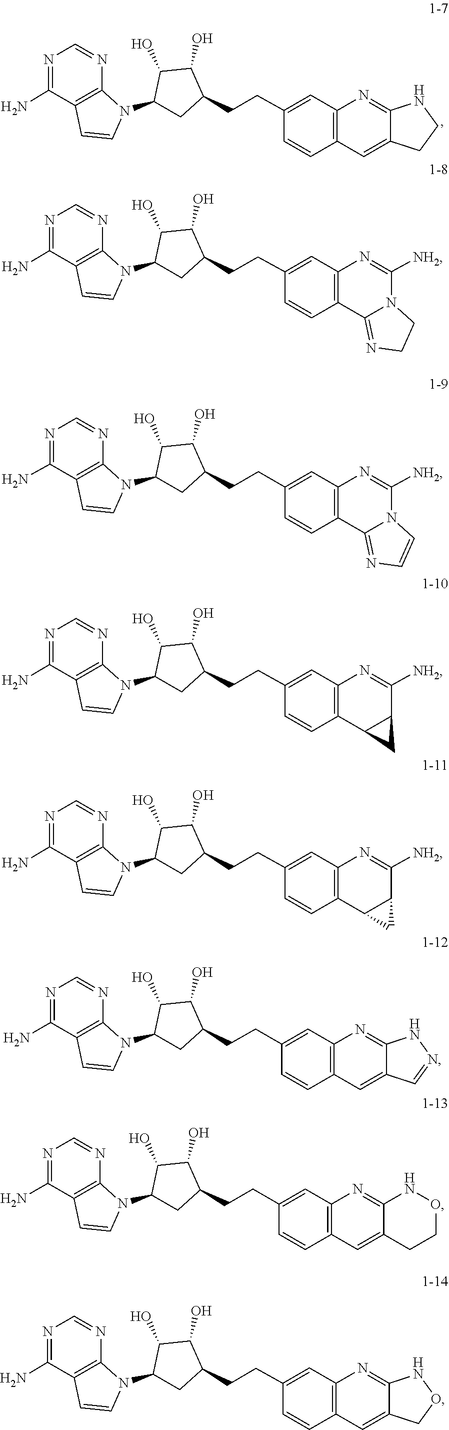

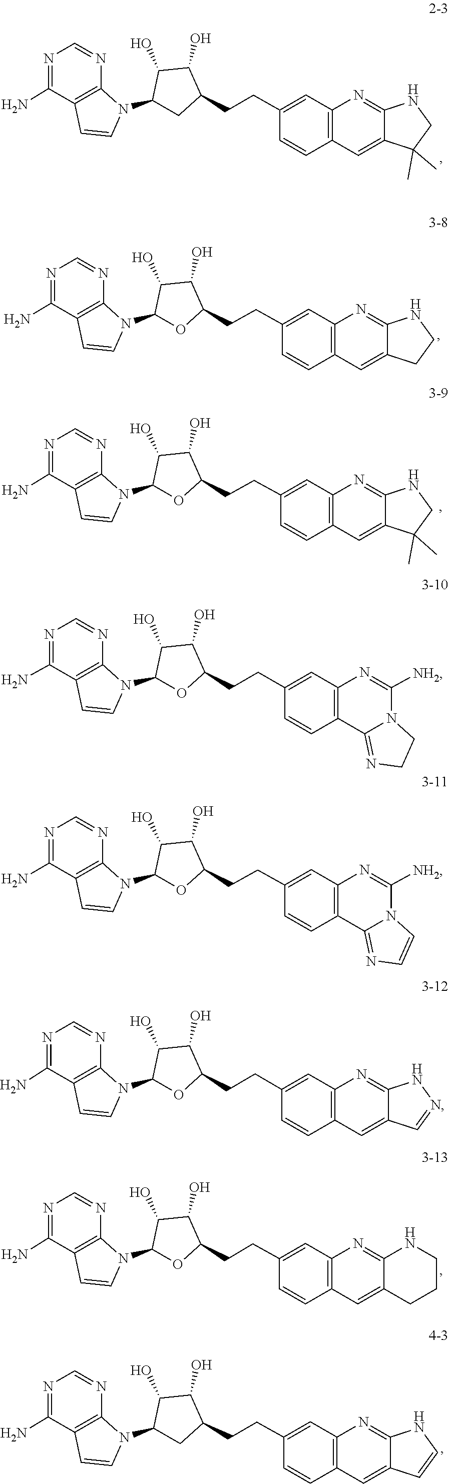

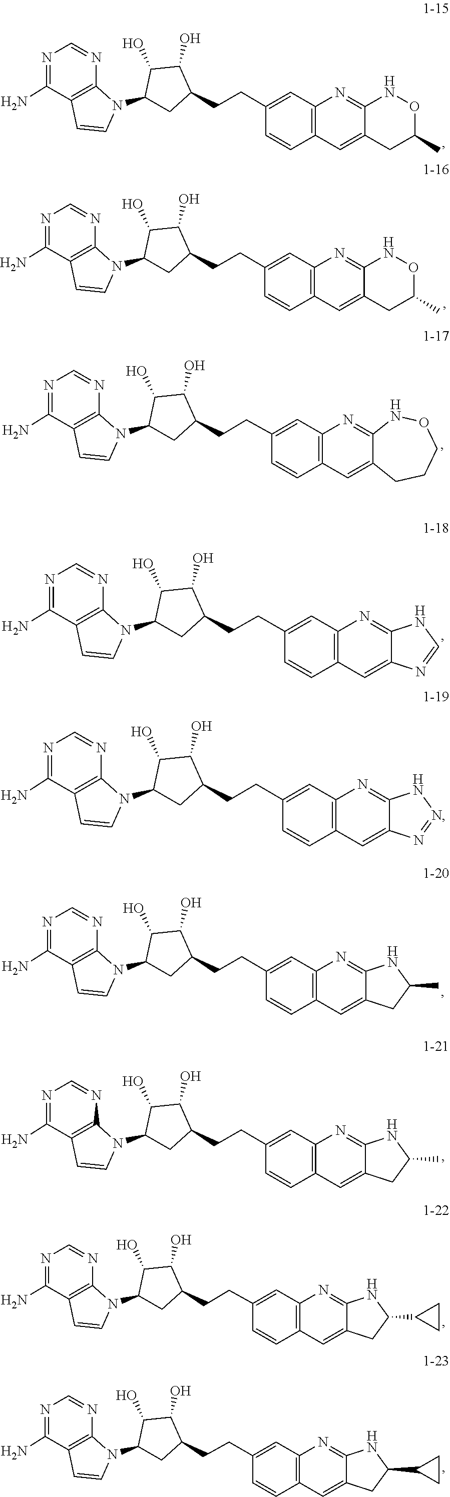

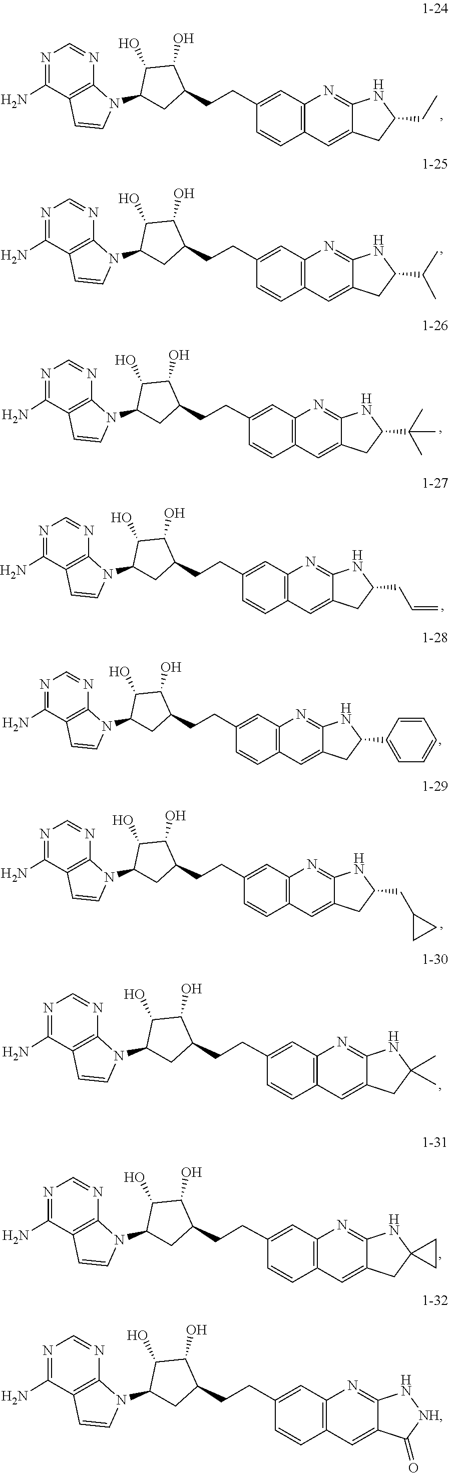

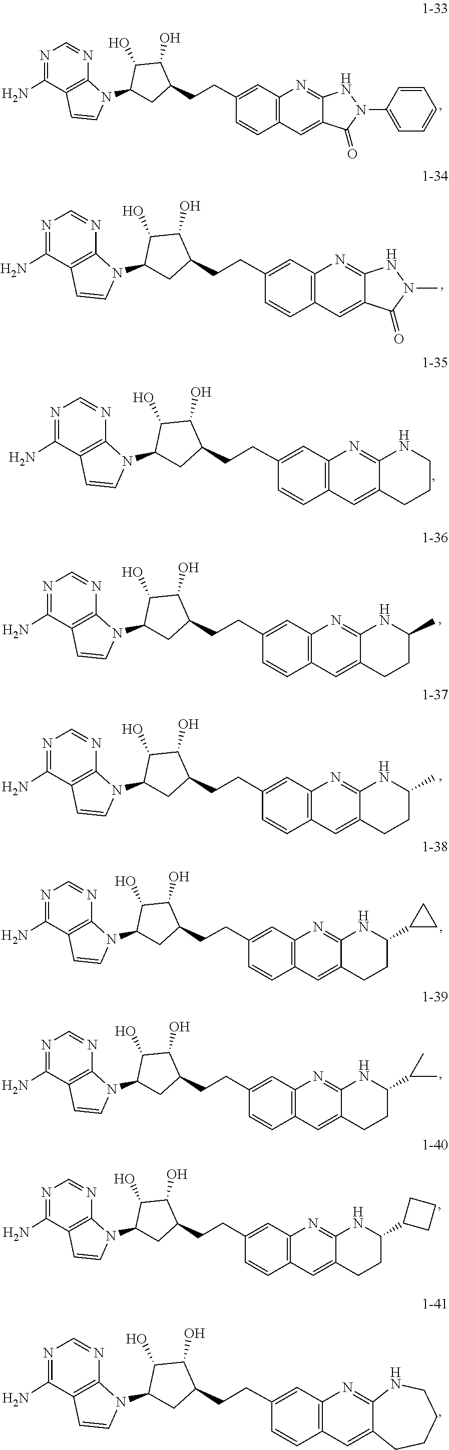

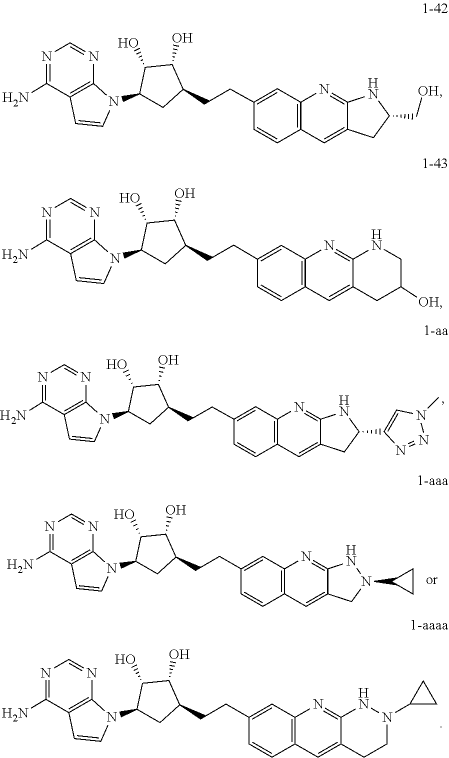

18. A method of treating cancer, infectious disease, and other PRMT5-related disease or disorder comprising administering a compound of claim 1, or a pharmaceutically acceptable salt thereof, to a patient in need thereof.

19. A pharmaceutical composition comprising a therapeutically effective amount of a compound of claim 1, or a pharmaceutically acceptable salt thereof, in combination with at least one pharmaceutically acceptable carrier.
